(19) United States
(12) Patent Application Publication
(10) Pub. No.: US 2008/0108122 A1
(43) Pub. Date: May 8, 2008

(54) Microchemical nanofactories

(75) Inventors: Brian Kevin Paul, Corvallis, OR (US); Chih-hung Chang, Corvallis, OR (US); Vincent Thomas Remcho, Corvallis, OR (US)

Correspondence Address: KLARQUIST SPARKMAN, LLP, 121 SW SALMON STREET, SUITE 1600, PORTLAND, OR, 97204, US

(73) Assignees: State of Oregon Acting by and through the State Board of Higher Education on behalf of Oregon; State University

(21) Appl. No.: 11/897,998

(22) Filed: Aug. 31, 2007

Related U.S. Application Data
(60) Provisional application No. 60/841,778, filed on Sep. 1, 2006.

(51) Int. Cl. C08G 63/00; B01D 57/02; B01J 19/00; C07C 311/01; C12N 9/00; C07D 215/00; C07D 499/04; C07J 1/00

(52) U.S. Cl. 435/183; 204/601; 204/643; 422/129; 422/198; 528/271; 540/304; 546/152; 552/201; 552/623; 564/80

(57) ABSTRACT

Embodiments of an apparatus, system, and method for chemical synthesis and/or analysis are disclosed. One embodiment of a disclosed apparatus comprises a laminated, microfluidic structure defining a reactor and a separator. Such apparatuses, or portions thereof, generally have dimensions ranging from about 1 micrometer to about 100 micrometers. To implement synthetic processes, disclosed embodiments of the apparatus generally include at least one unit operation, such as a mixer, a valve, a separator, a detector, and combinations thereof. Individual apparatuses may be coupled both in series and in parallel to form a system for making chemical compounds. An individual apparatus or a system also can be used in combination with known devices and processes.

### CROSS-REFERENCE TO RELATED APPLICATIONS

This application claims the benefit of U.S. Provisional Application No. 60/841,778 filed on Sep. 1, 2006. The entire disclosure of the provisional application is considered to be part of the present disclosure and is hereby incorporated herein by reference.

### FIELD

The present disclosure concerns embodiments of microchemical nanofactories, methods for their use, and compounds and compositions made using disclosed embodiments of the microchemical nanofactories.

### BACKGROUND

The production of specialty chemicals, such as polymeric and pharmaceutical materials, is an important aspect of the global economy. However, current commercial production methods for producing specialty chemicals are time consuming and often inefficient. Dendrimers provide one example of a class of specialty chemical that can be produced by typical synthetic methodology. Dendrimers are nanoscale macromolecules that have highly branched and core-shell structures with hollow internal voids and a number of peripheral functionalities. The chemistry of the core and the terminal functionalities can be tailored according to the specific application. As described in copending application Ser. No. 11/086,074, dendrimers have tremendous potential, but the conventional synthetic approach is time-consuming. Dendrimers can be synthesized with great precision. Ideally a certain generation of dendrimer has a single size and molecular weight rather than a broad molecular weight distribution like linear polymers.

Two general approaches to dendrimer synthesis exist. The divergent approach, arising from the seminal work of Tomalia and Newkome, initiates growth at the core of the dendrimer and continues outward by the repetition of coupling and activation steps. Convergent synthesis, first reported by Hawker and Fréchet in 1989 [J. M. J. Fréchet, Y. Jiang, C. J. Hawker, A. E. Philippides, Proc. IUPAC Int. Symp., Macromol. (Seoul), 19-20, 1989], initiates growth from the exterior of the molecule, and progresses inward by coupling end groups to each branch of the monomer. More recently, creative synthetic strategies that combined both divergent and convergent synthesis have also been developed by dendrimer chemists. A. Archut, S. Gestermann, R. Hesse, C. Kaufmann, F. Vögtle, Synlett, 546-548, 1998.

In half generations of PAMAM the terminal functionality is an ester; for full generations the terminal functionality is an amine. The structure of a generation-2 EDA-cored PAMAM is shown below.

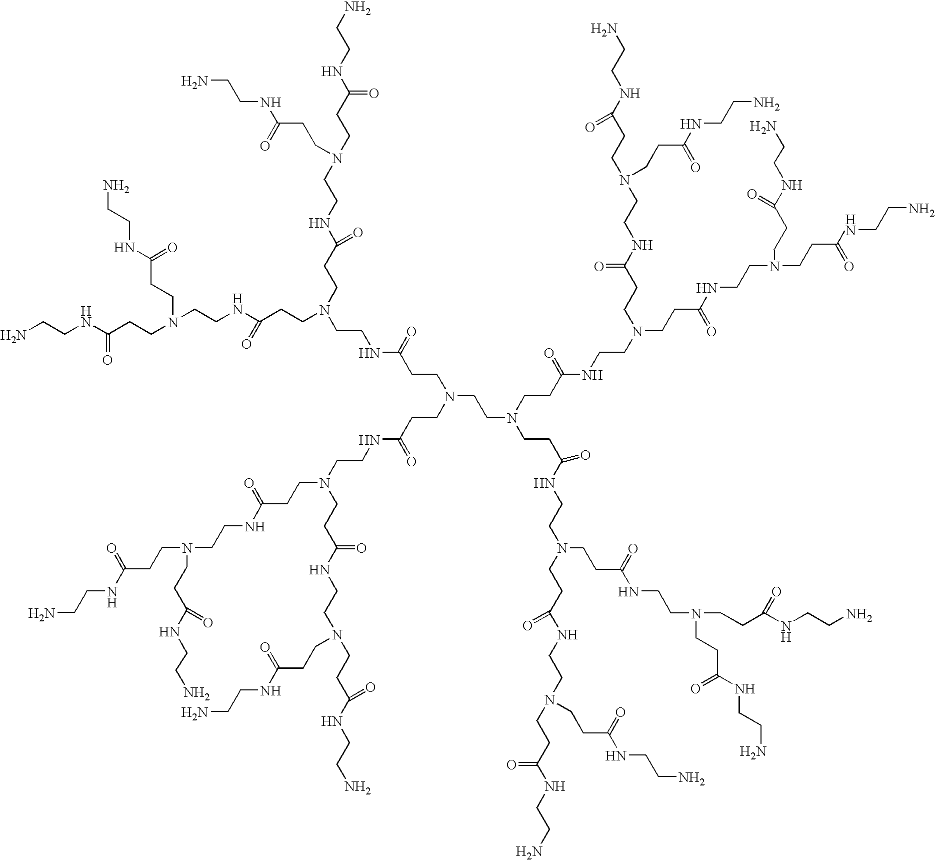

In view of the importance of specialty chemicals, such as dendrimers, new methodologies for their synthesis are desirable. Copending application Ser. No. 11/086,074, which is incorporated herein by reference, discloses embodiments of nanofactories and processes using such nanofactories that are useful for producing dendrimers, as well as other specialty chemicals. The present disclosures supplements methodologies and devices initially disclosed in the '074 application, as well as disclosing entirely new embodiments of nanofactories and methods for their use.

### SUMMARY

The present invention concerns embodiments of an apparatus and system, and method for their use, for chemical synthesis and/or analysis. One disclosed embodiment of an apparatus comprises a laminated, microfluidic structure having a reactor and typically one other unit operation device, such as a separator. Such apparatuses, or portions thereof, generally have dimensions ranging from about 1 micrometer to about 100 micrometers. “Laminated” indicates that the device is made by microlamination technology, which consists of patterning individual lamina and bonding them together to generate a monolithic device with embedded features. Individual lamina may be made from any suitable material, such as metals, intermetallics, alloys, polymeric materials (including without limitation, polydialkylsiloxanes, polycarbonates, polysulfones and polyimides), ceramics, and combinations thereof.

The reactor portion of the device typically includes a mixing section and a section useful for controlling the temperature of reactants, fluids comprising reactants, etc. For example, disclosed embodiments have a mixing section comprising an interdigital mixer (typically having plural mixing channels with a width of from about 50 μm or less and a length of about 250 μm or less.) or nozzle mixer (typically, but not necessarily, having a nozzle opening of from about 1 μm to about 10 μm and an aspect ratio of 30:1 or greater). The temperature control section may comprise either a heater, such as a thin-film heater, or a cooler.

To implement synthetic processes, disclosed embodiments of the apparatus generally include at least one valve, and often plural, selectively actuatable valves. Fluidly actuatable valves have been made in working embodiments using a fluidly deflectable, elastomeric layer.

Chemical synthesis generally requires separating unused reactants and/or byproducts from desired products. Therefore, certain embodiments of the disclosed apparatus include a separator for separating undesired materials from desired products. Examples of suitable separators, without limitation, include dielectrophoretic separators, electrophoretic separators, templated, sorbent-based separators (e.g., dendrimer-templated separators), non-templated separators, such as packed beds, capillary electrochromatographic separators, capillary zone electrophoretic separators, and combinations thereof. Detectors, including optical detectors, also can be used to detect product and other materials as they flow by, or are otherwise presented to, the detector.

A particular embodiment of the apparatus, referred to as a microchemical nanofactory, comprises at least a first inlet and a second inlet for feeding a first reagent and a second regent to a mixer. The mixer thoroughly mixes the reagents, often provided in a fluid stream, to form a mixture. The microchemical nanofactory optionally may include a heating or cooling zone for receiving the mixture. Reaction product or products, and other materials that may form or are included as solvents, reactants, etc., are received in a first microchannel that is fluidly coupled to a first separator. The device also may include a second separator, as would typically be the case for plug flow to sorbent-based separators. Moreover, selectively actuatable valves, often fluidly actuatable valves comprising a fluidly deflectable elastomeric layer, are used to guide product and other materials to the first separator, and to the second separator if present, for separating product from the other materials to form a separated product and separated materials. Additional, selectively actuatable valves may be operatively coupled to plural microchannels for guiding the product to a product microchannel and the separated materials to a separated materials microchannel.

A person of ordinary skill in the art will appreciate that individual apparatuses as described herein may be coupled to form a system for making chemical compounds. For example, such a system may comprise a first laminated, microfluidic structure defining a reactor and a separator coupled in series to at least a second laminated, microfluidic structure defining a reactor and a separator. Thus, compounds that require multi-step processes for their synthesis, such as dendrimers, can be made by performing a first reaction in a first apparatus, feeding the product to a second apparatus, typically on the same chip, to perform a second synthetic operation, and repeating such unit operations until the desired compound is completely synthesized. Moreover, individual apparatuses, or systems, can be used in parallel to make as much product as may be required.

Disclosed embodiments of the apparatus, microchemical nanofactories and/or systems can be used in a method for making a myriad of compounds using reagents now known or hereafter developed. Moreover, the apparatus is particularly useful for making compounds that use iterative reaction schemes, and further can be used to make compounds having morphological structures that resemble the morphology of the apparatus itself. For example, the apparatus may employ a fractal geometry that is useful for making, inter alia, dendrimers.

The method typically comprises providing a laminated, microfluidic apparatus defining a reactor and a separator. Reagents appropriate for making a desired chemical compound are then provided to the apparatus, and the apparatus operated to make the desired compound. For example, if the desired chemical compound is a dendrimer, the reagents may comprise ethylene diamine and methylacrylate acid.

However, a person of ordinary skill in the art will readily appreciate that the disclosed embodiments of the apparatus, microchemical nanofactory and systems are not solely useful for making dendrimers, and instead can be used to make a virtually limitless number of compounds. Solely by way of example, classes of such compounds include oligomers, biological macromolecules, simple and complex natural products, supermolecules, commercial polymeric materials, respiratory stimulants, analgesics, behavior-modifying agents, anesthetic agents, anticonvulsants, muscle relaxants, antiarrhythmic drugs, ACE inhibitors, calcium channel blocking agents, vasodilating agents, alpha-adrenergic blocking agents, beta-adrenergic blocking agents, angiotensin converting enzyme blockers, antihypertensive agents, sympathomimetics, bronchodilators, xanthines, antihistamines, antitussives, mucolytics, diuretics; carbonic anhydrase inhibitors, urinary alkalinizers, urinary acidifiers, cholinergic stimulants, urolithiasis agents, antiemetic agents, antacids, H2 antagonists, gastromucosal protectants, prostaglandin E1 analogs, proton pump inhibitors, GI antispasmodics/anticholinergics, GI stimulants, digestive enzymes, antidiarrheals, sex hormones, posterior pituitary hormones, oxytocics, adrenal cortical steroids, mineralocorticoids, glucocorticoids, adrenal steroid inhibitors, anti-diabetic agents, thyroid hormones, endocrine/reproductive drugs, prostaglandins, antiparasitics, anticoccidial agents, aminocyclitols, macrolides, penicillins, tetracyclines, lincosamides, quinolones, sulfonamides, antibacterials, antifungal agents, antiviral agents, clotting agents, anticoagulants, erythropoietic agents, blood modifying agents, alkylating agents, antimetabolites; mitotic inhibitors, antineoplastics, and immunosuppresive drugs. Often, the disclosed embodiments of the apparatus, microchemical nanofactory and systems have a morphological structure representative of the compound made. Reagents and conditions useful for making such compounds will be known to a person of ordinary skill in the art of chemical synthesis, and include reagents and conditions published in the chemical literature, or that are used to make compounds in commercial quantities.

### BRIEF DESCRIPTION OF THE DRAWINGS

FIG. 1 is a schematic drawing illustrating general process considerations and exemplary materials that can be made using embodiments of disclosed nanofactories and methods for their use.

FIG. 2 is a schematic drawing illustrating embodiments of hierarchical nanostructures that can be made using embodiments of disclosed nanofactories.

FIG. 3 is a schematic drawing illustrating a moth eye structure that can be made using embodiments of disclosed nanofactories.

FIG. 4 is a schematic drawing illustrating a moth eye structure adjacent a graph of density versus distance from substrate illustrating the density gradient for particular moth eye structures.

FIG. 5 is a schematic diagram of a dendrimer-enabled moth eye structure.

FIG. 6 is a photomicrograph of a ceria film made using nanofactory deposition following sintering at 900° C. for 5 hours.

FIG. 7 is a two-dimensional representation of a dendrimer species.

FIG. 8 is a 3-dimensional, space-filling model of the dendrimer species of FIG. 3.

FIG. 9 is a schematic representation of a fractal plate (providing a geometry similar to the geometry used by Professor Deborah Pence for heat exchanger/desorber applications) illustrating microchannel branching in a device useful for producing dendrimers.

FIG. 10 is a schematic diagram of one embodiment of an interdigital micromixer.

FIG. 10A is a photomicrograph of an interdigital mixer comprising plenums associated with the mixing section.

FIG. 11 is a schematic plan view of one embodiment of an interdigital micromixer with micrometer dimensions.

FIG. 12 is an exploded view of the mixing portion of the interdigital micromixer illustrated in FIG. 11.

FIG. 13 is a schematic view of one embodiment of a nozzle micromixer.

FIG. 14 is a schematic diagram of one exemplary analytical micromixer with a near field scanning optical microscopy (NSOM) probe.

FIG. 15 is a diagram of a continuous reaction system comprising a commercially available micromixer.

FIG. 16 is a schematic perspective diagram of one embodiment of a T mixer.

FIG. 17 is a schematic perspective diagram of one embodiment of a Y mixer.

FIG. 18 is a schematic perspective diagram of one embodiment of a Y mixer.

FIG. 19 is a schematic perspective diagram of one embodiment of a branched mixer.

FIG. 20 is a schematic perspective drawing illustrating one embodiment of a splitting and recombination mixer.

FIG. 21 is a schematic perspective drawing illustrating one embodiment of a collision mixer.

FIG. 22 is a schematic perspective drawing illustrating one embodiment of a superfocusing mixer.

FIG. 23 is a schematic perspective drawing illustrating one embodiment of a serpentine mixer.

FIG. 24 is a schematic perspective drawing illustrating one embodiment of a venturi mixer.

FIG. 25 is a schematic perspective drawing illustrating one embodiment of an active mixer comprising both a mixing section and an adjacent energy focusing section.

FIG. 26 is a schematic plan view of an integrated pneumatic valve from Thorsen, et al. (Thorsen, T., S. J. Maerkl and S. R. Quake. 2002. “Microfluidic Large-Scale Integration,” Science, 298: pp 580-84) showing the ability to extract lower generation dendrons that were not consumed in a prior reaction (excess) from higher generation dendrons.

FIG. 27 is a schematic cross sectional view down the channel of the integrated pneumatic valve illustrated in FIG. 26.

FIG. 28 is a schematic drawing illustrating an ultrasonic welding method for positioning an elastomeric valve layer between other layers in the production of a fluidly actuatable valve prior to welding.

FIG. 29 is a schematic drawing illustrating the system of FIG. 28 after ultrasonic welding.

FIG. 30 is a photomicrograph of a working embodiment of a valve schematically illustrated in FIG. 31 prior to welding.

FIG. 31 is a photomicrograph of a working embodiment of a valve schematically illustrated in FIG. 32 subsequent to welding.

FIG. 32 is a photomicrograph illustrating in cross section a polydimethylsiloxane layer used to implement a valve adjacent a microchannel patterned into a polycarbonate layer.

FIG. 33 is a schematic cross sectional drawing illustrating elastomeric membrane valves in an actuated and unactuated state.

FIG. 34 is a photomicrograph of an array of microchannels in a valve system sealed by a compressed elastomeric layer.

FIG. 35 is a schematic diagram of one embodiment of an extractor.

FIG. 36 is a photomicrographs of one embodiment of a microextractor.

FIG. 37 is a photomicrographs of one embodiment of a microextractor.

FIG. 38 is a photomicrographs of one embodiment of a microextractor.

FIG. 39 is a photomicrograph of one embodiment of a microextractor.

FIG. 40 is a schematic perspective drawing illustrating one embodiment of a sorbent-based or solid phase extraction module.

FIG. 41 is a photomicrograph of a monolithic column prepared using dendrimers.

FIG. 42 is a photomicrograph of a monolithic column prepared using dendrimers.

FIG. 43 is a photomicrograph of a monolithic column prepared using dendrimers.

FIG. 44 is a graph of pore size diameter (μM) versus differential intrusion (mL/g) providing differential pore size distribution profiles of porous polymers prepared using dendrimers template concentrations of 0, 50 (×) and 100 (+) M.

FIG. 45 is a plot of dendrimer concentration (μM) versus plates/m illustrating efficiency as a function of dendrimer template concentration for toluene [columns were prepared using EDMA 16%, total BMA and AMPS 24%, methanol 60%, AIBN 1 wt % (with respect to monomers); dendrimer template concentrations of 0, 50, 100, 200 and 400 μM; data were obtained by applying 20, 25 and 30 kV; the mobile phase was 80% acetonitrile: 20% phosphate buffer 5 mM (pH=7); and UV detection was used at 254 nm].

FIG. 46 is a plot of dendrimer concentration (μM) versus resolution (Rs) illustrating separation resolution of acetone and toluene with respect to dendrimer concentration (where conditions are the same as with FIG. 21).

FIG. 47 illustrates a capillary electrokinetic chromatography CEC separation of lysozyme tryptic digest fragments [separation was performed on a 21 cm (L_{bed}); 31.5 cm (Lₜₒₜ) column prepared using 50 μM dendrimer template; buffer was 40% acetonitrile/60% 40 mM phosphate buffer (pH=2); the applied voltage was 10 kV; and UV detection was used at 200 nm].

FIG. 48 is a photomicrograph of a channel fractured in air.

FIG. 49 is a photomicrograph of a channel in cross section fractured in liquid nitrogen.

FIG. 50 is a photomicrograph of wall-anchored porous polymer monolith.

FIG. 51 is a photomicrograph of wall-anchored porous polymer monolith.

FIG. 52 is an electropherogram (plot of time versus absorbance) illustrating the separation of EDA-core PAMAM dendrimers (G=2, 4 and 5) by capillary zone electrophoresis in phosphate buffer solution.

FIG. 53 is an electropherogram (plot of time versus absorbance) illustrating separations of microreactor-produced dendrimer generation 0.5 from several side products and residual reactants using dendrimer-templated, monolithic sorbents.

FIG. 54 is a schematic perspective drawing illustrating one embodiment of a field flow separator for continuous or batch separation.

FIG. 55 is a schematic perspective diagram of one embodiment of an H-cell useful for separation processes.

FIG. 56 is a schematic perspective drawing illustrating one embodiment of H-cell evaporative separator for continuous or batch separation.

FIG. 57 is a schematic perspective drawing illustrating one embodiment of a liquid-liquid H-cell separator for continuous or batch separation.

FIG. 58 is a schematic perspective drawing illustrating one embodiment of counter current fluid separator for continuous or batch separation.

FIG. 59 is a schematic perspective drawing illustrating one embodiment of a Y-design fluid separator for continuous or batch separation.

FIG. 60 is a schematic perspective drawing illustrating one embodiment of a precipitation separator for continuous or batch separation.

FIG. 61 is a schematic perspective drawing illustrating one embodiment of a membrane filtration separator for continuous or batch separation.

FIG. 62 is a schematic perspective drawing illustrating one embodiment of a size exclusion chromatography separator for batch separation.

FIG. 63 is a schematic diagram illustrating particle flow under positive DEP and particle complement under negative DEP.

FIG. 64 is a schematic plan view illustrating a stacked ratchet DEP comprising an upper electrode-array-defining layer and a lower microchannel array.

FIG. 65 illustrates a microfluidic chip incorporating an inverse fluorescence detector.

FIG. 66 is a block diagram illustrating one embodiment of a laser diode based detection system.

FIG. 67 is a schematic perspective diagram illustrating one embodiment of a modular method for coupling unit operation modules.

FIG. 68 is a schematic perspective diagram illustrating one embodiment of a method for coupling unit operation modules.

FIG. 69 is a schematic diagram illustrating one embodiment of a method for coupling unit operation modules.

FIG. 70 is a schematic diagram illustrating a modular approach to making microchemical nanofactories illustrating an out-of-plane fractal design.

FIG. 71 illustrates modules with integrated heaters and micromixers.

FIG. 72 illustrates an in-line fractal design for compact production of dendrimers (providing a geometry similar to the geometry used by Professor Deborah Pence for heat exchanger/desorber applications).

FIG. 73 is an exploded view of one of the vertices in the fractal device of FIG. 70 having an integrated micromixer, heater and separator.

FIG. 74 is a perspective schematic view of one embodiment of an integrated microchemical nanofactory having an in-plane nozzle micromixer, heating element, and di-electrophoretic separation.

FIG. 75 is a perspective schematic view of one embodiment of an integrated microchemical nanofactory having an out-of-plane, interdigital mixer, heating element, and di-electrophoretic separation.

FIG. 76 is a perspective schematic view of one embodiment of an integrated microchemical nanofactory having an in-plane nozzle micromixer, heating element, and plug flow templated separation.

FIG. 77 is a perspective schematic view of one embodiment of an integrated microchemical nanofactory having an out-of-plane, interdigital mixer, heating element, and plug flow templated separation.

FIG. 78 is a perspective schematic view of one embodiment of a modular microchemical nanofactory.

FIG. 79 is a schematic diagram of a segmented flow reactor adjacent a rotating disk for deposition of functional gradient active nanostructures.

FIG. 80 is a schematic diagram illustrating one embodiment of a photovoltaic cell that can be made according to disclosed embodiments of the present invention.

FIG. 81 is an NMR spectrum of PAMAM G-0.5 synthesized by conventional means in a reaction flask for 3 days.

FIG. 82 is an NMR spectrum of PAMAM G-0.5 synthesized by the continuous reaction system illustrated in FIG. 81.

FIG. 83 is an MS spectra of PAMAM G-0.5 synthesized by the continuous reaction system illustrated in FIG. 15.

FIG. 84 is an MS spectra of PAMAM G0.0 synthesized by the continuous reaction system illustrated in FIG. 15.

FIG. 85 provides ¹H NMR spectra of dendrons G1, G2 and G3 synthesized using embodiments of the disclosed invention.

FIG. 86 provides ¹H-NMR spectra of dendron G1 and dendrimer G1 synthesized using a micromixer according to disclosed embodiments of the present invention.

FIG. 87 provides ¹H-NMR spectra of dendron G2 and dendrimer G2 synthesized using a micromixer according to disclosed embodiments of the present invention.

FIG. 88 is a schematic diagram of one embodiment of a continuous-flow microreactor system used to make metal oxide nanoparticles.

FIG. 89 is a TEM micrograph of a ZnO sample prepared by dipping a copper grid in hot solution collected from a deposition system.

FIG. 90 is an SEM plan view image of an annealed ZnO thin film.

FIG. 91 is an SEM image in cross section of an annealed ZnO thin film made according to embodiments of the disclosed invention, where the thickness of the thin film is about 24 nanometers.

FIG. 92 is an EDS analysis of the annealed ZnO thin film of FIGS. 90 and 91.

FIG. 93 is an XRD spectrum XRD pattern of the annealed ZnO thin film.

FIG. 94 is an estimated bandgap from the optical absorption spectrum of ZnO thin film deposited on a glass slide, and the inset is an optical transmission spectra.

FIG. 95 provides drain current-drain voltage (Ids-Vds) output characteristics with Vgs=−10-40 V in 10 V steps, Vds=0-40 V.

FIG. 96 provides drain current-gate voltage (Ids-Vgs) at Vds=1 V showing a linear extrapolation method for threshold estimation.

FIG. 97 provides Log(Ids)−Vgs transfer characteristics at Vds=40 V.

FIG. 98 is a UV-Vis absorption spectra showing the signature peaks of Au₁₁ core.

FIG. 99 is a TEM image confirming the formation of Au nanoparticles having a uniform size distribution.

FIG. 100 is an SEM image of ceria made using a batch mixer according to Example 4.

FIG. 101 is an SEM image of ceria made using a batch mixer according to Example 4.

FIG. 102 is an SEM image of ceria made using one embodiment of a disclosed nanofactory system and a process according to Example 4.

FIG. 103 is an SEM image of ceria made using one embodiment of a disclosed nanofactory system and a process according to Example 4.

FIG. 104 is a schematic plan view of a linear fractal plate useful for synthesizing dendrimers according to one embodiment of the present invention.

FIG. 105 is a plan schematic view of a portion of the fractal plate of FIG. 104.

FIG. 106 is a cross sectional schematic drawing of a portion of the fractal plate of FIG. 104.

FIG. 107 is a schematic representation of a 5-layered, stacked quarter-half quarter wavelength antireflective coating on a substrate.

FIG. 108 is a plot of reflectance (%) versus wavelength for antireflective coating structure of FIG. 107.

### DETAILED DESCRIPTION

### I. Introduction: Modular and Integrated Nanofactories

Microchemical nanofactories are systems of unit operations implemented within a microchannel format. These nanofactories can be used for a variety of purposes, including dispersing, mixing, synthesizing, manipulating, assembling and/or depositing nano-scale building blocks, including by way of example and without limitation, relatively small scale materials, such as nanoparticles, macromolecules and compounds. For example, working embodiments of disclosed microchemical nanofactories have been used to synthesize nanoparticles and macromolecules. Such nanofactories make use of microreaction technology which takes advantage of the large surface area-to-volume ratios within microchannel structures to accelerate heat and mass transport. This accelerated transport allows for rapid changes in reaction temperatures and concentrations leading to more uniform heating and mixing, which can have dramatic impacts on macromolecular yields and nanoparticle size distributions. Other features of microreaction technology include better defined flow characteristics, narrow residence time distributions, and uniform mass transport. Microreaction technology also allows syntheses of materials in the required volumes at the point-of-use. This eliminates the need to store and transport potentially hazardous materials, and also provides the flexibility for tailoring complex functional nano-materials. Other microchannel unit operations including micromixers, microseparators and microvalves can help to minimize the environmental impact of nanoproduction through solvent free mixing, integrated separation techniques and reagent recycling. Collectively, these microsystems technologies have the potential to transform current batch nanofabrication practices into continuous processes for mass production with precise process control.

Microchemical nanofactories as contemplated herein include modular embodiments, integrated embodiments, and devices and systems that include both modular aspects and integrated aspects. Microchemical nanofactories are used to, for example, precisely mix various reactants, separate out and extract unwanted by-products, detect reaction products in-situ, and continuously vary reactant size, distribution and composition by controlling temperatures, flow rates and concentrations of reactants/media within the microsystem. Thus, nanofactories often include various combinations of unit operations, such as injectors, mixers, reactors, separators, valve(s), detectors and/or extractors. By applying integrated microvalves, the input composition can be rapidly changed and unwanted materials can be extracted. Thin film heaters can be included, such as by sputtering metal onto low to moderate temperature substrates (e.g. polycarbonate). For microscale embodiments of these devices, device features and/or components typically have 1 micrometer to 100 micrometer scale dimensions.

As used herein, “modular” typically refers to unit operations that are performed by separate devices. These devices may be effectively coupled, such as fluidly or electrically coupled, to form a system that functions as a nanofactory. For “modular” devices and systems, separate unit operation can be microscale-type devices. Alternatively, at least one microscale unit operation may be coupled with one or more operations that are not performed on the microscale. For example, the present application discloses embodiments of a method for making dendrimers, as an example of a class of compounds that are advantageously synthesized using a micromixer. While the dendrimers, or dendrons thereof, are advantageously synthesized using a unit operation mixer having microscale features, purification of the compounds made using the device may be best purified “off chip,” such as by using macroscale techniques, such as column chromatography or a centrifugation. Thus, in this example, a modular nanofactory useful for making dendrimers is then coupled with a macroscale purification process/system.

A person of ordinary skill in the art also will appreciate that the micromixing unit operation can be fluidly coupled with an off-chip purification system. Alternatively, products, such as dendrimers, can be made, and then stored off-chip for subsequent purification at a time convenient. Moreover, a person of ordinary skill in the art will further appreciate that off-chip purification is just one example of a modular system. Other unit operations also can be coupled in a modular system. Thus, a first microscale unit operation can be coupled, either affirmatively by physical coupling, or effectively, such as by fluidly coupling, to a separate unit operation, either microscale or macroscale. Moreover, a person of ordinary skill in the art will appreciate that more than two modular unit operations can be coupled together, and that coupled unit operations can be the same or different. For example, two unit operations can be coupled to increase output.

“Integrated” nanofactories typically refers to a device or system where various unit operations are provided on a single “chip”.

Nanofactories also can be coupled to either back end or front end commercial processes. For example, a nanofactory, or factories, could be used to produce a polymeric material, or polymer precursor, that is used in a currently known commercial process. Many such processes require either purchasing the polymer or polymer precursor, shipping the material to the site of use, and then using the material or storing the material on site. Production of such material on site would avoid shipping costs, storage costs, time-dependent degradation of reactants, as such materials could be made on site as needed, etc.

Methods for making exemplary components, exemplary structures of such components, and nanofactories that are modular, integrated, or both, are described in further detail below

### II. Chemical Synthesis

A. Synthesis Generally

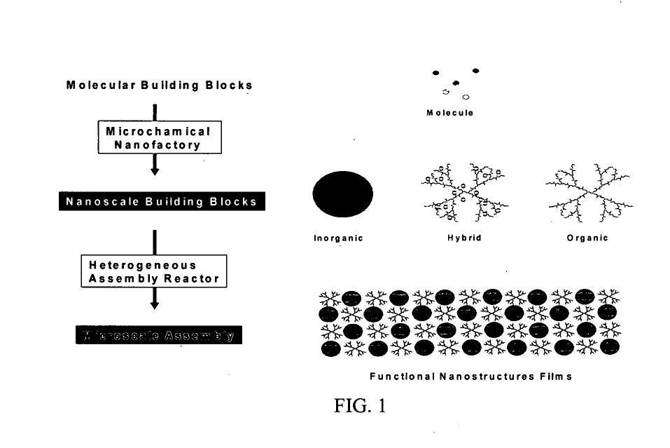

Disclosed embodiments of nanofactories and method for their use can be used to make a multitude of different materials. FIG. 1 schematically illustrates general concepts associated with synthesizing various materials. Molecular building blocks are identified that can be used to make desired materials. These building blocks are then provided to a nanofactory, which is used to make compounds, including inorganic compounds, organic compounds, and combinations thereof, which can be referred to as hybrid chemicals.

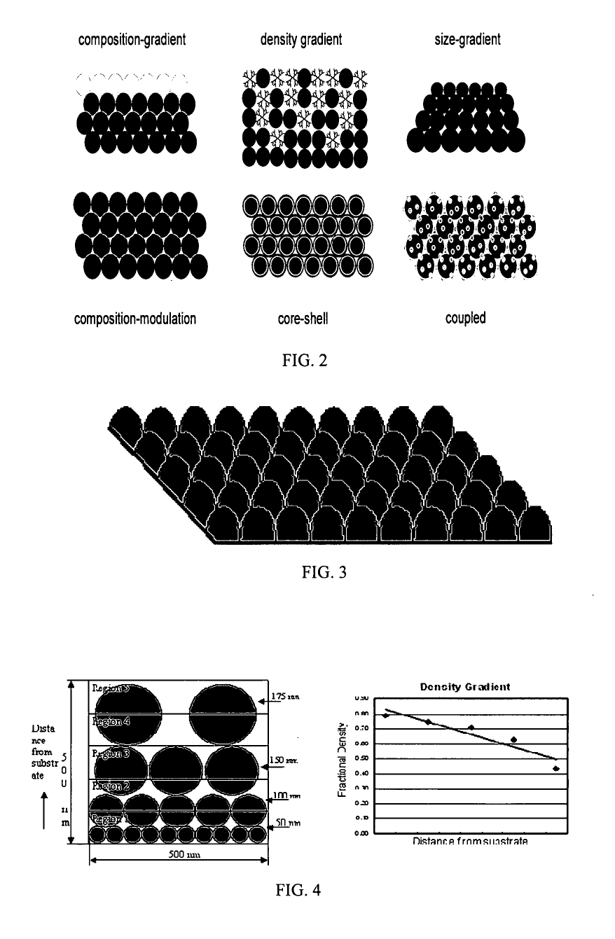

As shown in FIG. 2, disclosed embodiments of microchemical nanofactories could be used to fabricate a variety of tailored hierarchical structures from nano-, micro- to macroscale that are currently impossible/or too cumbersome to produce by other means. For example and without limitation, disclosed embodiments of the invention can be used to make composition gradient structures, density-gradient structures, size-gradient structures, composition modulated structures, core-shell structures, coupled nanoparticle structures, composite films based on core-shell nanoparticles that work actively together in microscale devices, and combinations thereof.

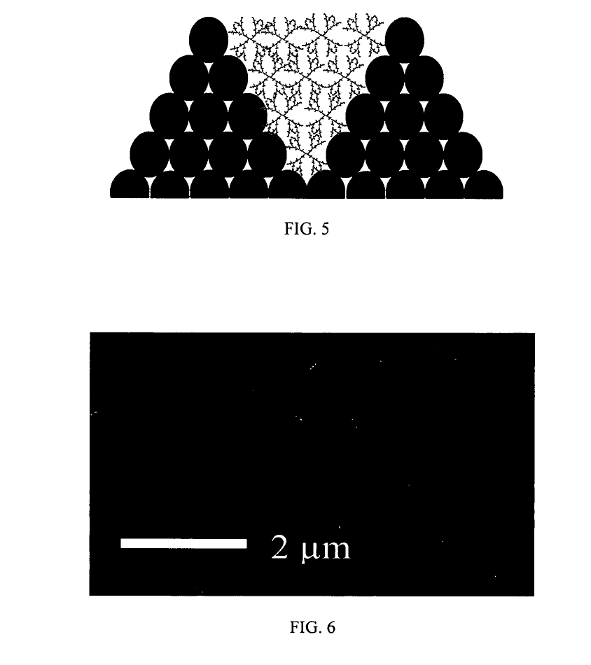

Various different structures are exemplified by further detail concerning moth-eye structures, as illustrated in FIGS. 3-5. Gradient surfaces can be thought to have a low net reflectance based on the destructive interference of an infinite series of reflections at each incremental change in refractive index. One means for producing this gradient is an array of tapered, subwavelength proturbances as shown in FIG. 3. This structure was first reported based on the electron microscopy of the corneas of nocturnal moths by Bernhard who hypothesized that the resultant index gradients were responsible for the reduced eye reflection at night which the moths needed for camouflage. Subsequently, the term “moth-eye” antireflective surface (ARS) has been adopted as describing a tapered array of subwavelength proturbances. FIG. 5 illustrates another mechanism for making suitable moth-eye structures that involves using dendrimers to guide the formation of the desired structure.

FIG. 6 provides another example of a class of materials, inorganic materials, that can be made using disclosed embodiments of the present invention. For example, a metal oxide material can be made using disclosed nanofactory embodiments. FIG. 6 illustrates a ceria film that can be produced using nanofactories, and then deposited on a substrate to form a ceria film. FIG. 6 illustrates ceria nanoparticles deposited to form a film that was sintered at 9000° C. for 5 hours. The superimposed 2 μm scale bar illustrates that the particles formed are nanoparticles.

Microreaction technology can control the formation of both inorganic materials such as quantum dots, as well as organic macromolecules, such as dendritic polymers (dendrimers). Ceramic and metallic nanoparticles also have been produced using microreaction technology. Disclosed embodiments also are directed to depositing smooth, dense and highly-oriented materials, such as nanocrystalline thin films, from a continuous flow microreactor. For example, working embodiments of a microchemical nanofactory have been used to produce functional thin film (40 nm thick) transistors at low temperature (<80° C.) from the solution phase. Disclosed embodiments provide an efficient and low cost route to fabricating small systems by greatly reducing capital equipment and facilities costs compared with conventional micro/nanofabrication processes requiring particle control, vacuum and, many times, high energy inputs.

Based on the above a person of ordinary skill in the art will appreciate that nanofactories and methods for their use can facilitate synthesis of a large number of compounds in addition to dendrimers. Additional examples of particular materials that can be made using disclosed embodiments include, without limitation: oligomers, such as molecular wires [see, for example, James M. Tour, Molecular Electronics, Commercial Insights, Chemistry, Devices, Architecture and Programming, World Scientific, which is incorporated herein by reference], rods [see, for example, Peter F. Schwab, Michael D. Levin and Josef Michl in Molecular Rods, Chem. Rev., 99, 1863 (1999), incorporated herein by reference], oligoenes, oligoynes, oligoenynes, oligoarylenes, oligonacenes, oligoarylenevinylenes, oligothiophene, oligotetrathiafulvalenes, oligoanilines, oligopyrroles [Electronic Materials: The Oligomer Approach, Edited by K. Mullen and G. Wegner, Wiley-VCH, (1998); biological macromolecules, such as polypeptides, proteins, enzymes, antibodies, fibrous proteins, globular proteins, membrane proteins, glycoproteins; polysaccharides, oligosaccharides: e.g. hyaluronic acid, heparin, chitin, celluose, amylose; nucleic acids, polynucleotides, oligonucleotides: DNA, RNA, including DNA or RNA analogs oligonucleotides (e.g. antisense oligonucleotides); simple and complex natural products (such as taxol) and mimics of natural products; molecules having useful 3-dimensional shapes, such as supermolecules, one example of which is diamondoid, that typically are constructed using molecular building blocks, such as described by Damian G. Allis and James T. Spencer, Handbook of Nanoscience, Engineering, and Technology, 16 (2003), incorporated herein by reference; commercial polymeric materials, now known or hereafter developed, particularly those with oft repeated monomers molecular blocks; respiratory stimulants such as doxapram; analgesics such as meperidine, buprenorphine, acetaminophen; nonsteroidal anti-inflammatory/analgesic agents such as aspirin, ketoprofen, naproxen; behavior-modifying agents such as amitriptyline, imipramine, clomipramine; tranquilizers/sedatives such as diazepam, midazolam, xylazine; anesthetic agents such as pentobarbital, propofol, ketamine; reversal agents such as naloxome, yohimbine, neostigmine; anticonvulsants such as phenobarbital, phenyloin, primidone; muscle relaxants such as methocarbamol, succinylcholine, dantrolene; inotropic agents such as digoxin, digitoxin, dobutamine; antiarrhythmic drugs such as lidocaine, mexiletine quinidine; anticholinergics such as atropine and glycopyrrolate; ACE inhibitors such as benazepril, captopril and enalapril; calcium channel blocking agents such as amlodipine, diltiazem, verapamil; vasodilating agents such as hydralazine, isoxsuprine, nitroglycerine; alpha-adrenergic blocking agents such as phenoxybenzamine, prazosin; beta-adrenergic blocking agents such as atenolol, esmolol, propranolol; angiotensin converting enzyme blockers such as captopril, enalapril; antihypertensive agents such as nitroprusside; bronchodilators, sympathomimetics such as albuterol, clenbuterol, terbutaline; bronchodilators, xanthines such as aminophylline, theophylline; antihistamines such as doxepin, hydroxyzine, pyrilamine; antitussives such as codeine, butorphenol, hydrocodone; mucolytics such as acetylcysteine; diuretics; carbonic anhydrase inhibitors such as acetazolamide, dichlorphenamide; diuretics; thiazide diuretics such as chlorothiazide, hydrochlorothiazide; loop diuretics such as furosemide, ethacrynic acid; potassium sparing diuretics such as spironolactone; osmotic diuretics such as glycerin, mannitol; agents for urinary incontinence/retention such as ephedrine, oxybutynin, phenoxybenzamine; urinary alkalinizers such as sodium bicarbonate; urinary acidifiers such as methionine, ammonium chloride; cholinergic stimulants such as bethanechol; agents for urolithiasis such as ammonium chloride, methionine, allopurinol; antiemetic agents such as chlorpromazine, meclizine, metoclopramide; antacids such as aluminum gels, calcium salts, sodium bicarbonate; H2 antagonists such as cimetidine, famotidine, ranitidine; gastromucosal protectants such as sucralfate; prostaglandin E1 analogs such as misoprostol; proton pump inhibitors such as omeprazole; GI antispasmodics/anticholinergics such as aminopentamide, isopropamide, propantheline; GI stimulants such as cisapride, dexpanthenol, neostigmine; digestive enzymes such as pancrelipase; antidiarrheals such as diphenoxalate/atropine, bismuth subsalicylate, clioquinol; sex hormones such as estradiol, altrenogest, stanozolol; posterior pituitary hormones, including vasopressin agents such as desmopressin; oxytocics such as oxytocin; adrenal cortical steroids such as corticotrophin-ACTH; mineralocorticoids such as desoxycorticosterone piv, fludrocortisone; glucocorticoids such as dexamethasone, hydrocortisone, prednisone; adrenal steroid inhibitors such as mitotane, selegiline; anti-diabetic agents such as insulin, chlorpropamide, glipizide; thyroid hormones such as levothyroxine, liothyronine, TSH; miscellaneous endocrine/reproductive drugs such as bromocriptine, chorionic gonadotropin-HCG, follicle stimulating hormone; prostaglandins such as cloprostenol, dinoprost, fluprostenol; antiparasitics such as fenbendazol, ivermectin, praziquantal; anticoccidial agents such as amprolium, decoquinate; antibiotics, aminocyclitols such as amikacin, gentamycin, neomycin; cephalosporins such as cefazolin, cephalothin, ceftiofur; macrolides such as erythromycins, tylosin; penicillins such as amoxicillin, ticarcillin, carbenicillin; tetracyclines such as doxycycline, tetracycline, oxytetracycline; antibiotics, lincosamides such as clindamycin, lincomycin, tilmicosin; quinolones such as enrofloxacin, ciprofloxacin, orbifloxacin; sulfonamides such as sulfadimethoxine, sulfamethoxazole/trimethoprim, sulfadiazine/trimethoprim; miscellaneous antibacterials such as chloramphenicol, clioquinol, metronidazole; antifungal agents such as itraconazole, ketoconazole, amphotericin B; antiviral agents such as acyclovir, interferon alfa-2A; clotting agents such as phytonadione, protamine sulfate, aminocaproic acid; anticoagulants such as aeparin, warfarin; erythropoietic agents such as epoetin alfa, ferrous sulfate, iron dextran; miscellaneous blood modifying agents such as hemoglobin glutamer-200, pentoxifylline; antineoplastics/immunosuppresives; alkylating agents such as chlorambucil, cisplatin, cyclophosphamide; antimetabolites such as cytarabine, methotrexate, thioguanine; antibiotics such as bleomycin, doxorubicin; mitotic inhibitors such as vinblastine, vincristine; miscellaneous antineoplastics such as asparaginase, piroxicam, hydroxyurea; and immunosuppresive drugs such as azathioprine, cyclophosphamide, mercaptopurine.

B. Dendrimers

Substantial experience has been gained from making dendrimers using modular microreactors. Thus, the following description exemplifies certain features of disclosed embodiments with reference to a microsystem for synthesizing dendrimers.

Throughout nature and at all scales there are examples of the interrelationship between shape and function. Control of structure on the nanometer/macromolecular scale is therefore of great interest in chemistry and engineering. The present invention uses the exquisite three-dimensional structural motifs and startling reproducibility and monodispersity demonstrated in macromolecules as an inspiration for a unique approach to the production of highly ordered and structurally elegant molecules, such as dendritic macromolecules, or dendrimers. Microreactor-based dendrimer production within fractal nanofactories demonstrates the ability to control the hundreds of parallel reactions necessary to economically produce highly ordered dendrimers.

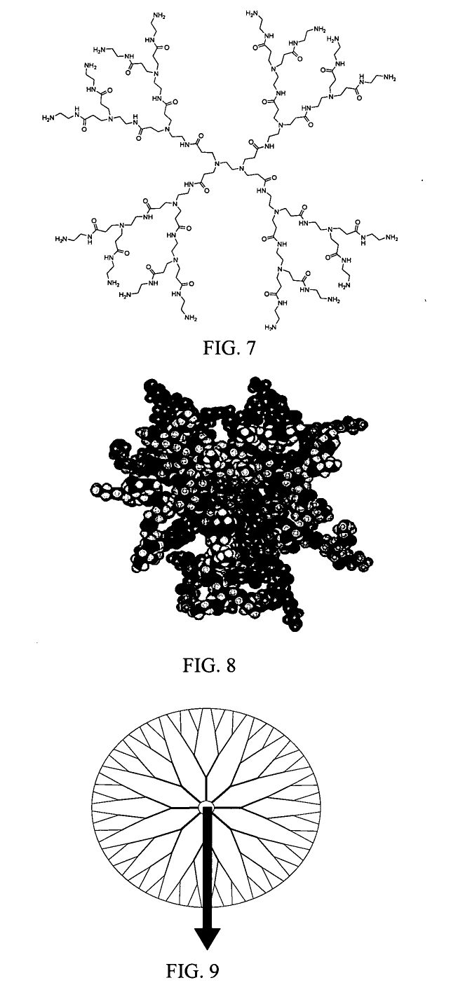

Dendrimers are highly-branched, nanometer-sized molecules with symmetrical fractal morphologies. FIG. 7 provides a chemical structural formula of a representative dendrimers, and FIG. 8 provides a space-filling model of the dendrimers of FIG. 7. The word dendrimer is derived from the Greek words dendri (branch, tree-like) and meros (part of). Dendrimers consist of a core-unit, branching units, and end groups located on their peripheries. Their dendritic architecture presents great potential for a wide variety of applications. Dendrimers hold great promise as building blocks for complex supramolecular structures and as nanoscale carrier molecules in drug delivery, where nanoparticles and nanocapsules are gaining popularity. The molecules can be assembled with startling precision, a necessity when the goal is construction of nanoscale structures or devices with sophisticated and complex functionality. The limiting factor to fully realize the potential applications of dendrimers is production cost. For dendrimers to realize their full potential, methods must be developed by which the uniformity and efficiency of nature can be closely approximated in the production of these macromolecules. Current methods of production suffer from low yields, high costs and the inability to produce large, higher generation dendrimers. The high rates of heat and mass transfer found in microchannels will provide the basis for greater control of dendrimer synthesis enabling the low-cost production of larger dendrimers while providing an opportunity to explore the fundamentals of their reaction kinetics. The conformation of dendritic macromolecules can be manipulated through micro- and nanofluidic mixers and novel microseparations techniques to minimize excess reagent and defective product to further improve yields in downstream reactions.

For the branched dendrimers, a fractal nanofactory (such as might employ the fractal plate of FIG. 9) has a strong resemblance to the morphology of the compound made by the device. Third, dendrimers can be made by an iterative synthesis process, which is amenable to chemical synthesis by a nanofactory since the reactions are repetitive, the separation techniques all can be similar as the molecules produced by the reactions are similar, a limited number of reagents need be mixed, etc.

A critical barrier to the routine use of dendrimers is the tedious, expensive means of their synthesis. This synthesis consists of two constantly repeating reaction steps involving: 1) coupling a central unit to two branching units; and 2) activating the branches so they can react further. Two general approaches (divergent and convergent) to dendrimer synthesis exist. Divergent synthesis starts from a seed and progresses towards the periphery of the dendrimer, while convergent synthesis proceeds from the periphery to a core.

1. Divergent Synthesis

The divergent approach, arising from the seminal work of Tomalia and Newkome, initiates growth at the core of the dendrimer and continues outward by the repetition of coupling and activation steps. In divergent synthesis, several hundred reactions may be required to obtain five or six dendrimer generations (sizes of interest). In this case the yield for each step multiplies through to determine the total yield. For example, in the synthesis of a fifth generation poly (propylene imine) dendrimer (64 imine groups; 248 reactions), a yield of 99% per reaction will result in only 0.99²⁴⁸=8.27% of defect-free dendrimer. To further complicate matters, the similar sizes of defective and defect-free dendrimers then make separation difficult. Exponential growth in the number of reactions to be performed to produce higher generations makes divergent synthesis an unlikely method for the production of uniform dendrimers beyond generation five or six unless the yield at each step exceeds 99.8%. In addition, extremely excessive amounts of reagents are required in latter stages of growth to reduce side reactions and force reactions to completion. This not only increases the cost but also causes difficulties in purification.

2. Convergent Synthesis

Convergent synthesis initiates growth from the exterior of the molecule, and progresses inward by coupling end groups to each branch of the monomer. The single functional group at the focal point of the wedge-shaped dendritic fragment can be activated after the coupling step. Coupling the activated dendrons to a monomer creates a higher generation dendron. Finally, the globular, multi-dendron dendrimer is generated by attaching the dendrons to a polyfunctional core. Here, a small and constant number of reaction sites are maintained in each reaction step. Consequently, only a small number of side products are possible in each step. As a result, the reactions can be driven to completion with only a slight excess of reagent and defective product can be eliminated prior to subsequent reaction. Thus, convergent synthesis has the potential to produce purer dendrons and dendrimers than divergent synthesis. Furthermore, the ability to precisely place functional groups throughout the structure, to selectively modify the focal point, and to prepare well-defined asymmetric dendrimers make the convergent approach attractive. However, since the coupling reaction occurs only at the single focal point of the growing dendron, the preparation of higher generation dendrons and dendrimers (typically above the sixth generation) is sterically hindered, resulting in decreased yields. This is especially evident in the reaction between high generation dendrons and the core. This drawback has limited the commercialization of dendrimers produced by convergent synthesis. Embodiments of the nanofactories disclosed in the present application address this drawback.

Hence, dendrimers were selected for initial synthesis using embodiments of the disclosed nanofactories for several reasons. First, dendrimers are a genus of useful compounds. Second, the nanofactories may be particularly suitable for making compounds where the structure of the device is representative of the morphology of the end product made using the device. For the branched dendrimers, the fractal nanofactory has a strong resemblance to the morphology of the compound made by the device. Third, dendrimers can be made by an iterative synthesis process, which is amenable to chemical synthesis by a nanofactory since the reactions are repetitive, the separation techniques all can be similar as the molecules produced by the reactions are similar, a limited number of reagents need be mixed, etc.

### III. Microlamination

A. General Discussion

Microchemical nanofactories, and/or individual components thereof, can be made by microlamination technology. Microlamination methods are described in several patents and pending applications commonly assigned to Oregon State University, including U.S. Pat. Nos. 6,793,831, 6,672,502, and U.S. patent applications, Nos. 60/514,237, entitled High Volume Microlamination Production Of Mecs Devices, and 60/554,901, entitled Microchemical Microfactories, all of which are incorporated herein by reference.

Microlamination consists of patterning and bonding thin layers of material, called laminae, to generate a monolithic device with embedded features. Microlamination involves at least three levels of production technology: 1) lamina patterning, 2) laminae registration, and 3) laminae bonding. Thus, the method of the present invention for making devices comprises providing plural laminae, registering the laminae, and bonding the laminae. The method also may include dissociating components (i.e., substructures from structures) to make the device. Component dissociation can be performed prior to, subsequent to, or simultaneously with bonding the laminae.

Disclosed embodiments of a method for making microlaminated devices also may include the deposition and coating of various materials onto laminae or within post-bonded microchannels for a variety of purposes including catalysis, increasing or decreasing hydrophobicity, passivation, thin film heaters/sensors, etc. For example, silica can be deposited on laminae, or microstructures defined thereon, to “passivate” the structural material, that is to increase its compatibility with the processes for which the device is designed. As another example, a metal, metals, alloy or alloys, such as NiB, could be deposited onto structural materials used to form the laminae, such as stainless steel, for various purposes, including facilitating subsequent brazing. Coating may occur prior to laminae registration, after registration, but before bonding, or after bonding.

In one aspect of the invention, laminae are formed from a variety of materials, particularly metals, alloys, including intermetallic metals and alloys, polymeric materials, including solely by way of example and without limitation, PDMS, polycarbonates, polysulfones, polyimides, etc., ceramics, and combinations of such materials. The proper selection of a material for a particular application will be determined by other factors, such as the physical, chemical, thermal or mechanical properties of the metal or metal alloy and cost. Examples of metals and alloys particularly useful for metal microlamination include stainless steel, carbon steel, phosphor bronze, copper, graphite, and aluminum.

Laminae useful for the microlamination method of the present invention can have a variety of sizes. Generally, the laminae have thicknesses of from about 1 mil to about 32 mils thick, preferably from about 2 mils to about 10 mils thick, and even more preferably from about 3 to about 4 mils thick (1 mil is 1 one-thousandth of an inch). Individual lamina within a stack also can have different thicknesses.

B. Fabricating Laminae

1. Lamina Patterning

Lamina patterning may comprise any one or a combination of the myriad machining, molding or forming techniques used to fabricate a micro- or macro-scale pattern in the lamina. The pattern produced depends on the device being made. Without limitation, techniques for machining or etching include laser-beam, electron-beam, ion-beam, electrochemical, electrodischarge, chemical and mechanical material deposition or removal can be used. The lamina can be fabricated by both lithographic and non-lithographic processes. Lithographic processes include micromolding and electroplating methods, such as LIGA, and other net-shape fabrication techniques. Some additional examples of lithographic techniques include chemical micromachining (i.e., wet etching), photochemical machining, through-mask electrochemical micromachining (EMM), plasma etching, as well as deposition techniques, such as chemical vaporization deposition, sputtering, evaporation, and electroplating. Non-lithographic techniques include electrodischarge machining (EDM), mechanical micromachining, laser micromachining (i.e., laser photoablation), hot embossing, and injection molding. Photochemical machining of metals and hot embossing of polymers are preferred for mass-producing devices.

A currently preferred method for patterning lamina patterns for prototyping devices is laser micromachining, such as laser numerically controlled micromachining. It is particularly well suited for through-cutting polymers as it leaves little if any residue or burr. Laser micromachining has been accomplished with pulsed or continuous laser action in working embodiments. Machining systems based on Nd:YAG and excimer lasers are typically pulsed, while CO₂ laser systems are continuous. Nd:YAG systems typically were done with an Electro Scientific Industries model 4420. This micromachining system used two degrees of freedom by moving the focused laser flux across a part in a digitally controlled X-Y motion. The laser was pulsed in the range of from about 1 kHz to about 10 kHz. This provides a continuous cut if the writing speed allows pulses to overlap. The cutting action is either thermally or chemically ablative, depending on the material being machined and the wavelength used (either the fundamental at 1064 nm, the second harmonic at 532 nm, the third harmonic at 355 nm or the fourth harmonic at 266 nm). The drive mechanism for the Nd:YAG laser was a digitally controlled servo actuator that provides a resolution of approximately 2 μm. The width of the through cut, however, depends on the diameter of the focused beam.

Laminae also have been machined with CO₂ laser systems. Most of the commercial CO₂ lasers semi-ablate or liquefy the material being cut and because of the generally higher powers, may through-cut laminae in a single pass. A high-velocity gas jet often is used to help remove debris. As with the Nd:YAG systems, the laser (or workpiece) is translated in the X-Y directions to obtain a desired pattern in the material. Because of their inherently larger beam waists, CO₂ lasers typically are not as precise as other lower wavelength lasers.

An Nd:YAG pulse laser has been used to cut through, for example, 90-μm-thick steel shims. The line widths for these cuts were approximately 35 μm wide, although with steel, some tapering was observed. For the 90-μm-thick sample, three passes were made using 1 kHz pulse rate, an average laser power of 740 mW, and a distance between pulses of 2 μm. Also, the cuts were made at 355 nm. Some debris and ridging was observed along the edge of the cut on the front side. This material was easily removed from the surface during lamina preparation, such as by surface polishing.

Patterns also have been fabricated in laminae using a CO₂ laser. For example, a serpentine flexural spring used in a miniature Stirling cooler has been prepared using a CO₂ laser. The CO₂ through-cuts were approximately 200 μm wide and also exhibited a slight taper. The width of the CO₂ laser cut was the minimum achievable with the system used. The part was cleaned in a lamina preparation step using surface polishing to remove debris.

Pulsed Nd:YAG lasers also are capable of micromachining laminae made from polymeric materials, such as laminae made from polyimides. Pulsed Nd:YAG lasers are capable of micromachining these materials with high resolution and no debris formation. Ultraviolet wavelengths appear best for this type of work where chemical ablation apparently is the mechanism involved in removing material. Clean, sharp-edged holes in the 25-50 μm diameter range have been produced.

2. Lamina Preparation

In another aspect of the invention, lamina fabricating includes lamina preparation. The laminae can be prepared by a variety of techniques. For example, surface brushing, lapping, polishing or electrochemical deburring of a lamina following patterning may be beneficial. Moreover, acid etching can be used to remove any oxides from a metal or alloy lamina. In one embodiment of the invention, lamina preparation includes applying an oxide-free coating to some or all of the laminae. An example of this would be electroplating gold onto the lamina to prevent oxidation at ambient conditions. In another case, it may be useful to provide a thin film or electroplated coating to aid the bonding process. For example, sputtered silica can be used to facilitate bonding of many different polymeric, metallic and ceramic materials with PDMS. It also may be beneficial to flatten laminae such as with a mechanical press or a vacuum hot press or perhaps by deforming the lamina in tension. Cleaning also may include using common degreaser(s) and/or residue remover(s).

In another embodiment of the invention, lamina preparation includes filling the spaces between the structures and substructures with a material, referred to herein for convenience as a fixative, that holds the structure and substructure together before bonding the laminae and after the fixture bridges are eliminated. For instance, investment casting wax can be used as the fixative to hold together the structure and substructure. The fixture bridges are then eliminated, and the substructure is maintained in contact with the structure by the fixative. The fixative is eliminated during or after bonding the laminae together, thus dissociating the substructure from the structure.

C. Laminae Registration

Laminae registration comprises (1) stacking the laminae so that each of the plural lamina in a stack used to make a device is in its proper location within the stack, and (2) placing adjacent laminae with respect to each other so that they are properly aligned as determined by the design of the device. It should be recognized that a variety of methods can be used to properly align laminae, including manually and visually aligning laminae.

The precision to which laminae can be positioned with respect to one another may determine whether a final device will function. The complexity may range from structures such as microchannel arrays, which are tolerant to a certain degree of misalignment, to more sophisticated devices requiring highly precise alignment. For example, a small scale device may need a rotating sub-component requiring miniature journal bearings axially positioned to within a few microns of each other. Several alignment methods can be used to achieve the desired precision. Registration can be accomplished, for example, using an alignment jig that accepts the stack of laminae and aligns each using some embedded feature, e.g., corners and edges, which work best if such features are common to all laminae. Another approach incorporates alignment features, such as holes, into each lamina at the same time other features are being machined. Alignment jigs are then used that incorporate pins that pass through the alignment holes

Thermally assisted lamina registration also can be used as desired. Thermally-assisted edge alignment can register laminae to within 10 microns, assuming the laminae edges are accurate to this precision. Additional detail concerning thermally assisted lamina registration is provided by copending application No. 60/514,237, which is incorporated herein by reference. Alternatively, laminae may be aligned by using self-aligning, nested features that can be produced in the laminae during the patterning step. Typically, these features are easier to implement for molding or forming applications, such as injection molding or embossing. They typically require using blind and raised features on respective features of the laminae.

Greater levels of registration precision can be achieved by incorporating compliant mechanisms into either the fixture or the laminae themselves. In particular, laminae have been designed with edge springs that can be used to align laminae at room temperature and absorb any differential thermal expansion with increasing temperature. Layer-to-layer registration below 5 micrometers has been achieved using this method on 50 mm scale devices. Also, for certain bonding processes such as ultrasonic welding, it has been found that the use of functional features such as half moon channels in conjunction with small wires, tubes or cylindrical objects that fit within the channel formed by two half moon channels on adjacent laminae can give even greater levels of registration precision.

For example, in a dialysis application, the width of the microchannel may be small, on the order of the microchannel height, to minimize the span of the membrane since the membrane is not stiff. In this application, the registration of stiff laminae would be important since a misregistration of 100 μm could be as large as the width of the channel itself and could significantly impact mass transfer across the membrane. However, in some heat exchanger applications, the use of metal laminae as a heat transfer surface between two channels warrants much larger spans yielding much wider channels. Under these circumstances, layer-to-layer registration of laminae may not be very important since a 100 μm misregistration would impact only a small fraction (<1%) of the heat transfer surface. Generally, the efficiency of the heat and mass transfer across the fin or membrane is inversely proportional to the registration between laminae divided by the width of the microchannel.

In the diffusion bonding of metal laminae, previous studies have shown various methods that can be used for registering laminae during the diffusion bonding cycle. One method called thermally-enhanced edge registration (TEER) uses the differential thermal expansion between fixture and laminae to cause a registration force to be exerted on the laminae at high temperature. However, these methods have been found to be highly sensitive to the dimensional tolerances of the laminae and fixture leading to either misregistered (due to no interaction with the fixture) or warped laminae. Further, even by loosening up tolerances through the use of compliant fixture pins, TEER methods have been found to cause local deformation adjacent to the pin which results in no registration force and no improvement in registration. It is expected this is caused by a weakening of the mechanical properties of the lamina and perhaps a rise in the friction between laminae associated with the temperature increase.

More recently, efforts have been made to develop integral springs that can be designed into laminae to provide registration forces at room temperature. These techniques are suspected to provide better and more reliable registration tolerances. These techniques allow for the differential expansion of the laminae and fixture by providing compliance, and therefore dampening of registration forces, at high temperatures. Previous studies involving integral springs have shown that the quality of registration is influenced by at least two factors: 1) the friction between laminae; and 2) the magnitude of the registration force. With regard to friction between laminae, studies using these integral springs have found that each individual lamina must be flat (4 μm over 1 cm) and burr-free (<<1 μm) in order to achieve consistent layer-to-layer registration below 10 μm over 25 mm devices. Previous studies have also found that moderate registration forces (7 N) resulted in better registration than either low (0.5 N) or high registration forces (11 N). At small registration forces (registration-force-to-lamina-weight [force-to-weight] ratio=0.5/0.01=50), the lamina misalignment was found to be poor simply because it was unable to overcome any friction between the laminae and the registration pins. At high registration forces (force-to-weight ratio>1000), the misalignment was also found to be poor and it was observed that the laminae elastically buckled. It is expected that this out-of-plane buckling was a chief source of the misregistration. The best registration was found at a force-to-weight ratio of 700. At this level, the laminae did not buckle. Based on prior results, it is expected that registration of laminae is impacted largely by: 1) surface friction between laminae caused by large surface asperities (e.g. burrs, warpage, etc.); and 2) the buckling phenomena of the laminae.

The main function of a registration fixture is to register individual laminae with respect to a datum reference frame by eliminating all degrees of freedom on the laminae, generally, three translations and three rotations. Assuming that the X-Y plane is the flat surface of the fixture/laminae and +Z direction goes upward, three degrees of freedom are eliminated by placing a lamina on the surface of the fixture: two rotations and one translation. Specifically, the rotations about the X and Y axes and the translation along the Z axis is fixed. Further constraint of the remaining three degrees of freedom (Z rotation, X translation and Y translation) are accomplished with the use of three additional fixture pins. In order to fully constrain the laminae for registration, three additional pins were needed opposite the original three pins. These pins provide the registration force to hold the laminae against the datum lines. TEER methods have been found to be inconsistent and hard to control.

An alternative approach is to allow the laminae to comply when in contact with the fixture at room temperature. This can done using an integral spring implemented by making a small slot near the edge of the laminae to be registered. Assuming that the laminae was inserted into the fixture at an angle at room temperature, alignment pins would restore the misplaced lamina with respect to the fixture datums. The friction between adjacent laminae/fixture surfaces was assumed to be negligible based on the light weight and burr-free surface of a thin stainless steel shim. The main resistance to the restoring forces is assumed to be the friction between the lamina and the pins. interfacing fixture pins. In this model, N represents the normal force, f represents the friction at the pin contact surface, D represents the overall dimension of the lamina and L represents the distance between the center of the lamina and the restoring alignment pin. Assuming that the initial angle of misregistration is small (<5°), then all pin reaction forces are assumed to be perpendicular to the edges of the lamina. Under these conditions, the FBD clearly shows that pin frictions (f₁, f₂, f₃ and f₄) create a counterclockwise momentum against the registration force (N₁ and N₄). Therefore, in order to have self alignment behavior, the registration moment must be higher than the friction moment.

$$\sum {M}_{Center} ; 2 \cdot N \cdot L \geq 4 \cdot f \cdot ( \frac{D}{2} )$$

$$2 \cdot N \cdot L \geq 4 \cdot μ \cdot N \cdot ( \frac{D}{2} )$$

$$\frac{L}{D} \geq μ$$

Based on this analysis, the ratio L/D must be greater than the coefficient of static friction between the pin and the laminae, or the laminae will not self register. L/D cannot be greater than 0.5 based on geometry so the friction coefficient at the surface contact must be less than 0.5. The common value of the coefficient of static friction of polish tungsten (pins) and polished stainless steel (laminae) is around 0.3. This suggests that the distance from the location of the pin to the centerline of the shim cannot be less than about ⅓ of the major dimension of the device on that side.

Further, if one of the two pin sides was reduced to a single pin, the system would lose one registration force (e.g. N₁) for rotating the lamina. The summation of the moments would be:

$$\sum {M}_{Center} ; N \cdot L \geq 4 \cdot f \cdot ( \frac{D}{2} )$$

$$N \cdot L \geq 4 \cdot μ \cdot N \cdot ( \frac{D}{2} )$$

$$\frac{L}{D} \geq 2 \cdot μ$$

As a result, the registration moment would be cut in half and, therefore, the maximum friction coefficient allowed would reduce to only 0.25. Consequently, the additional pin is required for the fixture.

As mentioned previously, the purpose of the registration force is to hold the lamina against the datum references. The registration force must be large enough to overcome the friction between the laminae and the pins but small enough to not buckle or otherwise yield the laminae. Further, too small of a force could lead to poor shim alignment. This section will discuss guidelines for setting the registration force.

For a given integral spring, the registration force can be controlled by adjusting the dimensions of the spring and the amount of displacement. For simplicity sake, consider a cantilever spring with a force-displacement curve that follows the basic cantilever beam deflection formula:

$${δ}_{max} = - \frac{P \cdot {L}^{3}}{3 \cdot E \cdot I} \qquad ( 1 )$$

where δₘₐₓ is the maximum deflection, L is the span (or length) of the spring, E is young's modulus, P is the normal force applied at the end of the beam, and I is the area moment of inertia of the beam cross-section. For a rectangular cross-section, the area moment of inertia is:

$$I = \frac{b \cdot {h}^{3}}{12} \qquad ( 2 )$$

where h is the width (or height) of the spring along the axis of the load and b is the breadth of the spring transverse to the load. Assuming that laminae registration springs follow this behavior, the two main parameters that determine the characteristics of the spring are: 1) the span; and 2) the width. Based on a spring design, the registration force at room temperature could be controlled by changing the spring displacement based on changing the amount of interference between the laminae and the fixture. In general, it is desirable to keep the spring displacement as small as possible while allowing for any patterning tolerances. As an example, if laser machining accuracy can be held to approximately 10 μm, a minimum initial displacement of at least 20 μm might be used to insure registration repeatability.

In order to satisfy all of the conditions mentioned above, a method of designing the spring by iteration was implemented. The requirement was to determine the elastic and buckling limits of a particular spring design and then determine if the maximum displacement in the limiting condition (either yielding or buckling) would provide adequate registration force (force-to-weight ratio between 100 and 1000). A standard spreadsheet for use in designing the spring was developed. The first step was to input all of the spring parameters including the shim material properties into the spreadsheet. The maximum registration force and maximum displacement were calculated based on the yield point of the material. If the maximum displacement was smaller than the minimal displacement (e.g. 20 μm), the spring parameters were adjusted (particularly the span and width of the spring).

After the rough dimension of the spring was determined by yield, the next step was to determine the maximum registration force and maximum displacement based on the critical buckling load. At room temperature, the shim should not buckle in order to have a rigid datum reference. Because the geometry of the shim was very complex, basic beam theory could not be applied. A finite element analysis (FEA) was used to determine the critical buckling load for the lamina geometry and then that value used to determine the maximum spring displacement. Again, if the maximum spring displacement was less than the patterning accuracy, the spring dimension was readjusted. These steps were iterated until all of the requirements were satisfied.

Different trials were attempted to determine what level of accuracy could be achieved using a spring designed using the method described above. The test article was a five microchannel device using flat, through-cut 178 μm (0.007″) stainless steel. Test articles were fabricated using laser machining, hand deburring and diffusion bonding. First, the shimstock was patterned using the ESI 5330 laser (355 nm) via drilling system. Next, the twelve layers of laminae were individually deburred by hand and placed into the designed graphite fixture. To reduce the pin friction, a barrier film of boron nitride was sprayed onto all pins and surfaces on the fixture. After stacking, the fixture was put into a vacuum hot press and diffusion bonded at 800° C. for two hours. The resultant monolithic device was then cut open using a metallographic saw to observe the average misalignment. This particular shim design was capable of producing four devices at a time. The large oval slots were integrated into the design to provide guidance for singulation. The final dimension of the registration spring was 20 mm in length and 2 mm in width. The smaller oval slots adjacent to the integral springs were used to spread out the distribution of pressure throughout the laminae. This design was arrived at through the use of FEA. The ratio of L/D was 0.46. The size of the lamina was 5.38 cm. (2.12″). Between minimum and maximum displacement, this spring was capable of providing from 7 to 30 N of registration force prior to buckling or yielding.

Using this design with a designed interference of 20 μm, the misalignment was found to be exceptionally low with a peak-to-peak misalignment of 8.1 μm. This would translate into an approximate, average misalignment below 3 μm layer-to layer. Visually, the channel height variation due to warpage was also exceptionally low. It is expected that this result may be dependent on the protocol for loading the laminae with shims in which it is important to not permanently deform the laminae upon loading.

The average misalignment was much lower than in previous trials. This level of misalignment suggests that the bulk of this error is likely due to the tolerance limit of the laser machining process (i.e. the laser machining has a general accuracy of 10 μm) or perhaps the measurement error of the optical microscope (about 2 μm). Further improvements (lower misalignment) will require better patterning and characterization accuracies.

In all, this analysis suggests that concepts useful for designing alignment systems based on integral springs include: (1) using a small spring deflection to overcome the patterning tolerances (e.g. 2× the machining tolerance) while not exceeding the elastic limit or critical buckling load; (2) controlling the registration force using the span and width of the spring; (3) creating a fixture with enough contact points to constrain all degrees of freedom and with an L/D ratio in excess of the static coefficient of friction between the laminae and pins; and (4) minimizing the coefficient of static friction of all contact surfaces.

D. Laminae Bonding

Laminae bonding comprises bonding the plural laminae one to another to produce a monolithic device (also referred to as a laminate). Laminae bonding can be accomplished by a number of methods including, without limitation, diffusion soldering/bonding, thermal brazing, adhesive bonding, thermal adhesive bonding, curative adhesive bonding, electrostatic bonding, resistance welding, microprojection welding, laser transmission welding, microwave welding, infrared welding, ultrasonic welding, plasma welding and combinations thereof. Which technique to use for a particular purpose can be determined by considering various factors, including by way of example, materials used to make the laminae, architecture of the device, number and position of bonds, etc.

1. Microprojection Welding

Laminae can be bonded to one another at specific sites on the laminae by the novel process of microprojection welding. Microprojection welding comprises patterning lamina with at least one projection, and more typically plural projections, that extends from at least one surface, generally a major planar surface, of the lamina. Selective bonding is accomplished by placing laminae between electrodes and passing a current through the electrodes. The laminae are bonded together selectively at the site or sites of the projection(s). A person of ordinary skill in the art will recognize that a variety of materials suitable for welding can be used to produce the projections, including mild steel, carbon steel, low carbon steel, weldable stainless steel, gold, copper, and mixtures thereof. The welding material (i.e., projections) preferably is made of the same material as the laminae being bonded.

Microprojections suitable for microprojection welding can be produced by both additive and subtractive processes. In one embodiment of the invention, a subtractive process was used to pattern laminae. The subtractive process comprises etching away material from a lamina to produce the microprojections. A person of ordinary skill in the art will recognize that a variety of etching processes can be used, including photochemical and electrochemical etching.

In another embodiment of the invention, microprojections can be patterned on laminae by an additive process. This additive process comprises building up a lamina to produce the microprojections or building up the projections on a lamina prior to lamina patterning. One method of producing the microprojections would involve either etching or depositing projections through a lithographic mask prior to lamina patterning. Lamina patterning should then be conducted with reference to the placement of these projections. For example, if the flapper valve pivot is too close to ring projections, then “flash material” may interfere with the operation of the flapper valve. “Flash material” is extraneous projection weld material or material produced by the welding operation.

Microprojections can have several geometries. For example, individual isolated protrusions can be used. Moreover, continuous lines, rings or any other geometries suitable for the welding requirements of a particular device, can be used to practice microprojection welding of laminae.

In one aspect of the invention, plate electrodes were used to deliver current sufficient to weld the laminae to one another. The laminae that are to be welded together are placed between and in contact with the plate electrodes. Optionally, pressure can be applied to place the laminae in contact with each other or the plate electrodes.

Typical projections of working embodiment had heights of from about 100 μm to about 200 μm, with diameters of about 125 μm or less. If the projections are shorter than 100 μm, electrical shorts may result. The weld nuggets produced by the welding operation had diameters of about 1.5-1.7 mm. It can be important to orient substructures on individual lamina so that weld nuggets produced by the welding process do not overlap, and hence potentially interfere with the operation of, the substructures.

2. Diffusion Soldering

Diffusion soldering is a known method for producing joints. See, for example, D. M. Jacobson and G. Humpston, Diffusion Soldering, Soldering & Surface Mount Technology, No. 10, pp. 27-32 (1992), which is incorporated herein by reference. However, diffusion soldering has not been adapted for use in microlamination processes for bonding laminae one to another for MECS devices.

Diffusion soldering of laminae can be practiced using a number of material combinations, including both base metals and alloys and on surfaces that have been metalized. Two of the more versatile combinations are tin-silver and tin-indium. These two diffusion-soldering systems provide a low-temperature bonding process that results in intermetallic strong joints at the material interface.

Another attractive feature is that the bond produced by diffusion soldering can take considerably higher reheat temperatures than most conventional bonding methods. Because of these characteristics, diffusion soldering is well suited for producing microlaminated devices that must operate at moderate temperatures (i.e., up to approximately 500° C.).

The tin-silver system can work on any surface able to withstand moderate temperatures and capable of receiving a plating layer of the requisite metal. For many devices, steel and stainless steel offer a number of attractive characteristics for fatigue strength, magnetic properties, relatively low thermal conductivity (for stainless steel), and corrosion resistance.

The diffusion soldering method first comprises preparing and plating the surface of each lamina. A typical plating process comprises plating with a low temperature material and a high temperature material. These two materials typically produce an intermetallic material by diffusion soldering.

More specifically, diffusion soldering may involve placing a first strike layer, such as a thin strike layer of nickel (approximately 0.5 μm) on a bare surface that will receive the nickel, such as a metal or alloy surface. This layer promotes adhesion of the other platable metals. Strike layers may not be necessary. Then, a second, generally thicker layer, such as a silver layer 1 μm-10 μm, more typically 2-5 μm thick, is plated over the first layer. Copper may be preferred as a bonding agent between the strike layer or the lamina and the high temperature soldering material because of its ability to readily bond to both nickel and silver. Copper can create a copper-silver intermetallic that is weaker than the surrounding material, and hence be the site of material failure in the device. Finally, a third low-temperature material layer, typically tin, is plated 1 μm-10 μm, preferably 2-5 μm thick over the second layer.

Working embodiments used a stack of laminae having alternating surfaces plated with either high-temperature or high-temperature and low-temperature material, such as silver or silver and tin. The two outside laminae typically have high-temperature material, such as silver, so that the final, bonded stack did not adhere to the alignment jig. If possible, non-bonded internal structures and cavities preferably have the silver layer on their surface. This is to prevent low-temperature material from flowing into features.

The bonding takes place by momentarily raising the stack temperature above the melting point of the low-temperature material (e.g., tin @232° C.) under a compression pressure sufficient to achieve the bond. At higher pressures, lower temperatures likely will be required to achieve adequate bonding. Working embodiments have used compression pressures of approximately 2 MPa to about 5 MPa. A compression pressure below about 2 MPa may not provide sufficient pressure to achieve adequate bonding. Air and other oxidizing atmospheres preferably are excluded at this point to avoid the creation of tin oxides and voids. However, with the surface properly prepared, the bonding process is rapid and complete. One important aspect is to maintain sufficiently low temperatures and pressures so that the lower temperature material does not flow into the features, causing restriction of flow therethrough or therein.

Bond strength and re-heat temperatures can benefit by heating the stack for a longer period of time at the bonding temperature, such as at least up to one hour. This allows tin to further diffuse into the silver and produce stronger intermetallic compounds within the joint itself. Some evidence exists for ultimately producing a silver bond interspersed with intermetallic tin/silver particles yielding a high strength, moderate temperature joint. Indium can be used in place of tin to yield an even lower temperature (melting point of indium is 157° C.) bonding process.

3. Miscellaneous Bonding Methods

Polyimide sheet adhesives can be used to bond laminae together. Polyimide is a commercially available, high-strength, high-temperature polymer. For example, Dupont manufactures a polyimide sheet adhesive, Kapton KJ. Kapton KJ retains adhesive properties and can bond surfaces together when heated and compressed. Polyimide sheets produce moderate strength bonds that also provide good sealing capability.

E. Component Dissociation by Eliminating Fixture Bridges

Component dissociation is accomplished by eliminating fixture bridges. It will be recognized that there are a variety of ways to eliminate fixture bridges, including vaporizing the fixture bridge by heating it to a sufficient temperature, chemically eliminating, such as by dissolving, the fixture bridge, and laser ablation of the fixture bridge. Combinations of these methods also can be used.

One method for vaporizing the fixture bridges comprises capacitive discharge dissociation. Capacitive discharge dissociation comprises applying a current through the fixture bridge sufficient to vaporize the fixture bridge. There are a variety of ways to apply current through a fixture bridge. Working embodiments of the method have placed a first electrode in contact with the structure and a second electrode in contact with the substructure to be dissociated. Current is passed between the electrodes.

In one embodiment of the invention, a DC power source was used to charge a capacitor. The capacitor was discharged to pass current through the electrodes. The temperature, the amount of current, and the power necessary to eliminate the fixture bridge often varies with the particular properties of the fixture bridge, including the material the fixture bridge is made of, its cross-sectional area, and its length.

In another embodiment of the invention, fixture bridges are eliminated by thermochemical dissociation. Thermochemical dissociation has the potential advantage of reducing debris that may be produced during fixture bridge elimination. Thermochemical dissociation comprises selectively heating the fixture bridges, in combination with chemical elimination. Selective heating of the bridge can be accomplished by applying current to the fixture bridge, heating with a laser and/or focusing a laser on the bridge. One way to apply current through the fixture bridge comprises placing electrodes at or near the ends of the fixture bridge and passing a current between the electrodes. In another embodiment of the invention, heating elements, or some other method for delivering thermal energy, can be used to selectively heat the fixture bridges.

Chemical elimination also comprises applying a sufficient amount of a chemical to eliminate the fixture bridges. The fixture bridges also optionally can be selectively heated to a temperature sufficient to help chemically eliminate them either prior to, subsequent to, or simultaneously with application of the chemical. There are a variety of chemicals that can be used to eliminate the fixture bridges, such as acids, particularly mineral acids, bases, oxidizing agents, and mixtures thereof. The concentration, pH, and temperature sufficient to selectively chemically eliminate the fixture bridges varies with the particular properties of the fixture bridge, including the material the fixture bridge is made of, the cross-sectional area, and the length. Preferably, an acid having a pH of less than about 3 and at a temperature above freezing temperature is applied to the lamina. Preferably, the fixture bridges are heated to temperatures from about 200° C. to about 300° C. If the laminae are made of a copper alloy, cupric chloride or ferric chloride can be used to chemically eliminate the bridge. If the laminae are made of steel, a mixture, such as a 1:1 volume mixture of HCl:HNO₃, can be used to eliminate the fixture bridge.

In another embodiment of the invention, fixture bridges are eliminated by laser ablation. In this embodiment, line-of-sight access to the fixture bridges from the exterior of the device is desired. The laser beam should be able to be focused onto the fixture bridge, which may require line-of sight access. UV lasers are particularly useful as they ablate metals as well as polymers and ceramics with little heat affect and very sharply distinguished features. Laser ablation allows the fabrication of preassembled features in materials other than metals, such as polymer and ceramics. An Nd:YAG laser operating in the fourth harmonic (266 nm wavelength) would be an example of a UV laser with sufficient power to perform this operation.

Fixture bridges can be eliminated either prior to, subsequent to, or simultaneously with bonding of the plural laminae. In one embodiment of the invention, the fixture bridges are eliminated prior to the bonding of the plural laminae one to another.

The method of this invention can be used to fabricate freeform geometries and microfeatures within a device. Microfeatures are of the size of from about 1 μm to about 100 μm. The methods of the invention can be used to produce micro-scale and meso-scale devices. Micro-scale devices are of the size of from about 1 μm to about 1 mm, preferably from about 1 μm to about 500 μm, and even more preferably from about 1 μm to about 100 μm. Meso-scale devices are of the size of from about 1 mm to about 10 cm, preferably from about 1 mm to about 5 cm, and even more preferably from about 1 mm to about 1 cm. Arrays of preassembled, meso-scale devices can be fabricated with overall sizes of up to about 12.5 centimeters by about 12.5 centimeters.

F. Microlamination Using Polymeric Materials

Many MECS devices require the integration of various types of membranes within a microlaminated stack. Examples include integrating Pd membranes for hydrogen separation within microchannel fuel processing systems, integrating contactor membranes in microchannel absorbers for use in heat pumps, integrating separation membranes into microchannel dialyzers for portable kidney dialysis, and integrating elastomeric membranes into highly-branched networks of microreactors for molecular manufacturing (e.g. dendrimer synthesis). Each case requires integrating heterogeneous materials into a laminated stack. Problems with membrane integration within embedded microchannel systems can include:

1. Membrane materials are typically quite expensive and so it is desirable to minimize the amount of membrane material used. This typically sets the requirement for using a second, less expensive packaging material that needs to be integrated with the membrane material.

2. Membrane materials often have specific nano- or micromorphologies, which dictate the mass transfer of the membrane. These morphologies are many times sensitive to heat and pressure and other processing conditions. Therefore, these materials cannot be conveniently patterned into geometries compatible with microchannel designs and a mechanism is needed to incorporate the raw material form within the microlaminated stack.

3. Many times the techniques used to bond a single material are complicated when bonding different materials. An example might be the ultrasonic welding or thermal bonding of two polymers with significantly different glass transition temperatures where the form of is compromised before the other is ready for welding. Solvent welding may requires using different solvents for different materials. Plasma oxidation produces excellent welds between polydimethylsiloxane and polyethylene or polystyrene, but may not be useful for other combinations of materials.

4. Membranes often are of a thickness, or are made out of a material, that results in poor stiffness. But, microchannels with interspersing membranes must be produced that do not have significant fin warpage and channel non-uniformities. Channel non-uniformities can lead to flow maldistribution, which can negatively impact the effectiveness of heat exchangers and microreactors.

5. The low modulus of some membranes requires that the layers be thick (on the order of one mm) in order to maintain dimensions. Therefore, in order to reduce the fluid volume of certain microchemical nanofactories while meeting processing and operating requirements, it is desirable to integrate elastomeric capabilities of polymeric materials, such as PDMS, with a stiff material, such as polycarbonate.

While some membranes are excellent candidates as valve membranes or other purposes, they are not a good for packaging. Separation membranes often are highly gas permeable, which can cause evaporation in microchannels and lead to vapor-lock. And, some membranes are not suitable as substrates for thin film deposition of heaters and thermocouples.

G. Membrane Integration Techniques

1. PDMS Integration

One method for bonding PDMS to another surface involves plasma oxidation of the PDMS surface followed by conformality to the faying surface. Plasma oxidation introduces silanol (Si—OH) groups on the surface of PDMS and the condensation reaction of these groups with appropriate groups (such as OH, COOH, ketones) on the surface of another material or PDMS forms a strong bond between the two surfaces and can immobilize the grafted layer. The oxidized PDMS surface ma become inactive if not stabilized in aqueous solution within minutes after plasma oxidation. PDMS also is compatible with only a handful of materials including glass, silicon, silicon oxide, silicon nitride, polyethylene and polystyrene. Silicon and glass surfaces are expensive relative to polymeric surfaces. Polystyrene and polyethylene, which can be grafted to PDMS, are not suitable for thin film deposition. Ticona Topas (COC), Zeonor 1600 and GE HPS1/HPS2 are examples of structural polymers having excellent optical clarity, high modulus, high glass transition temperature (>150° C.) and low gas permeability suitable for thin film deposition. Therefore, integration of PDMS with cheap, structural polymers can be used to make microchemical nanofactories.

One specific approach for integrate PDMS membranes is to formulate copolymers with protected functionality under atmospheric conditions which will polymerize under selective exposure to UV light. Two specific procedures are as follows. Hydride functional (Si—H) siloxanes have been incorporated into silanol elastomer formulations to produce foamed structures. Based on this, a novel and plausible approach to impart bonding character on PDMS, without plasma oxidation, is to incorporate a small amount (less than 1%) of silanol functional siloxane (or polysilsesquioxane) into the vinyl-addition siloxane formulation and selectively cure the blend. Also, a methacrylate or acrylate functional siloxane copolymer (which cures on exposure to UV) can be incorporated into the vinyl-addition siloxane such that selective curing of the blend can be used to achieve bonding of surfaces. Oxygen inhibits the polymerization of methacrylate, so the methacrylate functionality may be protected in the presence of oxygen and unprotected to obtain a reasonable cure when blanketed with nitrogen or argon during UV exposure.

2. Physical Constraint of Membranes

Another approach to membrane integration is to physically constrain membrane layers between stiff layers of molded polymers (e.g. Ticona Topas COC, Zeonor 1600 and GE HPS1/HPS2). Because of the stiffness of these materials, each makes an excellent candidate for ultrasonic welding. In addition, as thermoplastics, each has the ability to be thermally bonded (PDMS has a degradation temperature well above the Tg of these materials) and solvent welded.

3. Ultrasonic Welding

Ultrasonic welding enables integration of the microinjection, microreaction, microseparation, detection and microextraction subsystems within a microreactor design for synthesizing chemical compounds, such as dendrimers. Dead space within the microsystem is minimized by using stiff polymer films in place of thick PDMS substrates. These same concepts of physical constraint can be extended to many different heterogenous microlaminated platforms.

Methods employed in the fabrication of test articles include micro hot embossing, laser micromachining and spin casting. A PDMS valve membrane may be sandwiched between two polycarbonate layers using ultrasonic welding. In order to accomplish this, angled channels are CNC machined into the stainless steel substrate after Ni electroforming and resist stripping. These form raised ridges during embossing that act as energy directors for ultrasonic welding.

The elastomeric valve membrane layer is formed by spin casting PDMS monomer onto a wafer with raised photoresist features that form the valve chambers, curing, and then laser machining openings for protrusion of the ultrasonic energy directors. This PDMS membrane layer could be replaced by any type of off-the-shelf membrane

### IV. Unit Operations

The present invention is particularly directed to integrated nanofactories, modular nanofactories, or systems comprising combinations of integrated and modular nanofactories. Nanofactories include various structures useful for providing reactants, making compounds using reactants, separating and/or purifying made compounds, and/or analyzing compounds made using the nanofactory(ies). Embodiments of various individual components that also may have utility when used alone are discussed first, followed by descriptions of various embodiments of modular and integrated devices.

Disclosed embodiments of nanofactories can be used for continuous processes. For these situations, it may be beneficial to balance the output of a first unit operation with the ability of a downstream unit operation to process the fluid output from the first unit operation. Solely by way of example, a single mixer may be able to process more fluid than certain downstream devices, such as separators. Thus, in this example, the nanofactory may need to include more separators than mixers in order to continually process the output from a first unit operation, such as the mixer.

A. Mixers|_{[bkp1]}

Most chemical syntheses involve mixing two or more reagents together to facilitate reaction and formation of desired products. As a result, micromixers are a first example of a unitary device that can be incorporated into an integrated factory.

Microreaction technology offers several new opportunities to suppress the competing side reactions and maximize the purity of products made. With respect to dendrimer synthesis, the conversion rate of the alkylation/amidation reaction sequence may be increased by enhancing effective collision between reactants to create a microfluid (mixing of reactants at the molecular level) rather than a macrofluid (aggregates of separate reactants).

Mixing typically involves integration of one or more fluids into one phase and molecular diffusion is usually the final step in all mixing processes. A simple estimation shows that it will take five seconds to mix two contacting 100 μm-thick aqueous laminar layers containing small molecules and would only take 50 milliseconds if the layers were 10 μm. The essence of mixing thus relies on the concept of volume division. One common approach to achieve volume division is through creation of a turbulent flow. The fluid is subdivided into thinner and thinner layers by eddies. A large number of mixing apparatuses use this approach. It is difficult to achieve uniform mixing at the micrometer scale in a short time using traditional mixing apparatuses, such as paddles or propellers in a reaction tank. This is evident in many cases where the experimental measured kinetics depends strongly on the stirring conditions even in a laboratory scale reactor. Micromixers offer features which cannot be easily achieved by macroscopic devices, such as ultrafast mixing on microscale. For example, Bökenkamp et al. fabricated a micromixer as a quench-flow reactor to study fast reactions (millisecond time resolution). D. Bökenkamp, A. Desai, X. Yang, Y.-C. Tai, E. M. Marzluff, S. L. Mayo., Anal. Chem. 70, 232-236, 1998.

A variety of micromixers have been reported in the literature including static and dynamic mixers. See, for example, Lowe, H., W. Ehrfeld, V. Hessel, T. Richter and J. Shiewe. 2000. “Micromixing Technology,” Proceedings of IMRET 4, AIChE Spring National Meeting, Atlanta, Ga., pp. 31-47; J. B. Knight, A. Vishwanath, J. P. Brody, R. H. Austin, Phys. Rev. Letts. 80(17), 3863-3866, 1998; N. Schwesinger, T. Frank, and H. Wurmus, J. Micromech. Microeng. 99-102, 1996; A. D. Stroock, S. K. W. Dertinger, A. Ajdari, I. Mezić, H. A. Stone, G. M. Whitesides, Science 295, 647-651, 2002; H. H. Bau, J. Zhong, M. Yi, Sensor Actuators B 79, 207-215, 2001; and M. Oddy, J. G. Santiago, J. C. Mikkelsen, Anal. Chem. 73, 5822-5832, 2001. The envisioned applications involve miscible fluids with high diffusivity in one another and are therefore amenable to static diffusional mixing. Sub-second mixing times exist in the literature for static, diffusional mixers. See, Ehrfeld, W., K. Golbig, V. Hessel, H. Lowe and T. Richter. 1999. “Characterization of mixing in micromixers by a test reaction: Single mixing units and mixer arrays,” Ind. Eng. Chem. Res. 38(3): 1075; and van den Berg, A. 1998. “Miniaturized systems for chemical and biochemical analysis,” CIT 70(9): 1076.

1. Interdigital Micromixers

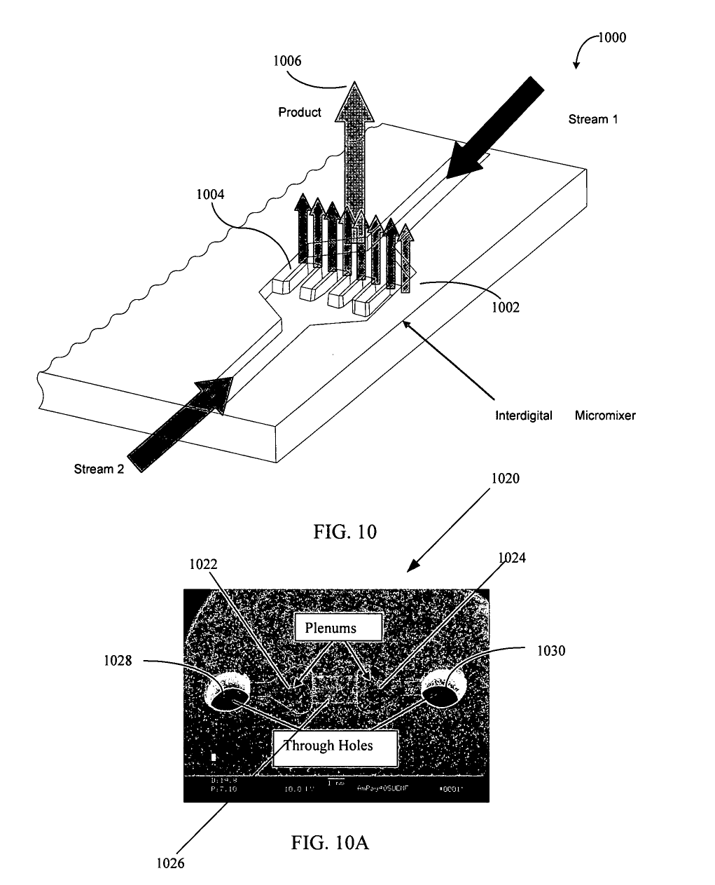

FIG. 10 illustrates one embodiment of an interdigital micromixer 1000. Fluids A and B to be mixed are introduced into the mixing element 1002 as two counter-flows. Mixing element 1002 includes plural interdigital channels 1004, each of which typically has a channel width of from about 20 μm to about 50 μm. Fluids A and B split into many interpenetrated substreams. Substreams 1006 exit the interdigital channels 1004 perpendicular to the direction of the feed flows A and B, initially with a multilayered structure. Mixer 1000 can be manufactured using polymeric microlamination architecture using replica molding/polymer embossing and various bonding strategies. Spacing between digits on the order of 20 μm likely can be achieved providing mixing times on the order of a few hundred milliseconds depending upon flow rates. Such mixers have been used by the present inventors to generate a cadmium sulfide (CdS) nanoparticle solution using a PDMS interdigital micromixer. Stable monodispersed CdS nanoparticle suspensions were produced even without adding stabilizers.

FIG. 10A is a photomicrograph of an interdigital mixer 1020. Mixer 1020 includes plenums 1022 and 1024 operatively associated with the interdigital mixing section 1026. Plural fluid feed apertures 1028 and 1030 are operatively associated with each of the plenums 1022 and 1024. Plenums 1022 and 1024 facilitate distribution of fluid from the feed apertures 1028 and 1030 to the entire mixing section 1026. Without such plenums 1022 and 1024, fluid entering the mixing section 1026 impinges only a portion of the mixing section, and hence mixing and/or throughput is not as efficient as is realized by using plenums 1022 and 1024.

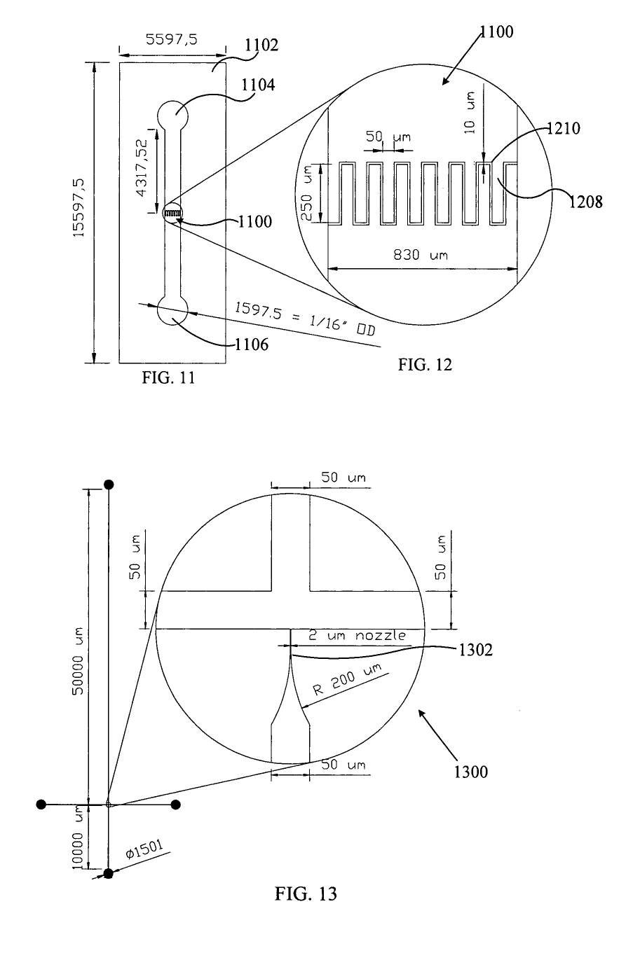

FIGS. 11 and 12 also illustrate an interdigital mixer 1100. FIG. 11 illustrates an interdigital mixer 1100 as part of a lamina or laminae 1102 that defines ports 1104 and 1106 for fluid flow. Exploded view 12 provides dimensions for one embodiment of such an interdigital mixer 1100. It will be understood that these dimensions are exemplary only. For the illustrated mixer 1100, fluids flowing to the mixer enter channels 1208 having a width of approximately 50 μm and a length of about 250 μm. The overall width of the mixer 1100 is about 830 μm, and the thickness of the wall 1210 defining the mixer is about 10 μm. As with the embodiment of FIG. 10, counter flowing fluids impinging the mixer 1100 create a combined fluid stream (not shown) that thereafter flows perpendicular to the flow direction of the two initial streams.

2. Nozzle Micromixers

FIG. 13 illustrates an embodiment of a nozzle mixer 1300. The illustrated embodiment of micromixer 1300 has a nozzle 1302. While the dimensions of such nozzle mixers may vary, the illustrated embodiment typically has a nozzle of from about 2 to about 10 μm with relatively high aspect ratios typically greater than about 30:1. These aspect ratios are currently beyond the limit of planing and micro hot embossing techniques, and hence UV photolithography in thick resist (SU-8) are used to make embodiments such as the micromixer 1300. To package the device, a thin layer of silica will be sputter coated onto the surface of SU8 and a thin layer of an elastomer, such as polydimethylsiloxane, will act as a seal between the SU8 structure and a polycarbonate substrate. A working embodiment of mixer 1300 will have layers in the following order: glass or polycarbonate, PDMS, silica, SU8, and stainless steel or silicon wafer substrate.

To perform photolithography, a photomask had to be designed, and was sent to Photo-Sciences for production. By using an optical microscope, the dimensions on the photomask all met the requirements of the illustrated design to allow production of a nozzle mixer 1300 that has a 1.1 μm nozzle opening and 19.4 μm channel.

3. Microjet Micromixers

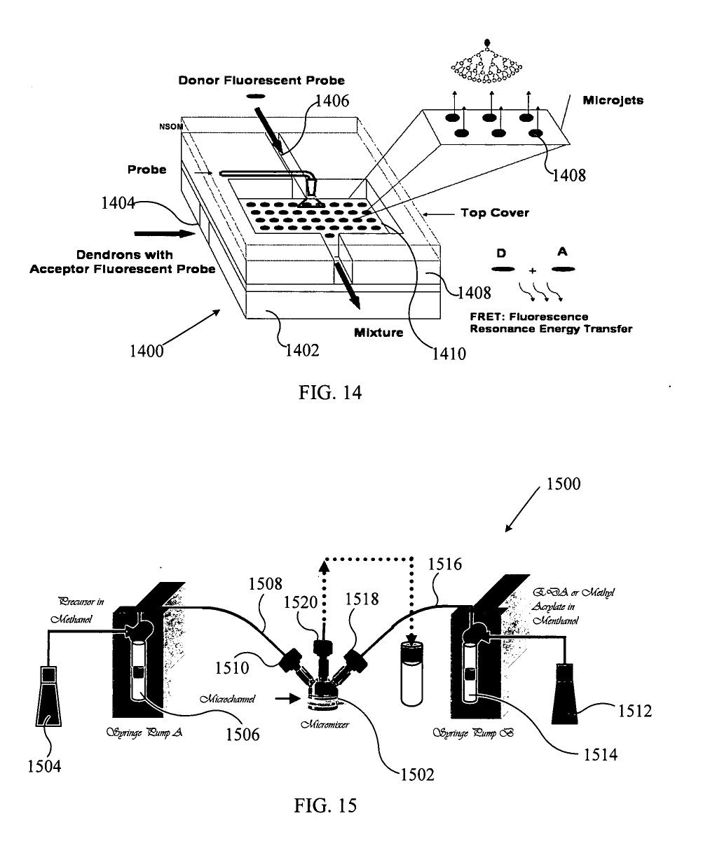

One embodiment of a microjet array mixer is illustrated in FIG. 14. Mixer 1400 can be made using a microlamination architecture. The illustrated embodiment of mixer 1400 includes a first layer 1402 that defines a first microchannel 1404 for introduction of fluid flow to the mixer 1400. A second fluid may be introduced by a second microchannel 1406 produced either in the same layer 1402 or a separate layer 1408 as with the illustrated embodiment. A first fluid flowing in microchannel 1404 is then introduced into a mixing area 1410 that includes plural mixing ports or microjets 1412 (see the exploded view).

Additional examples of planar microjet mixers have been made. For example, membranes with straight-through pores down to 5 μm have been laser micromachined in 75 μm thick Kapton KJ and micromolded in 40 μm thick. Even at 100-μm spacing between pores at a mass flux of 0.5 g/min/cm², pressure drop across the membrane has been measured to be only a few torr.

4. Commercially Available Micromixers

Certain embodiments of micromixers are commercially available. One such mixer 1502 is illustrated in FIG. 15. Micromixer 1502 is not a chip-based mixer, nor is it integrated with other components that might be compiled to define a chip-based microfactory as contemplated herein. Interdigital micromixer 1502 (SSIMM from Institut für Mikrotechnik Mainz, Germany) consists of interdigital microchannels (not shown) embossed in the center of the substrate made of thermally grown silicon dioxide. The mixing element is hosted within a stainless steel container. Each microchannel has a dimension of 30 μm in width and 100 μm in length.

Microreactors improve mixing and heat transfer due to short diffusion pathways, and large interfacial areas per unit volume (10,000˜50,000 m²/m³), respectively. In contrast, conventional reactors only have the ratio of the area versus volume with 100 m²/m³. These two features improve yield and selectivity, specifically for mass-transport controlled reactions, highly exothermic or endothermic reactions and reactions with inherently unstable intermediates. In addition, another attractive advantage is that laboratory scale reactions typically conducted with such micromixers can be easily increased for large production scale by operating plural such microreactors in parallel.

Microreactors suppress the competing side reactions and maximize the purity of products by uniform and precise temperature control, low moisture permeability, and increasing the conversion rate of the alkylation/amidation reaction sequence by effective reactant mixing. Mixing typically relies on volume division and follows by integration of one or more fluids into one phase.

EDA-core PAMAM synthesis has been accomplished using the continuous flow microreactor 1502 of FIG. 15. Microreactor 1502 was used in a system 1500 for the synthesis of the PAMAM dendrimers. System 1500 includes a container 1504 for delivering a precursor in a suitable solvent, such as methanol, to mixer 1502. Syringe pump 1506 pumped the precursor through fluid line 1508 to an inlet port 1510 of micromixer 1502. A second container 1512 contained a reactant, such as EDA or methyl acrylate in a suitable solvent, such as methanol. A second syringe pump 1514 delivered the reactant to the micromixer 1502 via fluid line 1516 through fluid inlet port 1518 of micromixer 1502. Product was then delivered through outlet port 1520 of micromixer 1502.

5. T-Mixers

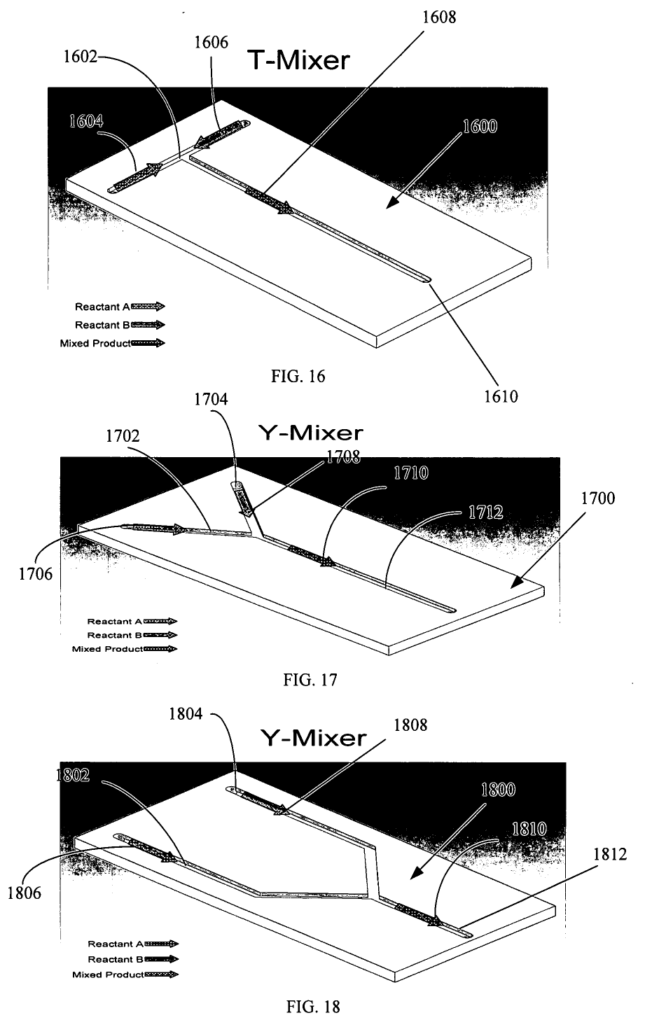

FIG. 16 illustrates one embodiment of a T-mixer 1600. T-mixer 1600 has a first microchannel 1602 for receiving fluid flows of a first reactant A, designated as 1604, and a second reactant B, designated as 1606. Reactants A and B are then mixed as the two fluid streams impinge to form a mixed product flow 1608 in microchannel 1610.

6. Y-Mixers

FIGS. 17 and 18 illustrate two exemplary embodiments of Y-mixers. With reference to FIG. 17, a first embodiment 1700 of a Y-mixer has a first microchannel 1702 and a second microchannel 1704. Microchannels 1702 and 1704 are positioned at an angle relative to one another, such as an angle greater than 0°, such as with the T-mixer to an angle less than 90°. Microchannel 1702 receives a first fluid flow of a first reactant A, designated as 1706. Microchannel 1704 receives a second reactant B, designated as 1708. Reactants A and B are then mixed as the two fluid streams impinge to form a mixed product flow 1710 in microchannel 1712.

FIG. 18 illustrates a second embodiment of a Y-mixer 1800. Y-mixer 1800 has a first microchannel 1802 and a second microchannel 1804. In contrast to the embodiment of FIG. 17, Y-mixer 1800 has microchannels 1802 and 1804 initially providing substantially parallel fluid flow of a first reactant A, designated as flow 1806, and a second reactant B, designated as flow 1808. However, fluid flows 1806 and 1808 then enter a second portion of the microchannels 1802 and 180 that are positioned at an angle r greater than 0° and less than 90°. Reactants A and B are then mixed as the two fluid streams 1806 and 1808 impinge to form a mixed product flow 1810 in microchannel 1812.

7. Branched-Mixers

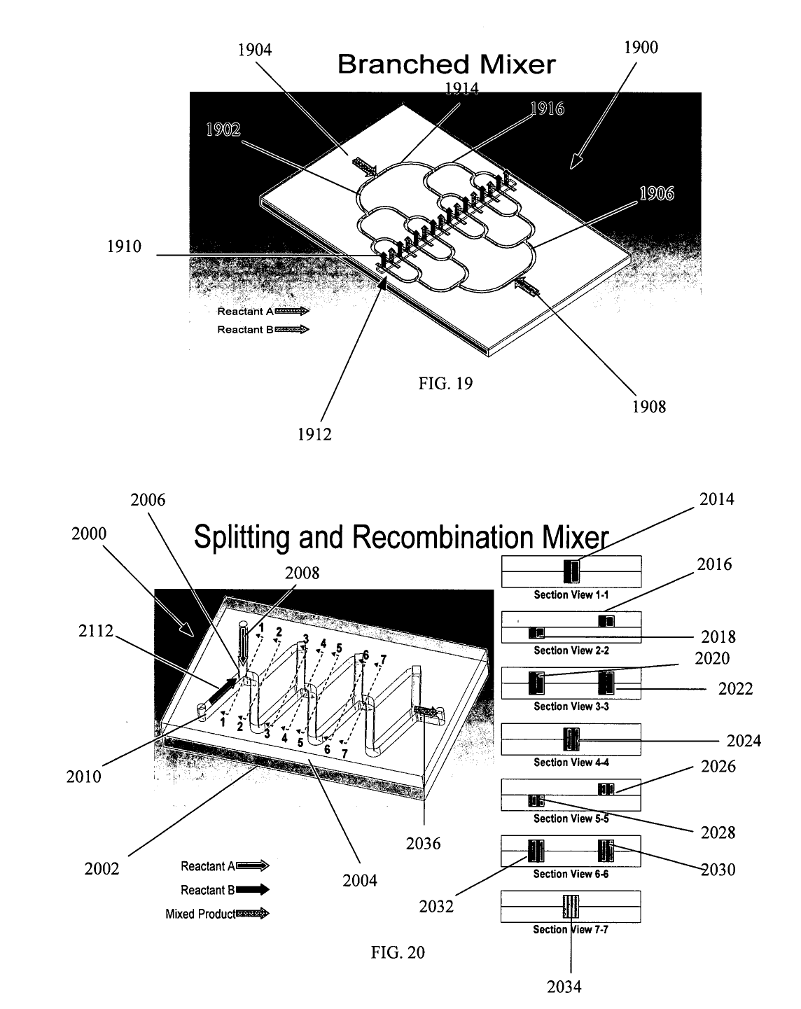

FIG. 19 illustrates one embodiment of a branched mixer 1900. Mixer 1900 includes a first microchannel 1902 for receiving a flow 1904 of a first reactant A. Mixer 1900 also includes a second microchannel 1906 for receiving flow 1908 of a second reactant B. Thereafter, product flow 1910 is formed in mixing section 1912, in much the same manner as with the interdigital mixers of FIGS. 10-12. As illustrated, microchannels 1902 and 1904 branch to form additional microchannel segments, such as segments 1914 and 1916, prior to reaching the mixing section 1912.

8. Splitting and Recombination Mixer

FIG. 20 illustrates one embodiment 2000 of a splitting and recombination mixer. The illustrated embodiment has a first lamina 2002 and a second lamina 2004 that collectively define mixer 2000. Mixer 2000 includes a first microchannel 2006 for receiving a flow 2008 of a first reactant A. Mixer 2000 also includes a second microchannel 2010 for receiving a flow 2012 of a second reactant B. A mixed product stream is formed, in a single microchannel 2014, as indicated by first insert cross section view 1-1. The mixed product stream is then split into two fluid streams in two microchannels 2016, 2018, as indicated by section view 2-2, each stream flowing in individual lamina 2002, 2004. Section view 3-3 shows that the two fluid flows in microchannels 2016, 2018, then flow into microchannels 2020, 2022 defined by laminae 2002, 2004 collectively. Split fluid streams flowing in microchannels 2020 and 2022 then recombine in a single microchannel 2024, as indicated by section view 4-4, to again form a mixed product stream. The mixed product stream is then split into two fluid streams in two microchannels 2026, 2028, as indicated by section view 5-5, each stream flowing in individual lamina 2002, 2004. Section view 6-6 shows that the two fluid flows in microchannels 2026, 2028, then flow into microchannels 2030, 2032 defined by laminae 2002, 2004 collectively. Split fluid streams flowing in microchannels 2030 and 2032 then recombine in a single microchannel 2034, as indicated by section view 7-7, to again form a mixed product stream 2036. A person of ordinary skill in the art will appreciate that the number of times a product stream is split and then recombined is variable, and the present embodiments are not limited to those number of splits and recombinations illustrated in FIG. 20.

9. Collision Mixer

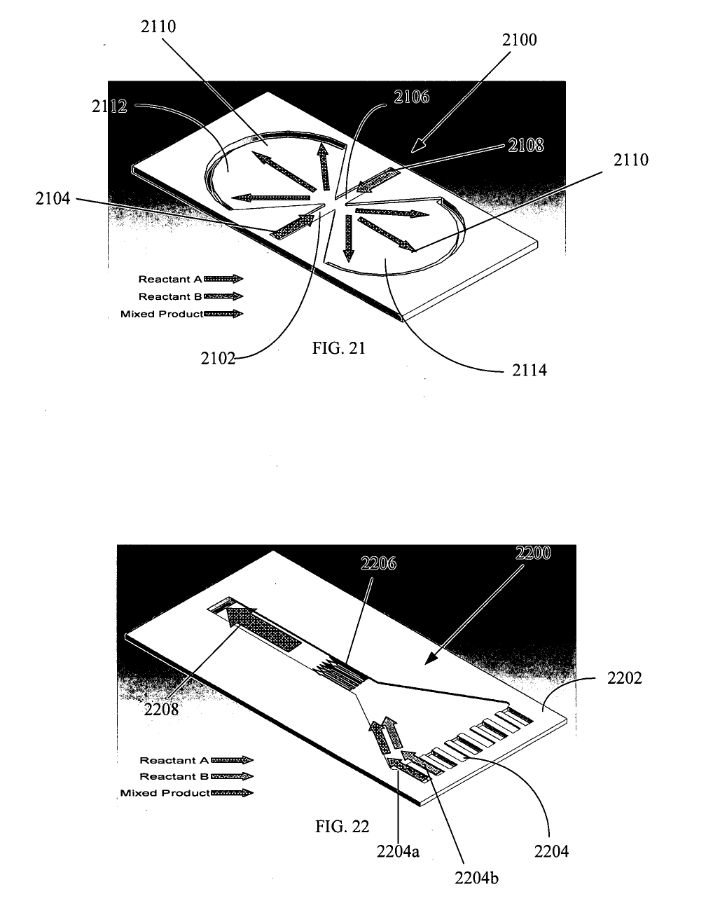

FIG. 21 illustrates one embodiment of a collision mixer 2100. Mixer 2100 has a first microchannel 2102 for receiving fluid stream of a first reactant A, designated as 2104. Mixer 2100 includes a second microchannel 2106 for receiving fluid stream of a second reactant B, designated as 2108. Reactants A and B are then mixed as the two fluid streams impinge to form a mixed product flow 2110. Mixed product flow 2110 flows radially outwardly from microchannels 2102 and 2106 into product receiving chambers 2112 and 2114.

10. Superfocusing Mixer

FIG. 22 illustrates one embodiment of a superfocusing mixer 2200. First end 2202 of mixer 2200 has plural reactants inlet 2204. For example, a first reactant A is provided to the superfocusing mixer 2200 via a reactant microchannel 2204a. A second reactant B is provided to the superfocusing mixer 2200 via a reactant microchannel 2204b. Fluid flow in a microchannel typically is lamellar flow, as indicated in FIG. 22 as section 2206. However, as two fluids flow adjacent each other, interdiffusion of the two fluids may occur, thereby forming a mixed product flow 2208.

11. Serpentine Mixer

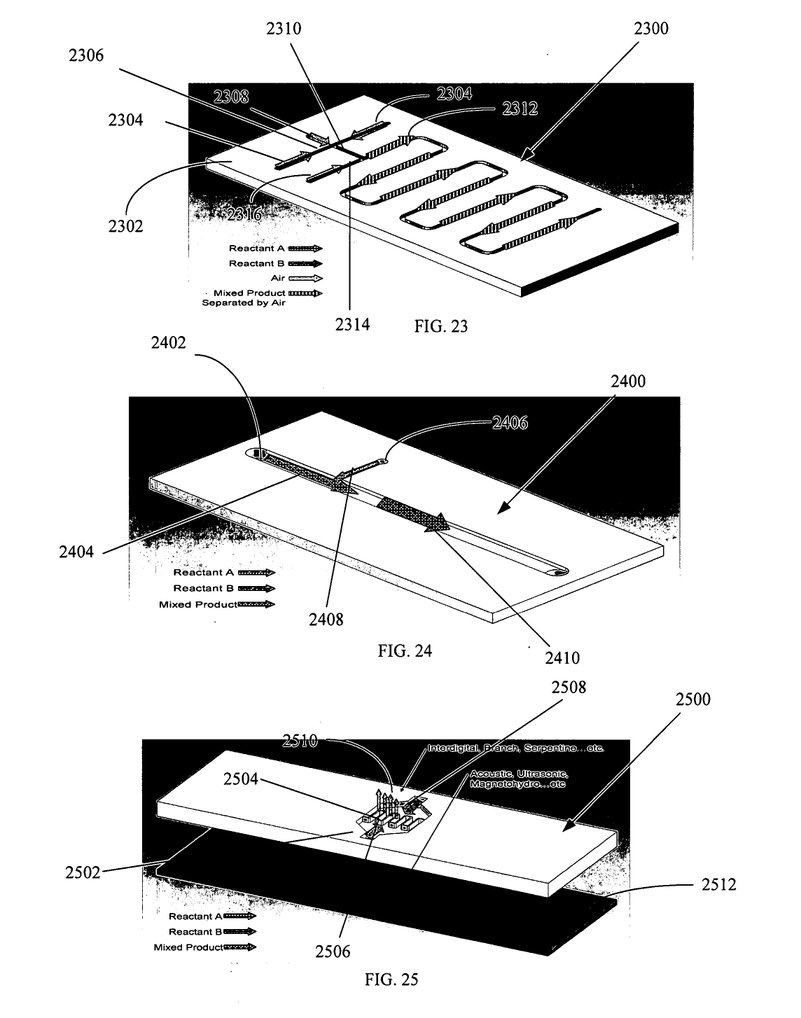

FIG. 23 illustrates one embodiment of a serpentine mixer 2300. The illustrated embodiment also is configured to provide segmented fluid flow. A first end 2302 of mixer 2300 has plural reactant inlets. For example, a fluid flow 2304 of a first reactant A is provided to the serpentine mixer 2300 via a reactant microchannel 2306. A fluid flow 2308 of a second reactant B is provided to the serpentine mixer 2300 via a reactant microchannel 2310. As with the T-mixer, fluid flows 2304 and 2308 impinge to form a mixed product flow 2312. A third microchannel 2314 is provided. In the illustrated embodiment, a third fluid flow 2316, such as air, is introduced into the mixer 2300. Fluid flow 2316 can be provided either continuously, or as a pulse. Fluid flow 2316 can be used to provide segmented flow of mixed product fluid 2312.

12. Venturi Mixer

FIG. 24 illustrates one embodiment of a venturi mixer 2400. Venturi mixer 2400 has a first microchannel 2402 for receiving a fluid flow 2406 of a first reactant A. Mixer 2400 includes a second microchannel 2406, typically having smaller dimensions that microchannel 2402, for introducing a second fluid flow 2408 of a reactant B. A venturi is a restricted fluid inlet that produces a drop in pressure, causing fluid to be drawn out of the microchannel. Reactants A and B are then mixed as the two fluid streams impinge to form a mixed product flow 2410.

B. Passive and Active Mixers

Mixing operations can be passive, that is mixing operations that are not facilitated by other processes, such as agitation, or by application of energy from an energy source. Conversely, disclosed embodiments of mixers may be used in an active mixing process. Thus, each of the mixer embodiments disclosed herein, and mixers and mixing methodologies not affirmatively disclosed but within the scope of the present invention, can be passive or can be an active mixing process.

One embodiment of an active mixing system 2500 is illustrated schematically in FIG. 25. System 2500 includes a mixing section 2502. The illustrated embodiment includes a interdigital mixer 2504. A first reactant 2506 is mixed with a second reactant 2508 to produce a mixed product 2510. System 2500 also includes a lamina 2512 positioned adjacent mixing section 2502. Lamina 2512 provides a material that produces, or receives and radiates, energy to the mixing section 2502. For example, and without limitation, lamina 2512 may be an acoustic energy layer, an ultrasonic energy layer, a magnetohydro layer, etc.

C. Valves

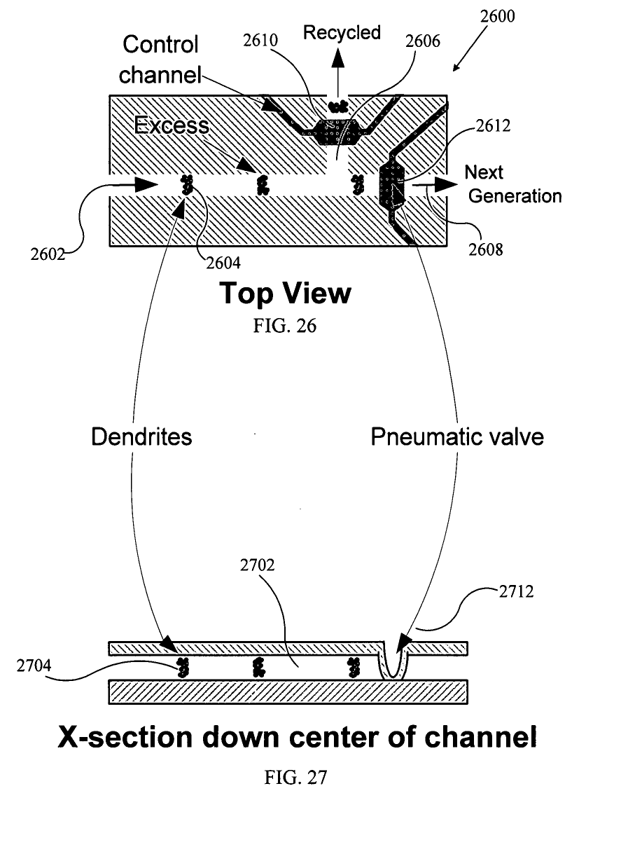

The need for valves in an integrated system soon becomes apparent. Some actuatable microvalves are known. For example, a pneumatically actuated valve conceived by Thorsen et al. is illustrated in FIGS. 26 and 27. FIG. 26 is a plan view illustrating a system 2600 for production of dendrites. System 2600 includes a first channel 2602 through which dendrites 2604 flow. Recyclable material needs to be separated from material used to make the next dendrite generation. The recyclable material is guided down channel 2606 and next generation material is guided down channel 2608. System 2600 includes two pneumatically actuatable valves 2610 and 2612. A person of ordinary skill in the art will appreciate that similar valves can be integrated into the systems of the present invention, and further that the valves can be generally fluidly actuatable, need not solely be pneumatically actuatable, and can be, for example, hydraulically actuatable. By appropriate actuation of valves 2610 and 2612, recyclable material can be guided down channel 2606 and next generation material can be guided down channel 2608, as desired.

FIG. 27 is a cross sectional view of the system 2600 illustrated in FIG. 26. FIG. 27 illustrates deflection of the valve 2712 into and blocking channel 2702.

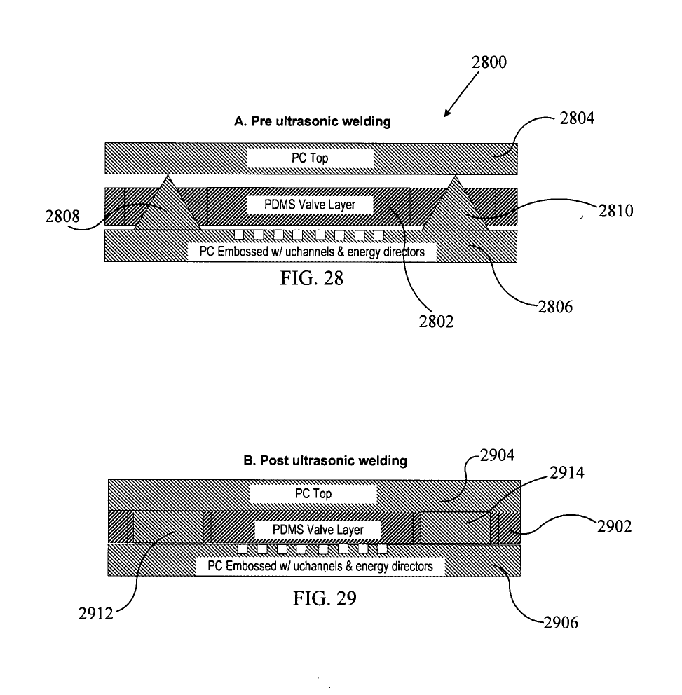

FIGS. 28 and 29 illustrate an ultrasonic method for making a fluidly actuatable valve in a system 2800. FIG. 28 illustrates packaging an elastomeric valve membrane 2802, such as a polydimethylsiloxane (PDMS) elastomer, between two polycarbonate layers 2804, 2806 using ultrasonic welding. In order to accomplish this, angled channels are CNC machined into a stainless steel substrate after Ni electroforming and resist stripping. These yield raised ridges 2808, 2810 during embossing that act as energy directors for ultrasonic welding.

The elastomer valve membrane layer 2802 was patterned by spin casting PDMS monomer onto a wafer with raised photoresist features that produce the valve chambers, curing, and then laser machining openings for protrusion of the ultrasonic energy directors. FIGS. 28 and 29 are schematic cross sections prior to and subsequent to ultrasonically welding, respectively, with energy directors 2808, 2810 protruding above elastomeric valve layer 2302. FIG. 29 diagrams the result of ultrasonic welding with the energy directors 2908, 2910, melted down and bonding the top and bottom polycarbonate layers 2904, 2906 and compressing the PDMS layer 2902 and sealing the microchannels 2912 and 2914.

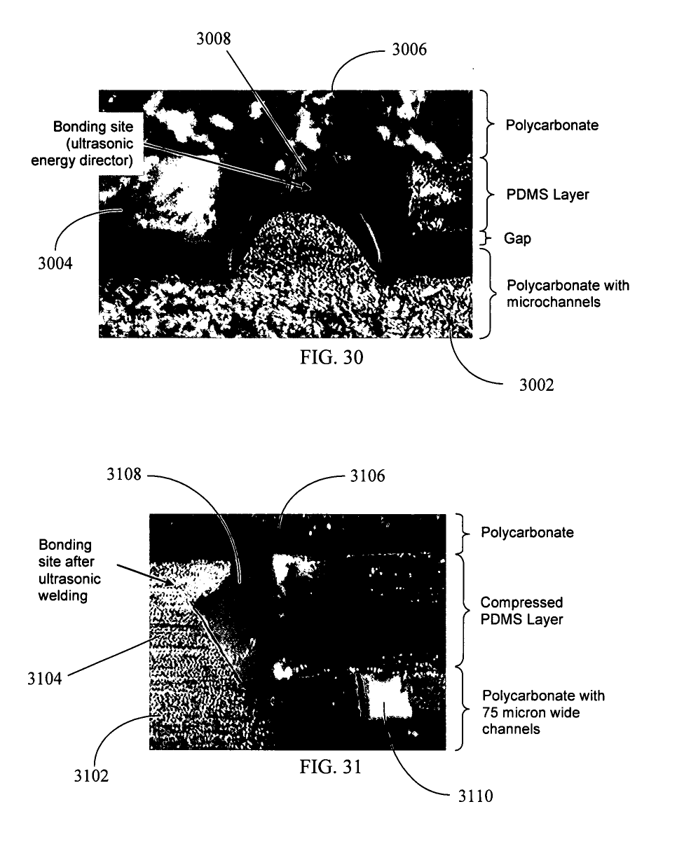

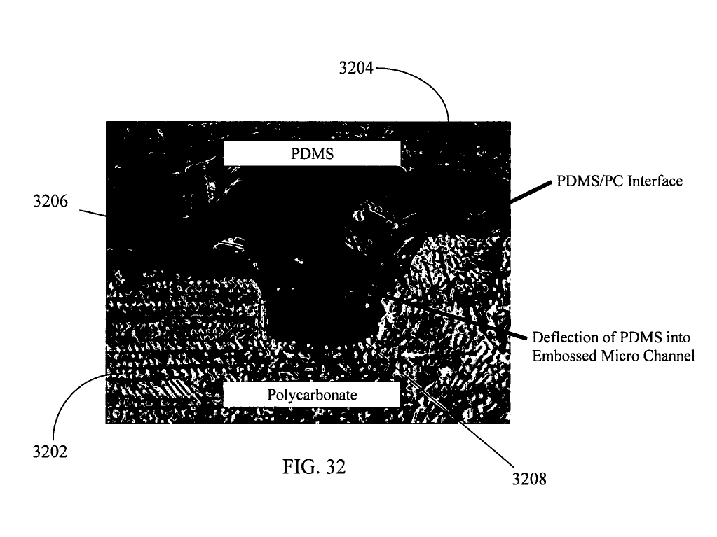

FIGS. 30-32 are photomicrographs of working embodiments of such systems. FIG. 30 illustrates a system prior to welding comprising a first polycarbonate lamina 3002, a second polydimethylsiloxane (PDMS) layer 3004, and a third polycarbonate layer 3006. FIG. 30 also illustrates using an energy director, such as an ultrasonic energy director, 3008. FIG. 31 illustrates the system subsequent to welding. FIG. 31 illustrates a system comprising a first polycarbonate lamina 3102, a second polydimethylsiloxane (PDMS) layer 3104, and a third polycarbonate layer 3106. FIG. 31 also illustrates the bonding site 3108 after bonding. FIG. 31 also illustrates microchannel 3110, which was a 75-μm-wide polycarbonate microchannel. FIG. 32 also shows two polycarbonate layers 3203, 3204, and a polydimethylsiloxane (PDMS) layer 3206 as it deflects into microchannel 3208. With appropriate welding time and pressure the energy directors produce strong bonds. The PDMS compresses to create a conformal seal against the polycarbonate top.

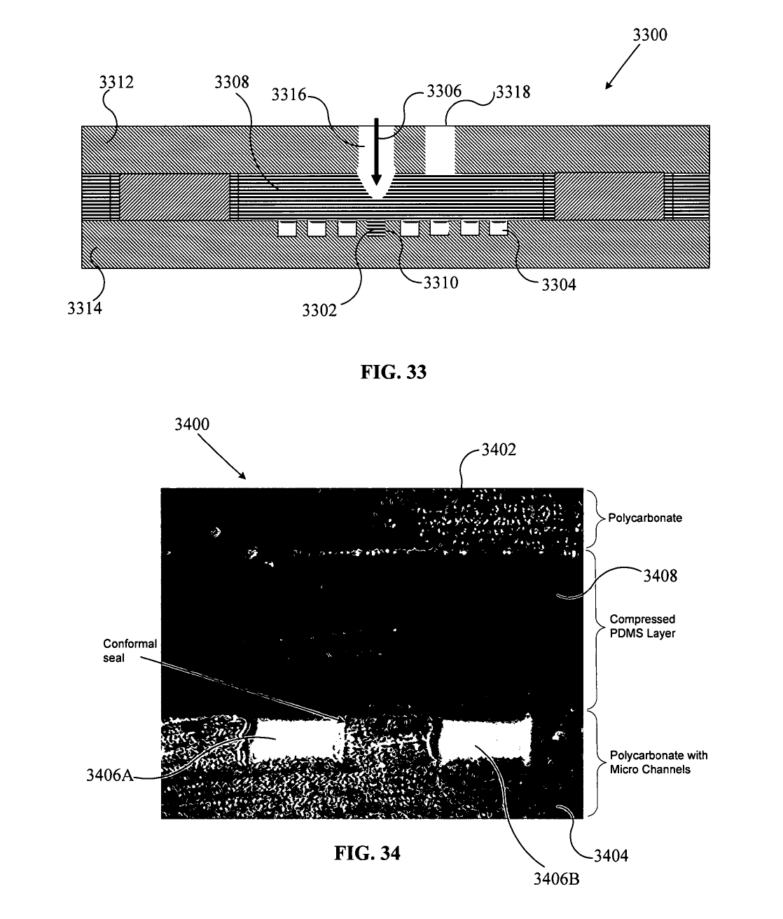

FIG. 33 illustrates a valve system 3300 comprising actuated valve 3302 and unactuated valves 3304. An actuation force, illustrated by arrow 3306, such as might be induced either pneumatically or hydraulically, compresses elastomeric layer 3308, which then deflects into microchannel 3310. The high modulus of layers 3312, 3314 constrain the elastomer of layer 3308 and allow for high actuation forces with minimal bulk material. This leads to less dead space above and below the valves 3302, 3304. In the case of pneumatic actuation the air channels can be closer together due to the higher rigidity of the walls. Compression of the elastomeric layer may be dependent on the welding pressure and time at a set energy level.

FIG. 34 is a photomicrograph illustrating a working embodiment of a microvalve system 3400 comprising a first polycarbonate layer 3402 and a second polycarbonate layer 3404 having plural microchannels 3406 produced therein. An elastomeric layer 3408, produced from PDMS, is provided that allows sealing of the microchannels 3406A, 3406B upon selective actuation. Microchannels 3406A, 3406B had a cross section of 100 μm wide×50 μm deep, which were sealed by a 270 μm thick PDMS layer compressed 43 μm by ultrasonic welding.

D. Microextractor

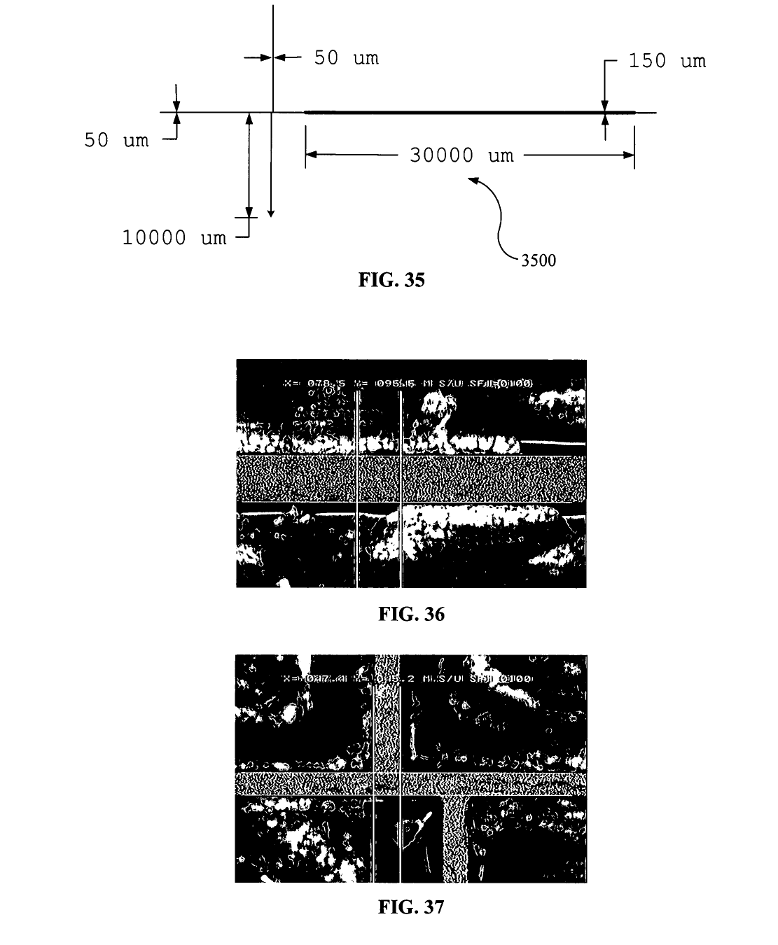

FIG. 35 illustrates one embodiment of a microextractor 3500. Microextractor 3500 provides three inputs and one output. Polycarbonate (PC) was chosen as the material for the initial embodiment since the material is readily available, easy to machine and compatible with solvents necessary for microreaction. Several microextractors have been produced in polycarbonate by various methods. One approach to microchannel fabrication has been simply planing the microchannel with a single point tool. This has worked well for a single microchannel but is difficult for producing the cruciform design in FIG. 35.

An alternative approach has been to produce the cruciform design via polymer micro hot embossing. The embossing process uses a vacuum hot press to pattern micro-features in a 750 μm thick polycarbonate film. Raised macro features and indented micro features have been formed side-by-side with fidelity of +/−3 μm. Ni-electroformed tools have been developed on stainless steel substrates to be used as embossing tools. The capability to fabricate 3.8 cm×6 cm electroformed embossing tools with feature sizes down to 50 um wide has been developed. The raised micro features are produced by electrodeposition of Ni onto rigid stainless steel substrates that are patterned photolithographically. The process is capable of thickness uniformity of +/−9% of the average feature height across the full tool substrate.

To avoid pitting during the electroforming process, the stainless steel substrate was constantly stroked with large air bubbles created by a plastic tube bubbler in the electroforming solution. The beaker which contains electroforming solution was placed in an ultrasonicator. Every 9 minutes, it was subjected to ultrasonic vibration for 1 minute until the electroforming process was done.

The height of the electroformed structures was ˜50 μm. This is very close to the height of the SU8 mold so that V-shaped irregularities that occurred were minimized to only 1˜2 μm. Such irregularities were further reduced to <1 μm after ashing the SU8 mold.

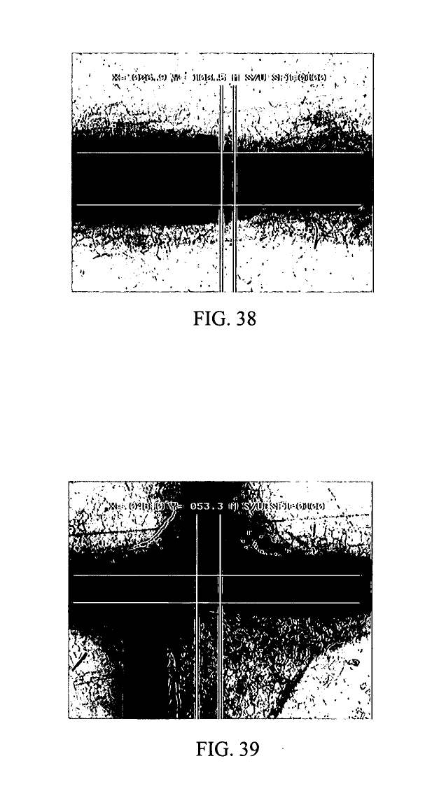

All in all, the electroforming technique was adjusted to yield optimized structures (see FIG. 36). The structure was then embossed onto polycarbonate substrate and mirror image of the structure was created (see FIGS. 38 and 39). This can be used as a building block for making other micro mixing and extracting devices.

E. Separators

During any synthesis that includes iterative steps, or different, plural steps, reactants, reagents and products likely will have to be separated from one another in order to provide an effective synthesis device. A number of different separation techniques have been developed for use in an integrated system. For example, methods have been developed for using dendrimers as templates for porous monolithic sorbents. Fused silica capillaries have been used as molds for the monoliths. A second approach comprises casting monoliths in situ in microfluidic channels on chips. A third approach involves using electrodes positioned on either side of a microchannel that are used for di-electrophoretic separations.

1. Sorbent-Based Chromatography or Solid Phase Extraction Unit

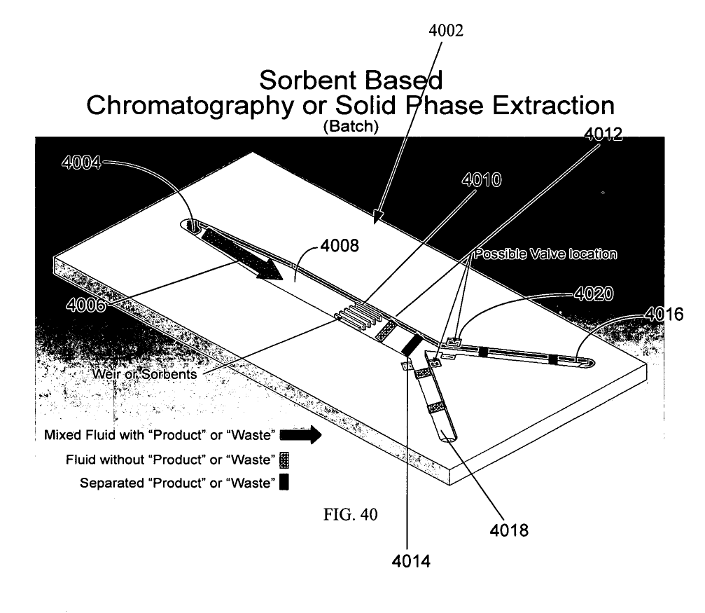

FIG. 40 illustrates one embodiment of a sorbent-based chromatography or solid phase extraction unit 4002. Extraction unit 4002 includes a first fluid port 4004 for receiving a mixed fluid stream 4006, such as a stream comprising product and waste materials. Fluid stream 4006 flows through microchannel 4008. Extraction unit 4002 includes a weir and/or at least one sorbent material, and potentially plural weirs and/or sorbent materials, 4010. Mixed product stream 4006 is then effectively separated into a first separated stream 4012 and at least a second separated stream 4014. These two separated streams 4012 and 4014 then can flow intermittently through microchannel 4008. Alternatively, the illustrated embodiment of extraction unit 4002 includes Y microchannels 4016 and 4018. These two microchannels 4016 and 4018 can be used to separate the two flowing separated fluid stream 4012 and 4014. Separation into Y microchannels 4016 and 4018 can be facilitated by locating valves prior to the Y microchannels, such as at or about position 4020.

2. Capillary Chromatography

Capillary electrochromatography (CEC) is a rapidly growing area in analytical separations. Monolithic columns are perhaps the most attractive alternative to conventional packed columns for liquid chromatography (LC) and CEC. An in situ polymerization process can be performed directly within the confines of a mold, typically a segment of capillary tubing or a channel on a microchip. Both silica and organic-based monolithic columns are known. This procedure provides a sorbent for which frit formation and irreproducible packing are no longer issues.

The porosity of the polymeric stationary phase in monolithic columns is usually dictated by the nature and amount of the porogenic solvent employed. Aside from affecting porosity, adjustments of the amount and nature of the porogenic solvent(s), alter other properties such as the surface area, nature and swelling properties of the resulting monoliths.

Recently, Chirica & Remcho (Chirica, G. S., Remcho, V. T. J. Chromatogr. A 2001, 924, 223-232, incorporated herein by reference) described a new synthetic method for preparing monoliths with porosity dictated by the size of spherical silica particle templates. In addition to tailoring the pore size, this method offers the ability to influence the surface characteristics of the finished polymer by employing silica beads with specific surface chemistry.

New monolithic stationary phases that afford control over porosity and, to a certain degree, over the surface chemistry of the sorbent have been considered. The novelty of this approach lies in the use of dendrimers for generating uniform pore structures. PAMAM dendrimers, unlike classical polymers, have a high degree of molecular uniformity, narrow molecular weight distribution, specific size and shape characteristics, and a highly-functionalized terminal surface.

3. Fused-Silica Tubing Capillaries

a. Chemicals and Materials

Butyl methacrylate (BMA), ethylene dimethacrylate (EDMA), 2-acrylamido-2-methyl-propansulfonic acid (AMPS), 2,2′-azobisisobutyronitrile (AIBN), Starburst (PAMAM) dendrimer (generation 4.5; 10% solution in methanol), and [(methacryloxy)-propyl] trimethoxysilane were purchased from Aldrich (Milwaukee, Wis., USA) and used as received. The solvents employed in the CE and CEC runs were HPLC grade and were purchased from Fisher Scientific (Pittsburgh, Pa., USA). Fused silica tubing of 100 μm I.D.×375 μm O.D. was purchased from Polymicro Technologies (Phoenix, Ariz., USA).

b. Production of Lysozyme Digest

Chicken egg lysozyme (Aldrich) was dissolved in 20 mM ammonium bicarbonate (pH 7.8) and digested using modified trypsin (Aldrich) (0.5 μg/mL) for approximately 72 hours at 37° C.

c. Instrumentation

Electrochromatographic experiments were carried out using an Agilent/HP^{3D}CE (Waldbronn, Germany) instrument, modified such that pressure of up to 12 bar can be applied on the inlet and/or outlet vials. Data acquisition and processing were performed with the Agilent ChemStation software. Samples were injected electrokinetically (5 kV for 3 sec). Pressure injection (50 mbar for 3 sec) was also used occasionally. The cassette temperature was set at 22° C.

Capillary columns during monolith preparation were examined with a simple Stereomaster optical microscope (Fisher Scientific, Houston, Tex., USA) with 40× magnification. The column morphology was studied using an AmRay (Bedford, Mass., USA) scanning electron microscope (SEM) operated at 10 kV.

d. Column Preparation

i. Pretreatment of the Capillary

For columns in which the monolith was anchored to the fused-silica capillary wall, functionalization of the walls was required. The fused-silica tubing was derivatized with [(methacryloxy)-propyl] trimethoxysilane, using a method developed by Hjertèn (Hjertèn, S. J. Chromatogr. 1985, 347, 191-195, incorporated herein by reference). Briefly, the capillary was flushed with a solution of sodium hydroxide (1 M) followed by water for at least 30 minutes each. The capillary was filled with a 4:1 (monomer/solvent; v/v) solution of [(methacryloxy)-propyl] trimethoxysilane and 6 mM acetic acid. The solution was kept in the capillary for at least 1 hour. The capillary was flushed with water for several minutes and finally emptied and dried with a flow of nitrogen.

ii. Monolithic Column Preparation

AIBN (1 wt % with respect to the monomers) was dissolved in a monomer mixture consisting of 40% EDMA, 59.7% BMA and 0.3% AMPS. The solvent, methanol, was slowly admixed to the monomers in a 2:3 (v/v) ratio. Aliquots of 1 mL of this mixture were added to several vials containing specific amounts of Starburst (PAMAM) dendrimer. The dendrimer, commercially available as a 10% solution in methanol, was used after the removal of methanol by vacuum distillation. After addition of the monomer solution, the homogeneous mixtures were purged with nitrogen for 10 minutes. The capillary was filled with the polymerization mixture using a 100 μL syringe. Both ends of the capillary were sealed with rubber septa, and the column was submerged in a 60° C. bath for 20 hours. Using a syringe pump, the resulting monolith was washed with the mobile phase to flush out the residual reagents, dendrimers and methanol. With appropriate rinsing solutions, dendrimer templates can be recovered and reused.

Using a small piece of PTFE tubing the monolithic column was joined to a fused-silica open tube onto which a detection window was burned.

In addition, selected polymers were prepared in “bulk” quantities. These polymers were ground and then washed with the mobile phase to remove the dendrimers and any residual reagents. After drying, the porosity of the polymers was determined by mercury intrusion porosimetry.

iii. Physical Characterization of the Monoliths

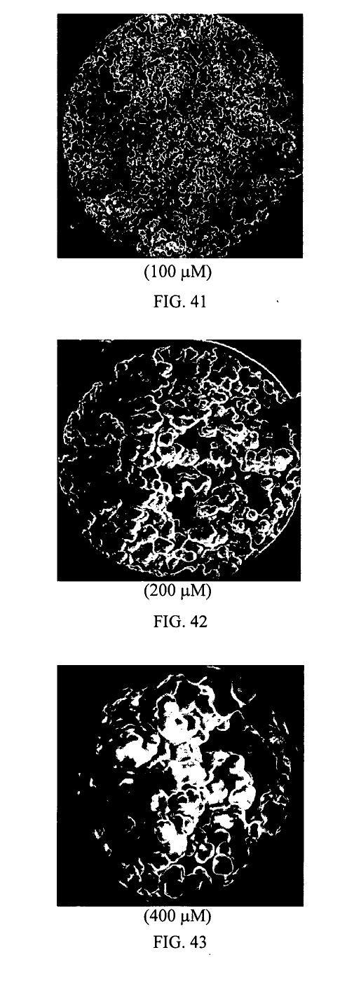

The monoliths that were not anchored to the capillary walls were extruded from the capillary and used for morphologic characterization of the monoliths. The polymer was sputter-coated with gold and examined with a scanning electron microscope. The SEM images presented in FIGS. 41-43 demonstrated that this procedure renders a highly permeable monolith with porosity dictated by the dendrimer concentration.

The structures of the various monolithic columns differ significantly, and depend on the dendrimer concentration in the polymerization mixtures. At very high concentrations of dendrimer (such as 400 μM or greater), the microglobules become larger and the globule stacking and the channel distribution become less uniform. This is likely the causative factor behind the decrease in column efficiency and resolution achieved at the highest dendrimer concentrations studied.

The polymeric monoliths preferably are highly permeable for their application as sorbents in extractions and chromatographic separations. Different column porosities were obtained by varying the amount of the dendrimer template.

Porosity data and the pore size distribution profiles of the dried monoliths were obtained by mercury intrusion porosimetry. These analyses were performed by Micromeritics Instrument Corporation (Norcross, Ga.) using a Micromeritics AutoPore mercury porosimeter.

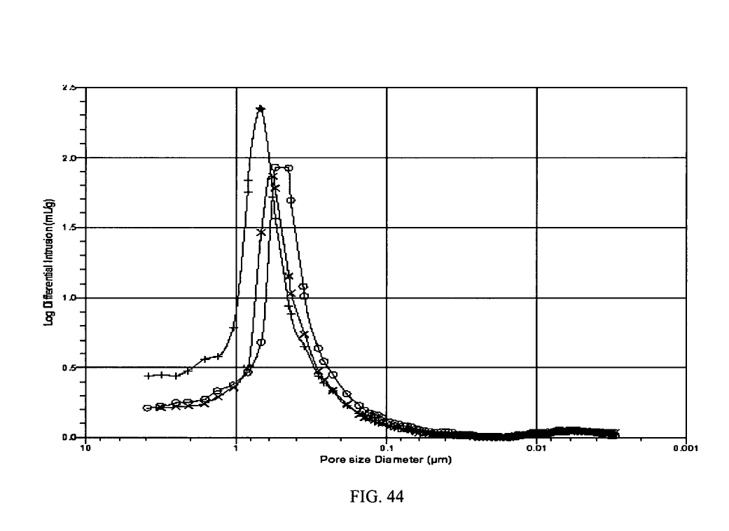

FIG. 44 shows the differential pore size distribution profiles for several porous polymers prepared using different dendrimer template concentrations. There is a noticeable difference between pore size distribution profiles for these columns. For instance, the mode pore diameter (the pore diameter at the maximum of the distribution curve) increases from 600 nm for column 1 (produced in the absence of dendrimers) to 700 nm for column 2 (50 μM dendrimers) and reaches 800 nm for column 3 (100 μM dendrimers). Based on these results, the average pore size of the monoliths can be adjusted as desired by selecting the dendrimer template concentration.

e. Chromatographic Characterization of the Monolithic Columns

The peak achievable efficiency of monolithic columns was examined in the CEC mode by measuring the peak width at half height for toluene in order to investigate the effect of dendrimer concentration on chromatographic performance.

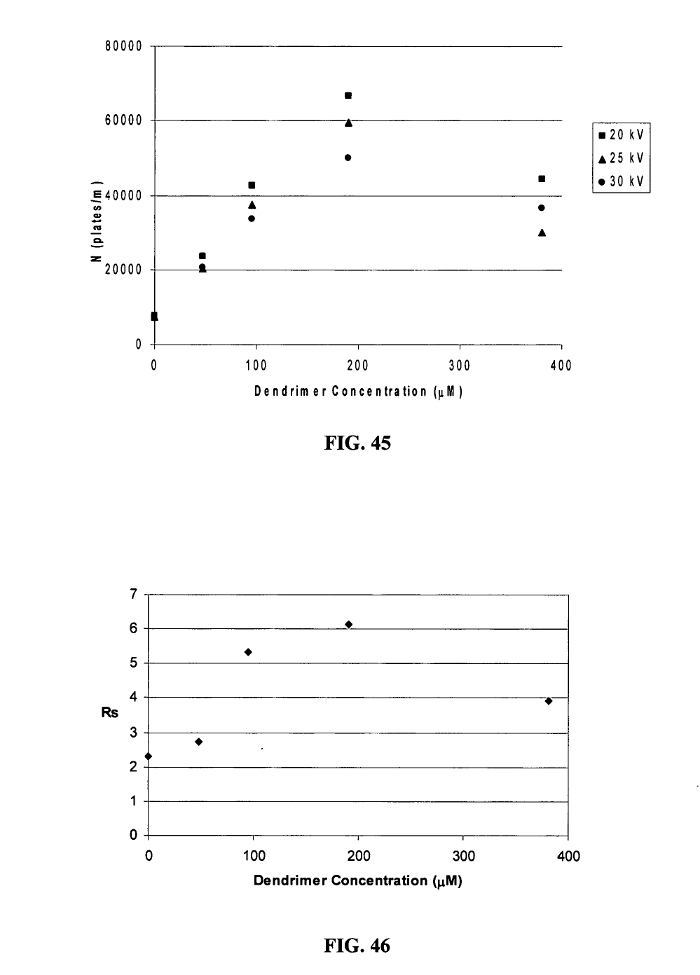

FIG. 45 shows a plot of efficiency as a function of dendrimer template concentration. As dendrimer concentration increases from 0 to 400 μM, the column efficiency increases from about 8,000 to 60,000 plates/m, reaching a maximum at 200 μM dendrimer concentration. As observed in the SEM data, at 400 μM dendrimer concentration, the pores become larger and less uniform, which is likely the cause of the decrease in column efficiency. The large size of the pores results in a smaller surface area and a larger total eluent volume; consequently the analyte is less retained and experiences greater diffusional relaxation, hence the lower efficiency.

Another parameter used to evaluate the chromatographic performance of these monolithic columns was resolution in the separation of acetone and toluene. As shown in FIG. 46 and as anticipated, the dendrimer concentration had a similar effect on chromatographic resolution as on efficiency. The resolution increases with the dendrimer concentration and reaches a maximum at 200 μM dendrimer concentration. Again, a decrease in column performance was observed at 400 μM dendrimer concentration.

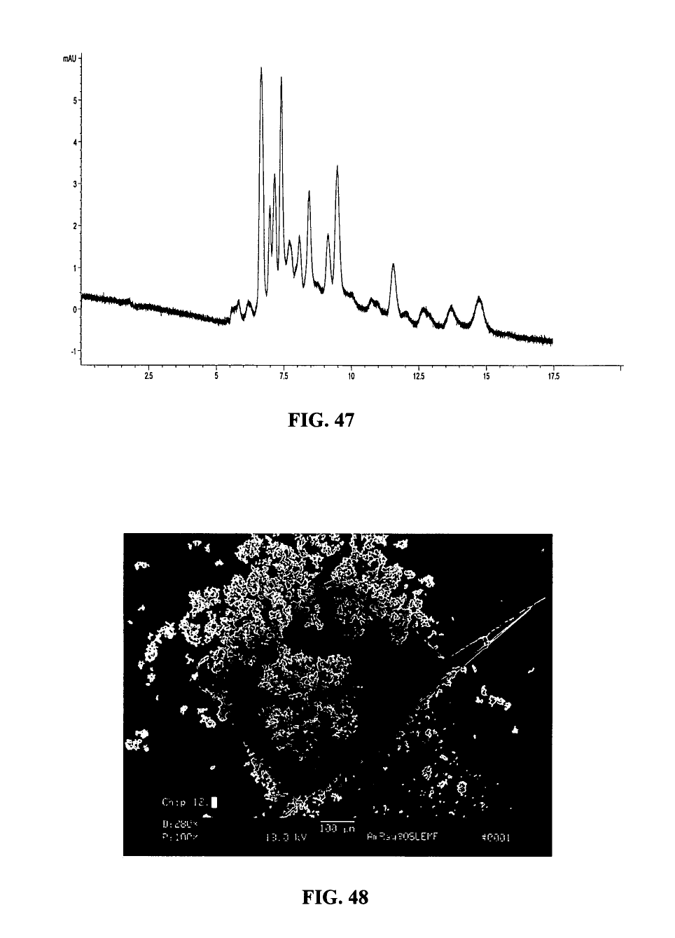

The performance of monolithic columns has mostly been evaluated using small, neutral organic molecules, which are typically separated under conditions of reverse-phase chromatography. To extend the range of monolithic column applications, the separation of lysozyme tryptic digest fragments was attempted on a column prepared using 50 μM dendrimer template. The chromatogram shown in FIG. 47, obtained using 40/60 ACN/40 mM phosphate buffer (pH=2) at an applied voltage of 10 kV, demonstrates the ability of these columns to separate complex mixtures.

f. Dendrimer-Templated Sorbents in Microfluidic Devices

Dendrimer-templated monoliths as described above are being immobilized in polycarbonate (PC) microfluidic chips. The chips are prepared by vacuum hot embossing a PC blank on a nickel-electroformed master, then sputtering the PC chip with silica in a commercial sputtering apparatus. The thin film of silica on the PC surface is then functionalized with an anchoring group and the dendrimer-templated monolith is cast as described above for fused silica capillaries.

Thus, a new class of porous polymer monoliths has been developed for use in a new format for capillary electrochromatography. The porosity of monoliths has been varied by adjusting the amount and nature of the porogenic solvent or by incomplete polymerization, although a macromolecular template PAMAM dendrimer is now an alternative method for pore generation. Structural attributes of this template allowed for production of continuous polymeric rods exhibiting uniform porosity.

As indicated above, organic-based columns also have been prepared. For purpose of rapid prototyping, a straight channel master was manufactured from a Teflon sheet using a CNC milling machine. Sylgard 184 PDMS prepolymer was mixed thoroughly in a 10:1 mass ratio of silicone elastomer to curing agent, degassed and poured onto the master. Chips were cured at 65° C. for 24 hours. After curing, the PDMS replica was peeled from the mold and holes were punched into the polymer to create access ports. Flat PDMS substrates were obtained by casting prepolymer mixture against a clean glass plate and curing. The two pieces of PDMS were placed in an oxygen plasma and oxidized for 1 minute. When joined together, the oxidized parts sealed irreversibly. Immediately after plasma oxidation and sealing of the upper and lower portions of the chip, the microchannel produced between the two layers was derivatized with [(methacryloxy)-propyl]trimethoxysilane. Following derivitization of the channel surfaces, monolithic porous polymers were prepared in situ on the microchip by photoinitiated polymerization.

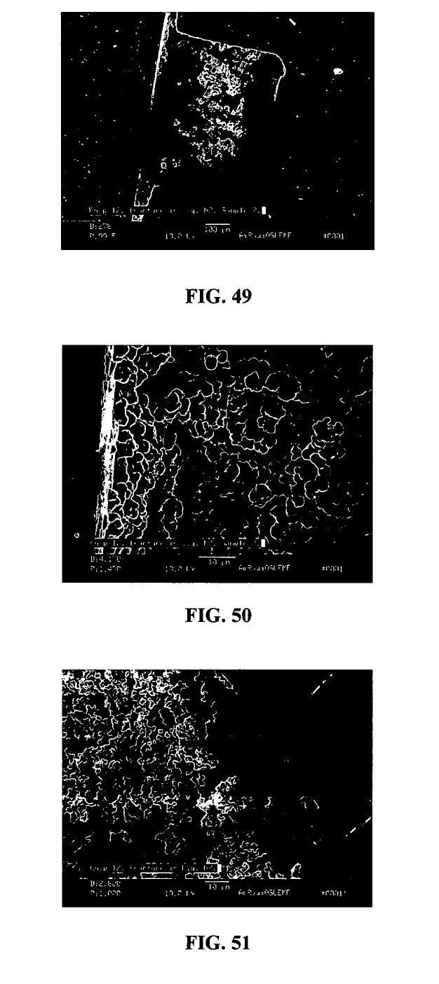

The elasticity of the PDMS made it difficult to obtain a clean cut of the microchip and damaged the monolith. FIGS. 48 and 49 present the channel cross sections of a chip fractured at room temperature and after being frozen in liquid nitrogen, respectively. The monolith near the channel wall is adhered well to the wall surface. Aside from this, the SEM images presented in FIGS. 50 and 51 indicate a uniform structure of the monolith close to the walls, similar to that in the bulk material.

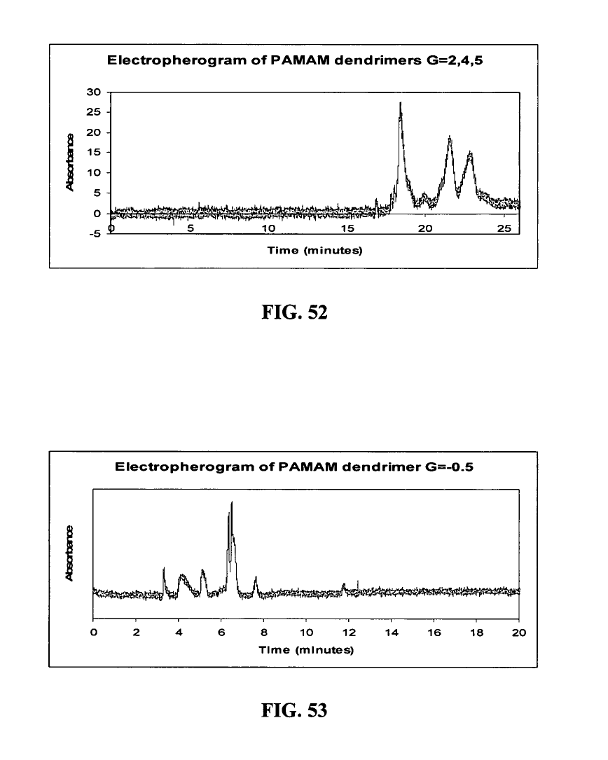

Capillary zone electrophoresis, CZE, utilizes the open, non-derivatized channels, such as those with an SiO₂ channel. The separation results typically obtained for dendrimer separations using CZE are shown in FIG. 52. A capillary electrochromatography, CEC, method (typical results shown in FIG. 53) are obtained using a porous monolithic polymer anchored to the SiO₂ walls and filling the channel. The CZE method are amenable to operating a microchemical nanofactory in a continuous mode, whereas the CEC method is likely best used in a pulsed or quasi-continuous mode.

With a CEC method the eluted dendrimer and precursors will be detected in-channel following separation using indirect laser-induced fluorescence. This method relies on an eluent solution matrix containing a uniform concentration of a background fluorophore. The eluate, the dendrimer or precursors, will cause a decrease in concentration of this background fluorophore resulting in a decrease of detected signal. This decrease in signal can provide quantitative analysis of concentration of the eluate or simply serve as a photogate for valve timing. With a CZE method the same detection method can be used.

4. Particle Separation

a. Field-Flow Fractionation

It may prove to be difficult to separate nanoparticles due to aggregations effects, low DEP susceptibility, etc. In this event, Field-Flow Fractionation (FFF) may prove a valuable option. FFF is a separation technique applicable to macromolecules, colloidal materials, and particles up to tens of microns in diameter. It is a zonal elution technique in which separation takes place in a thin, open channel across which a field is applied. Like DEP, it is a single-phase technique that is conducted in batch-mode but which has the potential for continuous-mode operation (called SPLITT fractionation). Retention in the channel occurs when sample materials interact with the field and are driven into slower streamlines close to a bounding wall. In the “normal” mode of elution, relevant for macromolecules and colloids, a steady state, transverse concentration distribution results from transport toward the wall by field interaction, and back-diffusion from the region of higher concentration. The thickness of this steady state distribution within the fluid velocity profile determines the elution velocity of the zone. In the “steric” mode of elution, the size of the particles principally determines their elution rate. The larger particles protrude further from the wall into the faster flowing streamlines and elute before smaller particles.

Hydrodynamic lift forces influence retention in the steric mode. In the life sciences, these result in what is known as the “tubular pinch effect” shown by the tendency of blood cells to be driven away from vessel walls. These lift forces can be exploited to separate particles having the same size but differing in the strength of their interaction with a field—an alternative to a DEP method. Those that interact more strongly are driven closer to the bounding wall, and elute more slowly.

b. Asymmetric Flow Field-Flow Fractionation.

Asymmetric Flow Field-Flow Fractionation (AFl-FFF) is an FFF variant by which it is possible to separate polymers and particles ranging from about 1 nm to a few micrometers. Compared to other FFF methods, AFl-FFF is more universal and efficient with a broader application range.

Separation in conventional FFF occurs in a thin rectangular flow channel, which is comparable to the separation column used in chromatography. In general this channel is 30 cm long, 4 cm wide and 250 μm tall. The channel flow, of an aqueous or organic solvent, carries the sample through the channel. Because of the low channel height this flow is laminar.

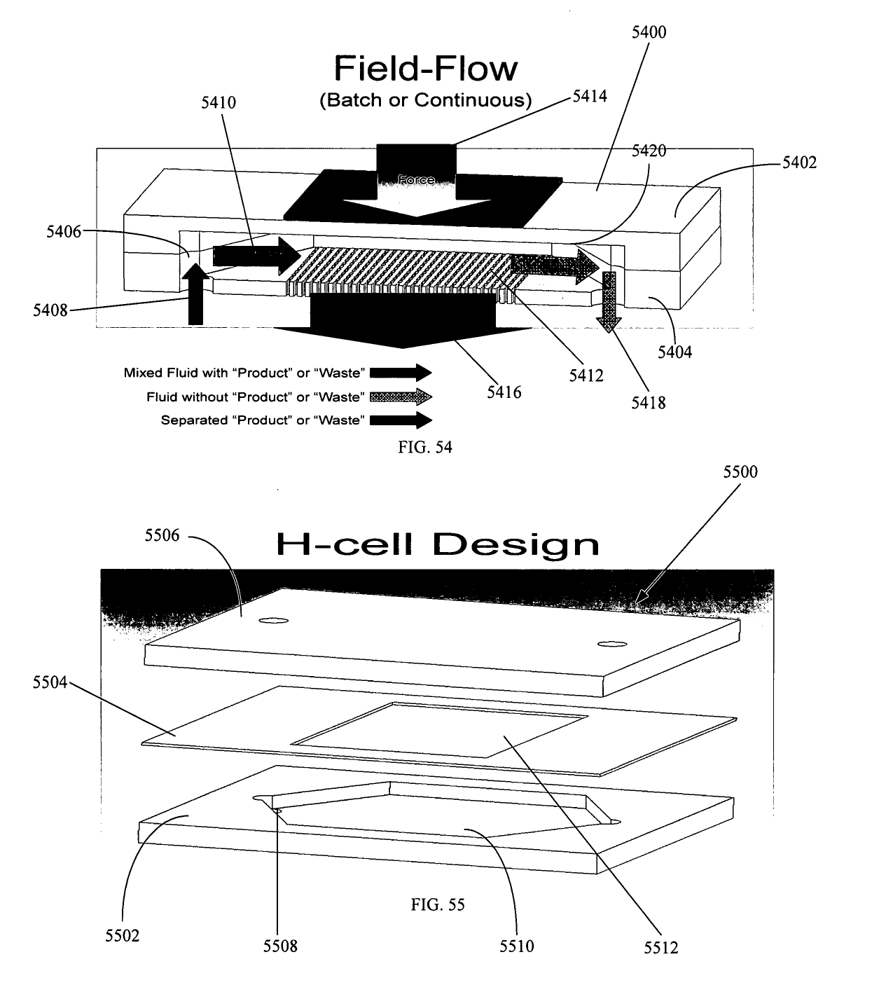

FIG. 54 illustrates one embodiment of an AFl-FFF separator 5400. Separator 5400 includes a first lamina 5402 and a second lamina 5404. A fluid aperture 5406 is formed in lamina 5404 to receive a fluid flow 5408. Fluid 5408 may be a mixture of materials, such as product and “waste” materials, such as side reaction products or unused reactants. Fluid 5408 flows into microchannel 5410 and over a region 5412 in which the lower (accumulation) wall is porous. Porous region 5412 may be a frit topped by a porous membrane. This region also may taper towards a fluid outlet. Perpendicular to this fluid flow a second force, indicated as 5414, is generated. In AFl-FFF a “cross flow” is used for generating the second force field, thus the technique is capable of separating particles based on differences in their size (which dictates their susceptibility to the cross-flow). An increasing downforce 5414 is thus induced and particles accumulate more rapidly as they migrate down the channel. Differently sized particles with varying diffusion coefficients are separated within the velocity gradient inside the channel. Particles or polymers are forced in the direction of the lower membrane by the cross flow. The cross flow leaves the channel through this membrane, whereas particles and polymers are retained above the membrane. Smaller particles will diffuse back into the channel further than larger particles because of their larger diffusion coefficients. As a result, smaller particles are located in the area of faster flow streamlines and in this way are eluted from the channel ahead of larger particles. With reference to FIG. 54, a first separated stream 5416 is formed, as is a second separated stream 5418, resulting in mixture separation or purification of the mixed fluid stream 5408. By virtue of having only one porous wall, AFl-FFF devices are easier to fabricate than conventional Fl-FFF devices.

For the AFl-FFF devices, the upper channel wall 5420 is impermeable and will be made of polycarbonate. Cross-flow may be generated by dividing laminar inlet flow into two components: one being the laminar bulk flow and the other being the cross-flow component that exits the channel at the depletion wall. Region 5412 may be made by perforating the floor of the trapezoidal polycarbonate lower layer using a laser via drill currently available at the ONAMI fabrication facility. This asymmetric cross flow design offers the advantage of greatly simplified, and the added advantage of increased fluidic simplicity: only one inflow pump is needed, and this can be the fluid delivery pump that currently drives the microreactor further upstream. Additional valves, a precise flow-measuring control unit and electronics for the automated operation of the system (not illustrated) can be used as well.

5. H-Cell Separators

FIG. 55 illustrates one embodiment of an H-cell 5500. The illustrated H-cell 5500 includes a bottom lamina 5502, a middle lamina 5504 and a top lamina 5506. Bottom lamina 5502 defines fluid flow aperture(s), such as aperture 5508. Bottom lamina 5502 also defines a fluid receiving region 5510. Middle lamina 5504 also defines a flow aperture, such as aperture 5512.

6. Evaporative H-Cell Separators

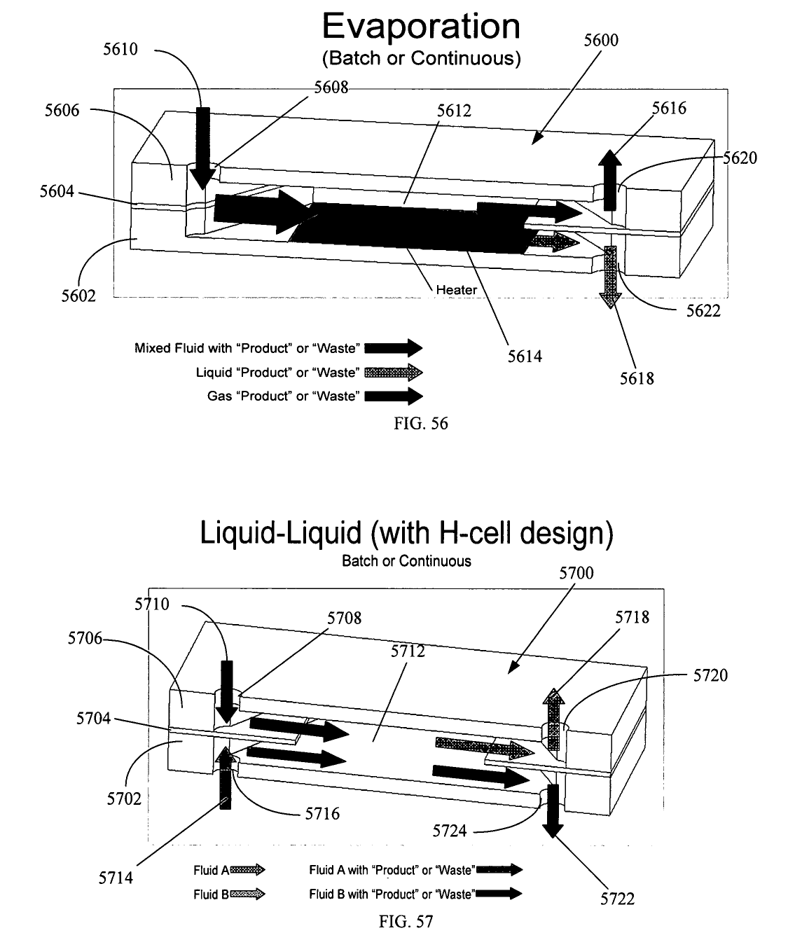

FIG. 56 illustrates one embodiment of an H-cell evaporative separator 5600. The illustrated H-cell 5600 includes a bottom lamina 5602, a middle lamina 5604 and a top lamina 5606. Top lamina 5606 defines fluid flow aperture(s), such as aperture 5608 for receiving a mixed fluid flow 5610. Mixed fluid flow 5610 flows into and through microchannel 5612, which includes a heater 5614. Mixed fluid flow 5610 includes components having sufficiently different boiling points such that a first component evaporates to produce a gas flow 5616, leaving a less volatile liquid phase component 5618. Gas flow 5616 can be removed through gas flow aperture 5620. Any remaining liquid phase component 5618 likewise can be removed from separator 5600 through liquid flow aperture 5622.

7. Liquid-Liquid H-Cell Separators

FIG. 57 illustrates one embodiment of an H-cell separator 5700. The illustrated H-cell 5700 includes a bottom lamina 5702, a middle lamina 5704 and a top lamina 5706. Top lamina 5706 defines fluid flow aperture(s), such as aperture 5708 for receiving a mixed fluid flow 5710. Mixed fluid flow 5710 flows into and through microchannel 5712. A second fluid stream 5714 also flows into microchannel 5712 through fluid inlet aperture 5716. The two fluid flows 5710 and 5714 then are flowing through microchannel 5712 sufficiently adjacent each other to provide diffusional flow between the two fluid streams. This produces a first separated fluid stream 5718, which flows out of the separation device 5700 through fluid exit port 5720. A second separated fluid stream 5722 also is produced, which flows out of the separation device 5700 through fluid exit port 5724.

8. Counter Current Liquid-Liquid H-Cell Separators

FIG. 58 illustrates one embodiment of an H-cell separator 5800. The illustrated H-cell 5800 is useful for counter current flow, whereas the embodiment of FIG. 57 contemplates fluid flow in the same direction. Separation unit 5800 includes a bottom lamina 5802, a middle lamina 5804 and a top lamina 5806. Top lamina 5806 defines fluid flow aperture(s), such as aperture 5808 for receiving a mixed fluid flow 5810. Mixed fluid flow 5810 flows into and through microchannel 5812. A second fluid stream 5814 also flows into microchannel 5812 through fluid inlet aperture 5816. Miced fluid flow 5810 flows in a counter current direction to fluid stream 5814. The two fluid flows 5810 and 5814 then are flowing through microchannel 5812 sufficiently adjacent each other to provide diffusional flow between the two fluid streams. This produces a first separated fluid stream 5818, which flows out of the separation device 5800 through fluid exit port 5820. A second separated fluid stream 5822 also is produced, which flows out of the separation device 5800 through fluid exit port 5824.

9. Fluid-Fluid Y Separators

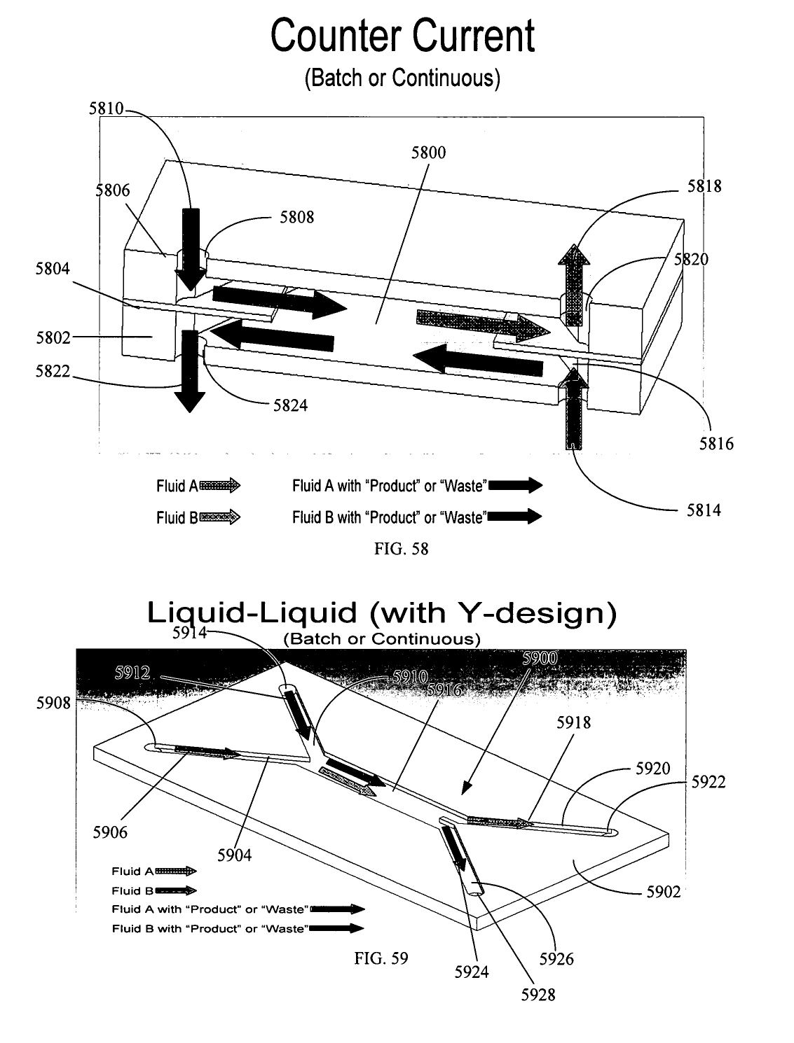

FIG. 59 illustrates one embodiment of a fluid-fluid, typically a liquid-liquid, Y separator 5900. The illustrated embodiment of the separator has a single lamina 5902. A person or ordinary skill in the art will appreciate that the Y separator may include plural laminae. Y separator 5900 includes a first microchannel 5904 for receiving a first fluid flow 5906 through fluid flow aperture 5908. Y separator 5900 includes a second microchannel 5910 for receiving a second fluid flow 5912 through fluid flow aperture 5914. First fluid stream 5906 and second fluid stream 5912 then flow into microchannel 5916. The two fluid flows 5906 and 5912 then are flowing through microchannel 5916 sufficiently adjacent each other to provide diffusional flow between the two fluid streams. This produces a first separated fluid stream 5918, which flows into Y microchannel 5920 and out of the separation device 5900 through fluid exit port 5922. A second separated fluid stream 5924 also is produced, which flows into Y microchannel 5926 and out of the separation device 5900 through fluid exit port 5928.

10. Precipitation ¦Separators¦_{[bkp2]}

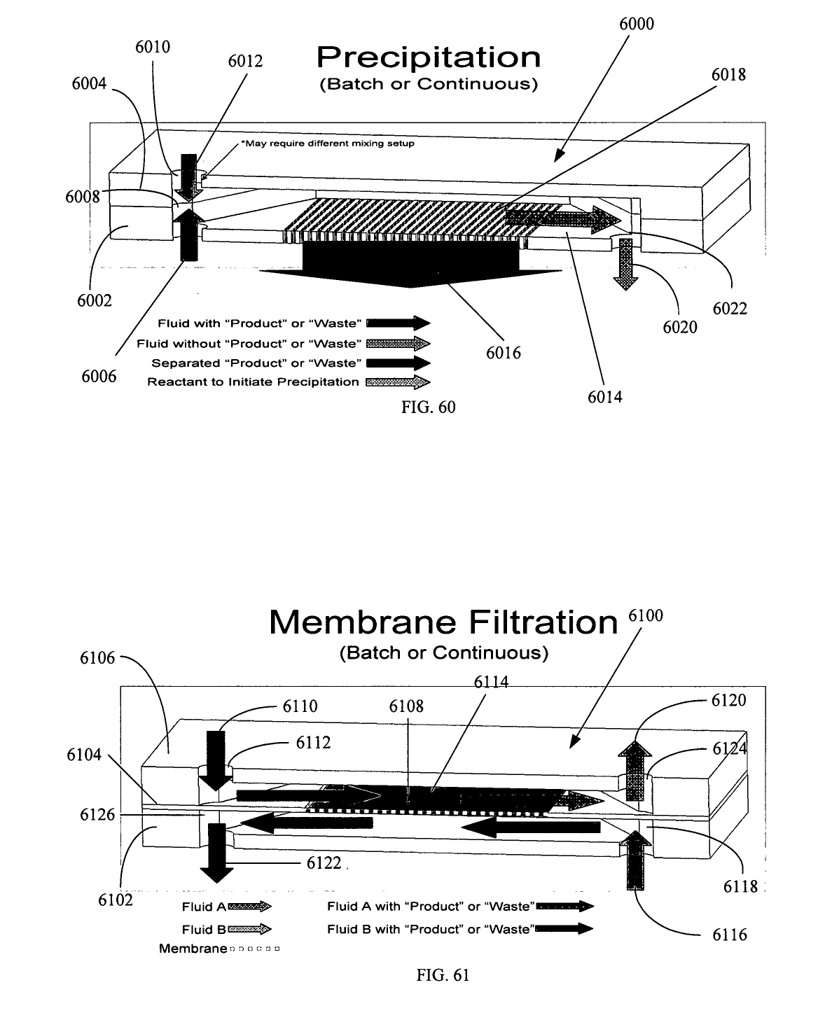

FIG. 60 illustrates one embodiment of a precipitation separator 6000. Separator 6000 has a first lamina 6002 and a second lamina 6004. A first mixed fluid stream 6006 is introduced to separator 6000 via fluid inlet aperture 6008. If necessary, a second material stream 6010 can be introduced to separator 6000 via inlet aperture 6012. Fluid stream 6006 and second material stream 6010 flow in and through microchannel 6016. The residence time in the microchannel is sufficient to provide for precipitation of some moiety in the streams 6006 and 6010. Upon precipitation, the precipitate 6016 flows out of the separation 6000 through porous portion 6018. A separated fluid stream 6020, devoid or at least substantially devoid, of the precipitate 6016 then also exits separator 6000 through outlet aperture 6022.

11. Membrane Separators

FIG. 61 illustrates one embodiment of a membrane separation unit 6100. Separator 6100 has a first lamina 6102, a second lamina 6104 and a third lamina 6106. Separator 6100 also includes a membrane 6108. Membrane 6108 may have variable properties and chemical composition, and is selected by determining the materials that need to be removed from a mixed stream. A person of ordinary skill in the art will understand how to select suitable purification membranes based on the separation required for a particular system. A first fluid stream 6110 enters separator 6100 via fluid inlet aperture 6112 and flows into and through microchannel 6114. A second fluid stream 6116 enters separator 6100 through fluid inlet aperture 6118, and the second stream 6116 thus enters microchannel 6114. Both fluid stream 6110 and 6116 flow through the microchannel 6114 in a manner effective to contact membrane 6108. Membrane 6108 might be, for example, a semiporous membrane that allows certain materials to pass while excluding others. Thus, such materials are removed from a first stream, such as fluid stream 6110, pass through membrane 6108 and enter second stream 6116. Thus, two new fluid streams are produced, 6120 and 6122. Each of these separated streams then exit separator 6100 through fluid exit ports 6124, 6126, respectively. The illustrated embodiment of separator 6100 is a counter current flow separator, but this may not be necessary, and hence a person of ordinary skill in the art will understand that co-current flow membrane separation embodiments also are within the scope of the present invention.

12. Size Exclusion Chromatograph Separators

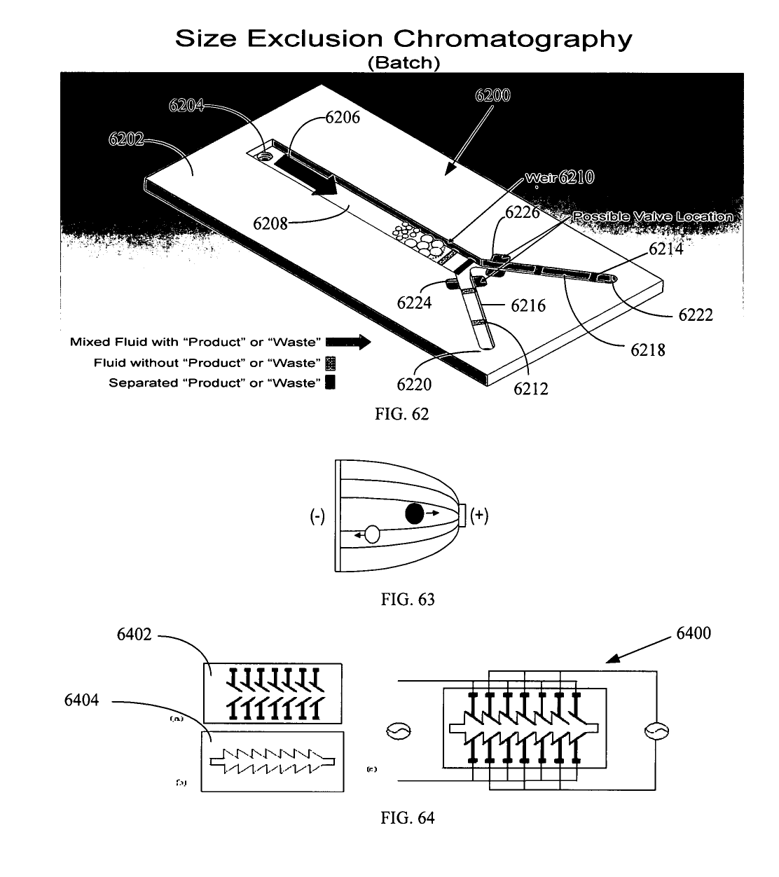

FIG. 62 illustrates one embodiment of a membrane separation unit 6200. Separator 6200 is illustrated as having only a single lamina 6202, although the illustrated structure, and similar structures useful for performing size exclusion chromatography, also can include plural lamina. Separator 6200 includes a fluid inlet aperture 6202. A mixed fluid stream 6204 flows into separator 6200 through inlet aperture 6202, and enters and flows through microchannel 6208. A size exclusion weir 6210 is positioned in microchannel 6208. Fluid flow stream 6204 encounters size exclusion weir 6210, and material separation then occurs based on material size to form size separated streams 6212 and 6214 that flow through Y channels 6216, 6218, respectively, and exit separator 6200 via flow exit apertures 6220, 6222, respectively. Separation of the size separated streams 6212 and 6214 can be facilitated by using valves 6224 and 6226.

13. Dielectrophoretic Particle Separation

FIG. 63 illustrates one method for separating particles based on positive and negative dielectrophoresis. FIG. 64 illustrates one embodiment of a working device 6400 that used DEP for particle separation. Device 6400 includes an upper electrode array defining lamina 6402, and a second lamina 6404 that defines a ratchet-shaped microchannel 6406. Particles flowing into device 64 are influenced by either a positive or negative DEP, and are separated as such particles progress through the microchannel 6406.

a. Dielectrophoresis

Dielectrophoresis (DEP) is a separation method in which particles are segregated according to their susceptibility to a non-uniform electric field. A non-uniform electric field is generated by applying voltage across electrodes of appropriate geometry or by placement of insulating posts between a pair of electrodes. In both cases, the components are configured to spatially distort the electric field. Unlike electrophoresis, where only dc voltage is used, either dc voltage or an ac waveform can be used in DEP to discriminate between different particles in a sample. By varying the frequency of the applied voltage, it is possible to induce a dipole moment in a particle and thereby cause the particle to experience a positive or negative dielectrophoretic moment and cause the particle to move into a region of high potential or low potential, respectively. DEP is a batch-mode, single-phase technique that has demonstrated capability for adaptation to continuous-mode application.

Initial devices used to produce non-uniform electric fields were constructed by placing a wire in the center of a glass tube in which another wire was wrapped along the inner wall of the glass tube³⁷. These devices required high potentials and were limited to analysis of particles 1 μm in diameter or larger due to Joule heating effects, which led to Brownian movement that countered the dielectrophoretic force. Benefits in decreasing the scale of dielectrophoretic devices, thereby increasing the dielectrophoretic force, have been discussed by Bahaj and Bailey, who derived the following scalar relation:

$${F}_{DEP} ∝ \frac{( {V}^{2} )}{( {L}_{e}^{3} )} \qquad ( 1 )$$

where F_{DEP} is the dielectric force, V is the applied voltage and Lₑ is the distance between electrodes. From Eq. (1), it can be seen that F_{DEP} is inversely proportional to the cube of the dimensions of the electrodes used, so by miniaturization of DEP devices the magnitude of the dielectrophoretic force exerted on a particle is increased. Another finding was that decreasing electrode size led to a reduction in Joule heating.

With the use of semiconductor manufacturing technologies as discussed herein (lithography, electron beam writing, laser ablation, nanoimprint lithography, vacuum hot embossing, etc.) a move towards device miniaturization is occurring. Benefits of device miniaturization include decreased consumption of reagent, reduced working time, and the possibility of integrating DEP systems into working production devices such as microreactors.

Several different modes of microchip-based DEP exist, including focusing/trapping-, isomotive-, and traveling wave-DEP. One disclosed embodiment concerns “conventional” DEP in the microchip format: focusing and trapping of particles in devices that utilize electrode arrays and arrays of insulating posts as the geometries. Here, separation of particles is made possible through polarization of a particle relative to its medium, followed by transport of the particle through the medium. This is an inherently rapid, single-phase process, and thus is quite suitable for our application to microreactor-based production of Au nanoparticles. DEP device 6400 is useful for separating nanoparticles and can be integrated with other unit operations, such as planar microreactor, to form a nanofactory.

Estimation and determination of particle mobility. The real component of the Clausius-Mossotti factor accounts for the polarization of a particle relative to its suspending medium, and it is this induced dipole that dictates the direction a polarized particle will move in a non-uniform field. Since the movement of a dielectric particle is mitigated by the complex permittivities of the particle and suspending medium, it is possible to discriminate between particles based on their polarizabilities.

Determination of the type of dielectrophoretic moment a particle will experience can be accomplished by calculating Re[K(ω)] using equation (2). Re[K(ω)] refers to the real component of the Clausius-Mossotti factor³⁷ which is found by taking the real component of:

$$K ( ω ) = \frac{( {ɛ}_{p}^{*} - {ɛ}_{m}^{*} )}{( {ɛ}_{p}^{*} + 2 {ɛ}_{m}^{*} )}$$

(2) where ∈ₚ* and ∈ₘ* are the complex permittivity of the particle and medium respectively, and ∈*=∈−jσ/ω where ∈ is the permittivity, j is √−1, σ is the conductivity, and ω is the angular frequency of the applied electric field.

The Clausius-Mossotti factor is frequency dependent, as it is determined from the frequency dependent complex permittivities of the particle and the medium^{37,40,41}. As such, by constructing a plot of the real component of the Clausius-Mossotti factor as a function of frequency it is possible to estimate the frequency ranges in which a particle will exhibit positive and negative DEP. For Au NPs these ranges will be determined empirically and by modeling.

A useful solution for Re[K(ω)] which illustrates its dependency on the applied frequency is the derivation found by Benguigui and Lin⁴¹:

$$Re [ K ( ω ) ] = \frac{{ɛ}_{p} - {ɛ}_{m}}{{ɛ}_{p} + 2 {ɛ}_{m}} + \frac{3 ( {ɛ}_{m} {σ}_{p} - {ɛ}_{p} {σ}_{m} )}{{{τ}_{M W} ( {σ}_{p} - 2 {σ}_{m} )}^{2} ( 1 + {ω}^{2} {τ}_{M W}^{2} )}$$

(3) where τ_{MW} is the Maxwell-Wagner charge relaxation time given by τ_{MW}=(∈ₚ+2∈ₘ)/(σₚ+2σₘ). This factor accounts for the rate at which free charges distribute themselves along the surface of a sphere.

The Maxwell-Wagner charge relaxation time describes how charges will accumulate on the surface of a suspended particle based on the conductivity and permittivity of the particle and suspending medium. These charges are within the suspended particle and are located at the interface with the suspending medium.

“Tuning” selectivity and optimizing separation. Careful selection of the suspending medium (based on its conductivity) will ensure selectivity between different analytes per the Clausius-Mossotti factor. The conductivity of the medium will be altered by addition of salts. Ions present in an aqueous solution create a double layer surrounding a particle⁵⁰ and will have electrokinetic interactions with the particle. The thickness of the double layer can be estimated using the Debye-Hückel screening length equation [2,15,16]:

$$d = {( \frac{{ɛ}_{m} kT}{8 π {n}^{o} {z}^{2} {e}_{o}^{2}} )}^{\frac{1}{2}}$$

(4) where ∈ₘ is the permittivity of the medium, k is the Boltzmann constant, T is the absolute temperature, n° is the ion concentration in the bulk of the suspending medium, z is the valency of the suspending medium, and eₒ is the charge of an electron. The thickness of the double layer is inversely proportional to the concentration of ions present in the suspending medium. Also, the double layer thickness is inversely related to the valency of the suspending medium, so it is possible to decrease the thickness of the double layer by increasing the valency of the suspending medium, for example going from NaCl, LiCl, or KCl, to CaSO₄ or MgSO₄. The close proximity of the double layer to the surface of a particle will contribute to a particles response to an oscillating electric field through electrokinetic effects^{38,52}. Therefore, it stands to reason that double layer effects on the movement of a dielectric particle will be more pronounced for small particles in a low ionic strength medium.

The DEP force on a particle will be affected by the presence of a double layer, and this effect is enhanced when analyzing submicron particles and macromolecules. This relation can be better understood by close examination of: F_{DEP}=2πr³ Re[K(ω)]∇Eᵣₘₛ²

(5). The DEP force experienced by a particle is related to the cube of the particle radius (eq. 4). With submicron particles, the contribution of double layer thickness on the DEP force will be more profound than for larger particles having similar dielectric properties because of the relative contribution of the ionic double layer^{43,44}. The effects of conductivity of the medium on DEP transport have been studied by several researchers for sub-micron latex spheres^{42,44}, cells^{45,49}, and silica nanoparticles in aqueous solution. Our initial studies with gold nanoparticles indicate that prior findings for other materials are extensible to gold. Double layer effects will be more pronounced for submicron particles that are suspended in low electrolyte solutions. For gold nanoparticles, it should be possible to separate particles based on differences in size by first working with particles of known size at low electrolyte concentrations and empirically determining how the dielectrophoretic force varies over a given frequency range.

Once it is known how these particles will respond in different electrolyte solutions, separations of differently sized nanoparticles will be conducted. The separation will be dependent on the frequency range at which particles of a given size experience either a positive or negative dielectrophoretic force for a given electrolyte concentration, as determined from the empirical data gathered. Particles will be suspended in an appropriate electrolyte solution by application of an AC field at the desired frequency to trap all particles (positive dielectrophoresis) with the exception of one particle size (negative dielectrophoresis). Once the desired particles are trapped, the particles experiencing a negative dielectrophoretic moment will be flushed from the system. Two possibilities for size-based separations of nanoparticles are described below.

If it is found that for a given electrolyte concentration it is possible to sequentially release the trapped particles, based on size, by stepping the frequency of the applied field up or down, the remaining particles can be separated by altering the frequency to selectively induce a negative dielectrophoretic moment in on a group of particles. Once these particles experience negative dielectrophoresis, they will be flushed from the system. This process of stepping the frequency up or down will be repeated until all particles have been flushed from the system.

A second scenario is one in which the electrolyte concentration and/or the frequency of the applied potential will be changed. In this experimental, it will be necessary to sequentially vary the suspending medium by flushing the system while the particles remain trapped at an electrode. Once the system is flushed with the appropriate suspending medium, the frequency can then be altered to release one group of particles. These particles can then be flushed from the system, and the frequency changed to release other particle groups. This process of changing the electrolyte solution and or the frequency of the applied potential will be continued until all of the particles have been separated into their respective size groups.

F. Detectors

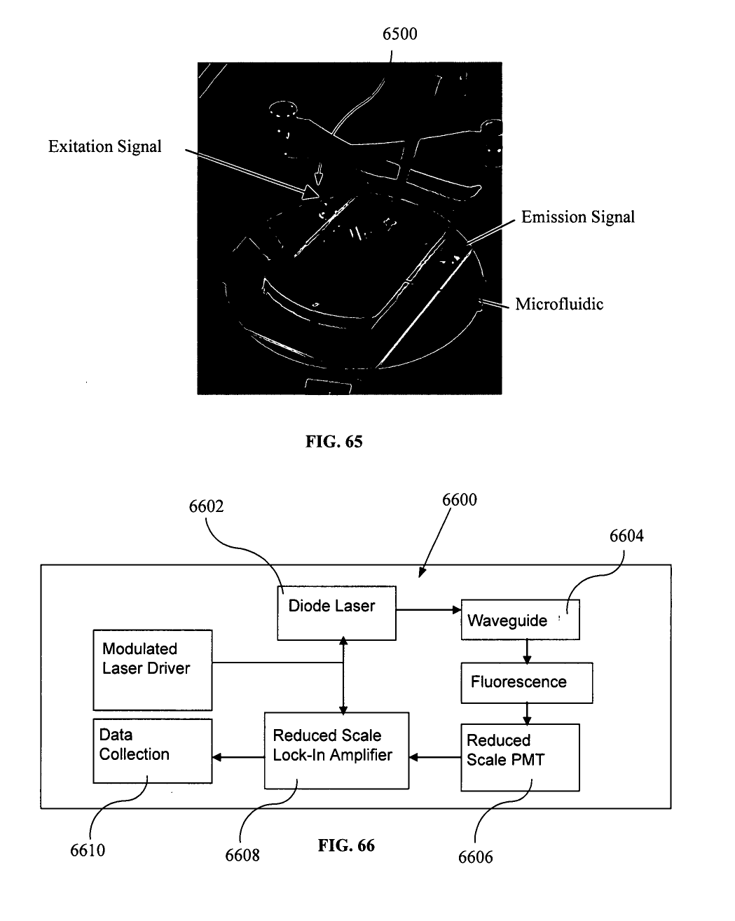

Reagents used for chemical synthesis, products, and by-products preferably will be detectable using a microchemical nanofactory that includes a detection system or detectors. FIG. 65 illustrates a micro-fluidic laser induced fluorescence detection chip 65000 that has been fabricated using a PDMS substrate. The laser dye Rhodamine B was detected using detecctin chip 3000 at 10⁻⁶ M concentration in methanol. The dye solution was injected manually into a 125 μm² fluidic channel. Optical fibers imbedded in the PDMS substrate were used to deliver the excitation light and to collect the emission light. A green laser pointer (Pₒᵤₜ>5 mW, λₘₐₓ=532 nm) served as the source of excitation light. A silicon PIN diode was used to detect the emission signal in conjunction with a lock-in amplifier.

An on-board detection scheme facilitates product purification and sorting; multiple detection points may be useful, such as following each reaction step and each separation step. Wave guides can be fabricated in situ in microfluidic devices by embedding polymeric waveguides into the polycarbonate fluidic layer, coplanar with the fluidic channel and arranged as appropriate for either absorbance (180° arrangement) or fluorescence (90° arrangement, or coaxial) detection. Efforts to date have focused on use of vacuum hot embossed PC chips with SU-8 waveguides, though recent efforts with embedding SU-8 in PC using nanoimprint lithography have met with promising results. This embedded waveguide option is easily incorporated into the fabrication scheme, and occupies minimal space on the device while facilitating simple absorbance, fluorescence, or light-scattering detection at minimal cost. Light sources employed successfully thus far, and planned for the Au nanoparticle microfactory proposed here, include a series of LEDs. Simple, inexpensive photodiodes provide for light detection.

An exemplary embodiment of a detection system 6100 is illustrated in the block diagram of FIG. 66. System 6600 includes a light source 6102, such as a laser, including diode lasers. Light source 6102 will be coupled with waveguide 6104, such as an SU-8 waveguide, at a position on an assembled chip. A light detector 6106, such as a photomultiplier tube, will be placed adjacent to the detection window in the microfluidic channel, and output from the light detector 6106 may be fed to an amplifier 6108. A data collection system 6110, either on chip or off, will be used to receive, store and evaluate data received from amplifier 6108.

### IV. Modular Embodiments of Microchemical Nanofactories

Microchemical nanofactories can be made by using a single unit operation. For example, disclosed embodiments of a process for making dendrimers can be accomplished primarily using a micromixer. However, alternative embodiments may be facilitated by, or may require, using more than one unit operation. Thus, these unit operations can be coupled together. A first method by which a working nanofactory can be made is modular coupling of unit operations. “Modular” coupling refers to, for example, having a first unit operation on a first chip that is then effectively coupled, such as by fluidly coupling, the first unit operation on a first chip to a second unit operation on a second chip. The first chip might be remote in location to the second chip, and the chips may be fluidly connected.

As another example, a first unit operation may be coupled with an off chip macro-type operation, such as purification. Again by way of example, dendrimer synthesis can be accomplished by first mixing reactants on a first nanofactory chip or using a microreactor. The products produced by the nanofactory chip or microreactor are then purified using a conventional purification system, such as an off-chip chromatography process.

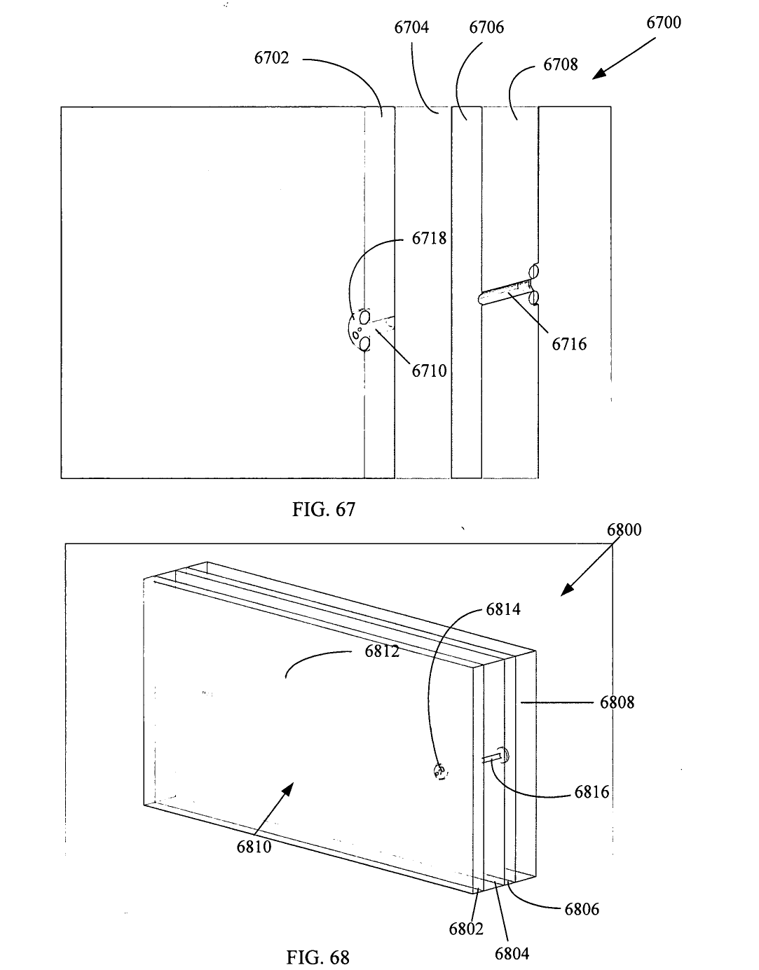

As yet another example, modular unit operations on individual chips can be physically coupled to form an integrated unit comprising plural unit operations. One embodiment of this approach is illustrated in FIG. 67. Nanofactory 6700 has a first unit operation that is performed by a unit device comprising a first lamina 6702 and a second lamina 6704. This first unit device is coupled to a second unit device comprising a first lamina 6706 and a second lamina 6708. The first unit device might perform a function that is the same or different from the function performed by the second unit device. The first unit device and the second unit device are physically coupled in the illustrated embodiment. For example, each of the a lamina 6702, 6704, 6706 and 6708 may have formed therethrough an aperture 6710, 6712, 6714 or 6716. Apertures 6710, 6712, 6714 or 6716 are aligned in the assembled modular device 6700, and are formed to receive a coupler, such as a tie rod or threaded fastener (not shown). A person of ordinary skill in the art will understand couplers or fasteners useful for coupling the modular units together.

Many of the disclosed embodiments are intended for use with fluid systems, either liquid, gas, or combinations of such phases. Thus, the modular unit operations also can include additional sealing features, such as washers or O-rings 6718, 6720. Each of the individual lamina 6702, 6704, 6706 and 6708 may have formed therein a region 6722, 6724 for receiving a seal, such as the illustrated O-rings 6718, 6720.

FIG. 68 illustrates another embodiment 6800 useful for coupling modular unit operations. Nanofactory 6800 has a first unit operation that is performed by a unit device comprising a first lamina 6802 and a second lamina 6804. This first unit device is coupled to a second unit device comprising a first lamina 6806 and a second lamina 6808. For example, the illustrated embodiment includes a serpentine mixer 6810 comprising at least one microchannel 6812, as defined by lamina 6802 and 6804. The first unit device might perform a function that is the same or different from the function performed by the second unit device. The first unit device and the second unit device are physically coupled in the illustrated embodiment. For example, each of the lamina 6802, 6804, 6806 and 6808 may have formed therethrough an aperture (not illustrated) similar to apertures 6710, 6712, 6714 or 6716 for device 6700. These apertures are aligned, and are effective to couple each of the unit modules using ferrules 6814, ¦6816¦_{[bkp3].}

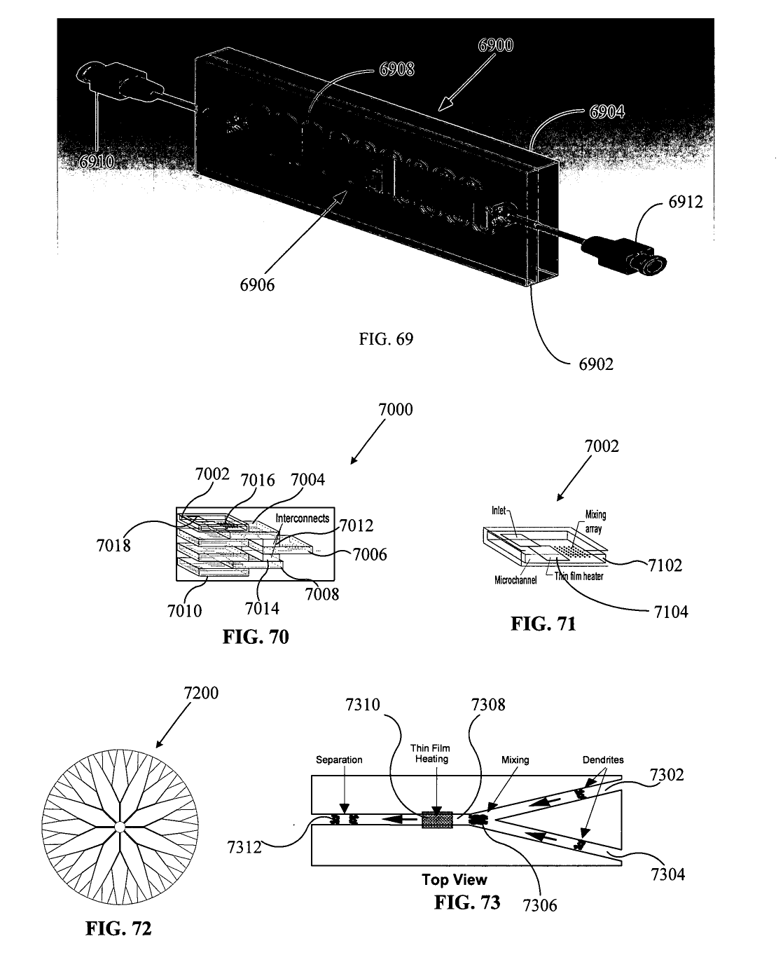

FIG. 69 illustrates another embodiment 6900 useful for coupling modular unit operations. Nanofactory 6900 has a first unit operation that is performed by a unit device comprising a first lamina 6902 and a second lamina 6904. For example, the illustrated embodiment includes a serpentine mixer 6906 comprising at least one microchannel 6908. The first unit device may be coupled to a second and/or third unit device using luers 6910 and/or tube or pipe fittings 6912.

FIGS. 70-71 illustrate a modular approach for both divergent and convergent synthesis. With reference to FIG. 70, nanofactory 7000 includes plural layers, such as may be made by microlamination architecture, 7002, 7004, 7006, 7008 and 7010. Such layers are fluidly coupled by fluid interconnects, such as interconnects 7012 and 7014. Layer 7002 includes a mixing array 7016 and a heater 7018

FIG. 71 is an enlarged view of layer 7002 of device 7000. Layer 7002 includes a microjet mixing section 7102. Thin film heater 7104 (see, for example, Kovacs, G. “Micromachined transducers sourcebook,” McGraw-Hill, 1998) and thermocouples (not shown) can be used to support dendrimer investigation and production. To make thin-film heaters, thin films will be evaporated onto substrates and integrated into microchannels using various bonding techniques.

### V. Integrated Microchemical Nanofactories

The large, fractal sequence of reactions necessary for convergent dendrimer ¦production¦_{[bkp4]} lends itself to the implementation of a fractal nanofactory, or “nanofactory”. One embodiment of such a chemical synthesis factory 7200 is illustrated in FIGS. 72 and 73. The microchemical nanofactory 7200 mimics the geometry of the dendritic molecule it produces. Fractal microchannels have been proposed in heat transfer applications to lower pumping powers and improve thermal distribution on heat transfer surfaces. [See, for example, Chen, Y. and Cheng, P., “Heat transfer and pressure drop in fractal tree-like microchannel nets,” International Journal of Heat and Mass Transfer, 45(13), June 2002, pp 2643-2648; Pence, D. V., “Improved thermal efficiency and temperature uniformity using fractal-like branching channel networks,” Proceedings of the International Conference on Heat Transfer and Transport Phenomena in Microscale, Banff, Canada, 2000, pp. 142-148; Wechsatol, W., Lorente, S., and Bejan, A., “Optimal tree-shaped networks for fluid flow in a disc-shaped body”, Intl J Heat and Mass Transfer, 45(25): 4911-4924, 2002; and Pence, D. V., “Reduced pumping power and wall temperature in microchannel heat sinks with fractal-like branching channel networks,” Microscale Therm Eng, 6(4): 319-330, 2002.] These benefits derive mainly from the minimization of microchannel flow path lengths and the continual disruption of hydrodynamic and thermal boundary layers caused by the regular bifurcation of the flow. The space efficiency of fractal networks is used to improve the channel and unit operation packing density, thereby making the illustrated device compact. Chamber dimensions are on the order of 50 to 100 μm, where dimensions are dictated largely by mixing times, flow rates, residence times, etc.

FIG. 73, an exploded view of one microchannel of a portion of the fractal plate of FIG. 72, schematically illustrates a microchemical nanofactory approach for synthesizing dendrimers. Dendrites flow in microchannels 7302 and 7304 towards a mixing section 7306. The mixed fluid stream then flows through channel 7308 to a heating section 7310 having a heater, such as a thin-film heater. Product, reagents and any byproducts must then be separated in a separation section 7312.

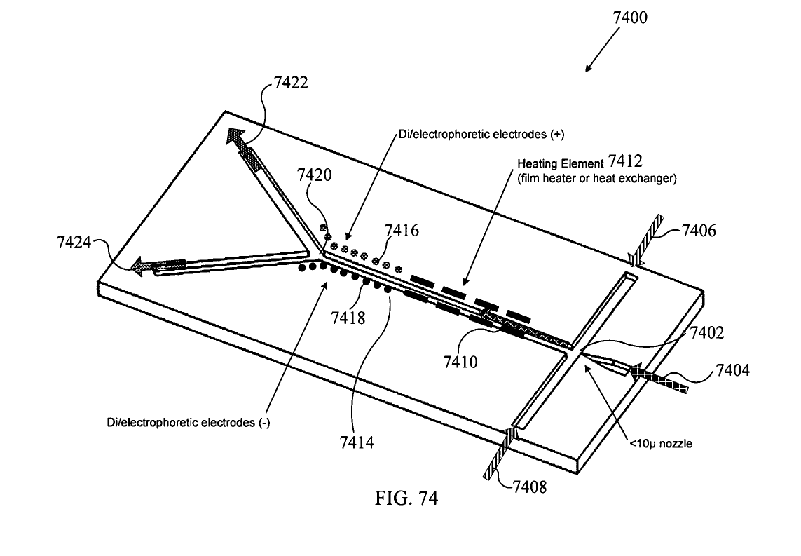

FIG. 74 shows another embodiment of an integrated device 7400 for synthesizing compounds according to the present invention that can be made by microlamination architecture. Although only a single layer is illustrated in FIG. 74, this single layer most likely would be composed of plural, individual laminae registered and bonded to define device 7400.

Device 7400 includes an in-plane nozzle mixer 7402 at a first end there of for introducing at least a first fluid (indicated by arrow 7404) and a second fluid (indicated as arrow 7406) into mixer 7402. A person of ordinary skill in the art will realize that more than two fluid streams can be mixed by mixer 7402, and hence third fluid stream 7408 may be the same as 7404 or 7406, or may be different therefrom, depending upon the compound being synthesized by device 7400. Nozzle mixer 7402 has an orifice of approximately 10 microns.

Once mixed, first fluid stream 7404 and second fluid stream 7406 form a combined fluid stream 7410. Depending upon the chemical synthesis being conducted, combined stream 7410 might involve an endothermic reaction or an exothermic reaction. In such situations, a heating element 7412 might be advantageously positioned downstream of mixer 7402. For example, and in an endothermic reaction, it might be beneficial to increase the fluid temperature rate by heating the fluid stream 7410 to either increase the reaction rate or to provide sufficient thermal energy to heat the reactants sufficiently to overcome any thermal barrier for forming the desired product. Alternatively, if the reaction is exothermic then heating element 7412 might instead be a cooling section, such as a heat exchanger.

As indicated above for the micromixer chemical synthesis of dendrimers, the reaction time can be substantially decreased when mixing occurs using micromixers. Thus, fluid residence time in the reaction portion of the integrated device can be relatively short, on the order of seconds or fractions of seconds. However, in the situation where the residence time might need to be increased so that complete reaction of mixed reagents occurs, fluid residence time can be increased using actuatable valves (not shown for device 7400). Alternatively, integrated devices according to the present invention may use continuous fluid flow, rather than plug flow, in the reaction portion of the device.

Device 7400 includes a separations section 7414 downstream from heat exchange section 7412. For example, where two or more products are formed during the reaction, such products may need to be separated. Alternatively, a product might need to be separated from reagents used to form the product. Thus, the illustrated integrated device 7400 includes separation section 7414. In the illustrated embodiment, the separation section 7414 is a dielectrophoretic separation section; hence integrated device 7400 includes electrodes 7416 of a first polarity and electrodes 7418 of an opposite polarity, with electrode and channel geometries appropriate to yield a non-uniform potential gradient. By creating a potential difference across the fluid channel 7420, materials differing in polarizability can be separated into at least the two illustrated different fluid streams 7422 and 7424. For example, fluid stream 7422 might comprise the desired product, whereas fluid stream 7424 might comprise a recycling stream or a waste stream for might be received for appropriate disposal, depending on the material found in such fluid stream.

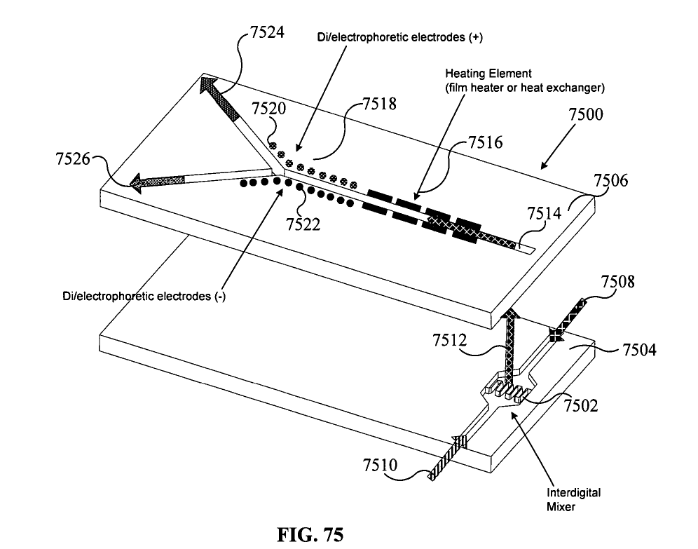

FIG. 75 shows still another embodiment of an integrated device 7500 comprising an out-of-plane, interdigital mixer 7502 with continuous separation of materials in fluid streams. FIG. 75 indicates that device 7500 has two layers 7504, 7506, that together define an integrated device. However, as with the embodiment illustrated in FIG. 74, layers 7504 and 7506 most likely would be made using individual lamina that are assembled by the microlamination architecture methodology described herein. First layer 7504 includes interdigital mixer 7502 at a first end thereof. A first fluid stream 7508 and a second fluid stream 7510 flow to the interdigital mixer 7502, thereby creating a third product fluid stream 7512. Fluid stream 7512 then flows perpendicularly to the flow direction of first fluid stream 7508 and the second fluid stream 7510, and into microchannel 7514 defined by layer 7506.

Device 7500 may include a heating element 7516 positioned downstream of mixer 7502. For example, and in an endothermic reaction, it might be beneficial to increase the fluid temperature rate by heating the fluid stream 7512 to either increase the reaction rate or to provide sufficient thermal energy to heat the reactants sufficiently to be over thermal barrier required for the desired reaction to proceed. Alternatively, if the reaction is exothermic then heating element 7516 might instead comprise a cooling section.

Device 7500 includes a separations section 7518 downstream from heat exchange section 7516. For example, where two or more products are formed during the reaction, such products may need to be separated. Alternatively, a product might need to be separated from reagents used to form the product. Thus, the illustrated integrated device 7500 includes separation section 7518. In the illustrated embodiment, the separation section 7518 is a dielectrophoretic separation section; hence integrated device 7500 includes electrodes 7520 of a first polarity and electrodes 7522 of an opposite polarity, with geometry and configuration appropriate to yield a non-uniform potential gradient across the channel. By creating a potential difference across the fluid channel 7514, materials of different polarizability can be separated into at least the two illustrated different fluid streams 7524 and 7526. For example, fluid stream 7524 might comprise the desired product, whereas fluid stream 7526 might comprise a recycling stream or a waste stream for might be received for appropriate disposal, depending on the material found in such fluid stream.

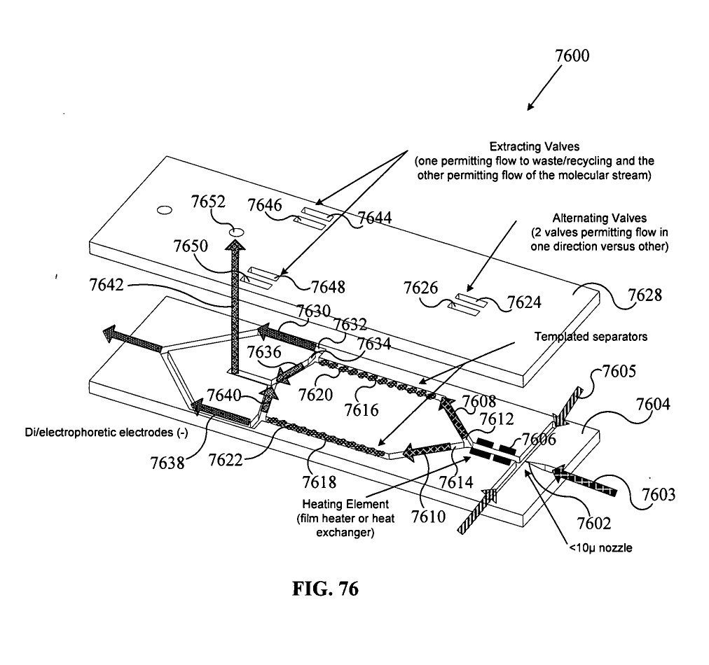

FIG. 76 shows still another embodiment of an integrated microchemical nanofactory 7600. FIG. 76 illustrates in-plane nozzle mixer 7602. As with the prior embodiments, the illustrated embodiments can be made by microlamination architecture using various materials such as stainless steel, polymers, such as polycarbonate, and elastomeric materials, such as polydimethylsiloxane. These materials will be assembled as lamina to provide an overall architecture that, when assembled and registered, as described herein, defines integrated device 7600

Illustrated device 7600 again includes a nozzle mixer 7602 on a first layer 7604. A first fluid feed stream 7603 and a second fluid stream 7605 are mixed using mixer 7602. Additional fluid streams also can be mixed using mixer 7602.

Downstream of mixer 7602, device 7600 includes a heating element 7606, as with the embodiments described above. Downstream of heating element 7606, plural mixed fluid streams 7608 and 7610 exit the heating element 7606 and flow to fluid microchannels 7612, 7614, having separation sections 7616, 7618, respectively. It will be understood by a person of ordinary skill in the art that the number of fluid channels exiting the heating element portion 7606 is not limited to the two fluid channels 7612, 7614 illustrated in FIG. 76. One reason for having plural fluid flows in the illustrated embodiment 7600 is that the separation technique can be different from the substantially continuous dielectrophoretic techniques indicated in previous embodiments. For example, in the separation section of this application templated separators were described that were made, for example, from polymeric materials having pores that were introduced for separation of specific materials one from another. Thus, device 7600 may include a first templated separator 7620 and a second templated separator 7622. Furthermore, separator 7620 might have the same structure as separator 7622, and hence operate identically for purposes of separating materials, or separator 7620 and separator 7622 might be designed to have different sorbents for performing different separations.

Templated- and non-templated sorbent-based separation techniques may function best for non-continuous flow methods, as opposed to continuous flow. For continuous flow through a separation channel having a templated or non-templated separation embodiment, a first fluid portion entering the separation channel would overlap with a second fluid portion entering the fluid channel. This would eradicate any separation that may have occurred by flowing the first fluid portion into the separator.

To guide various batch portions of the mixed fluid stream into the separators 7620, 7622, actuatable valves 7624, 7626 are included in layer 7628. Batch flow through the separation portions 7616, 7618 can be facilitated by effectively actuating valves 7624, 7626 at an appropriate time.

Following the separation sections 7616, 7618, a fluid stream 7630 comprising the desired material is guided down a first fluid channel 7632. A second fluid channel 7634 is provided for a fluid stream 7676 comprising, for example, either recyclable material or waste material that has been separated from the desired material. Because there are plural (two in the illustrated embodiment) separation portions 7616, 7618, the separated fluids from separator 7622 also are bifurcated into a stream 7638 comprising the desired material and a fluid stream 7640 comprising waste or recyclable material. Fluid streams 7676 and 7640 then may combine to form a single waste or recyclable fluid stream 7642, and fluid streams 7630 and 7638 also may combine to form a single product stream.

In order to guide fluid streams to the appropriate fluid channel, device 7600 may include actuatable valves 7644, 7646, 7648 and 7650. A pair of valves selected from valves 7644, 7646, 7648 and 7650 permit fluid flow to the waste or recycling fluid channel, which leads to an outlet pore 7652. The other pair of extraction valve permits flow of the desired fluid stream either to an outlet pore to deliver the desired synthesized compound, or alternatively to a further reaction portion of the integrated microchemical nanofactory 7600 to continue performing additional reactions on the product from the first portion of the device.

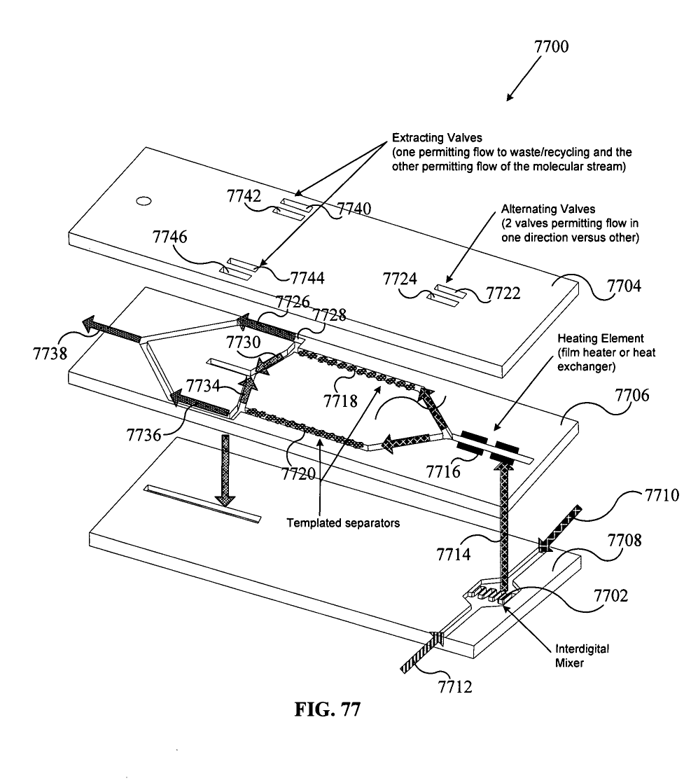

Still another embodiment of an integrated device 7700 is illustrated in FIG. 77. Device 7700 includes an out-of-plane, interdigital mixer 7702. Device 7700 comprises three layers, 7704, 7706 and 7708. A first fluid stream 7710 and a second fluid stream 7712 flow into interdigital mixer 7702 to produce a third fluid stream 7714 that flows perpendicular to the flow of the first stream 7710 and the second stream 7712. The mixed fluid flow stream 7714 then flows into second layer 7706 comprising a heating element 7716.

Separators 7718 and 7720 are provided downstream from the heating element 7716. To guide various batch portions of mixed fluid stream 7714 into the separators 7718, 7720, actuatable valves 7722, 7724 are included in layer 7704. Batch flow through the separators 7718, 7720 can be facilitated by effectively actuating valves 7722, 7724 at an appropriate time.

Following the separators 7718, 7720, a fluid stream 7726 comprising the desired material is guided down a first fluid channel 7728. A second fluid channel 7730 is provided for a fluid stream 7732 comprising, for example, either recyclable material or waste material that has been separated from the desired fluid material. Because there are plural (two in the illustrated embodiment) separators 7718, 7720, the separated fluids from separator 7720 also are bifurcated into a stream 7736 comprising the desired material, and a fluid stream 7734 comprising waste or recyclable material. Fluid streams 7726 and 7736 then combine to form a single fluid stream 7738.

In order to guide fluid streams to the appropriate fluid channel, device 7700 may include actuatable valves 7740, 7742, 7744 and 7746. A pair of valves selected from valves 7740, 7742, 7744 and 7746 permit fluid flow to the waste or recycling fluid channel, which leads to an outlet. The other pair of extraction valve permits flow of the desired fluid stream either to an outlet pore to deliver the desired synthesized compound, or alternatively to a further reaction portion of the integrated microchemical nanofactory 7700 to continue performing additional reactions on the product from the first portion of the device.

### VI. Microchemical Nanofactories Coupled to Front End Processes

Microchemical nanofactories can be coupled with conventional processes, such as front end processes. For example, polymeric materials might be synthesized on site using microchemical nanofactories, and then used as made. This process avoids transportation costs and shelf life issues associated with transporting synthesized polymeric materials to the site of use.

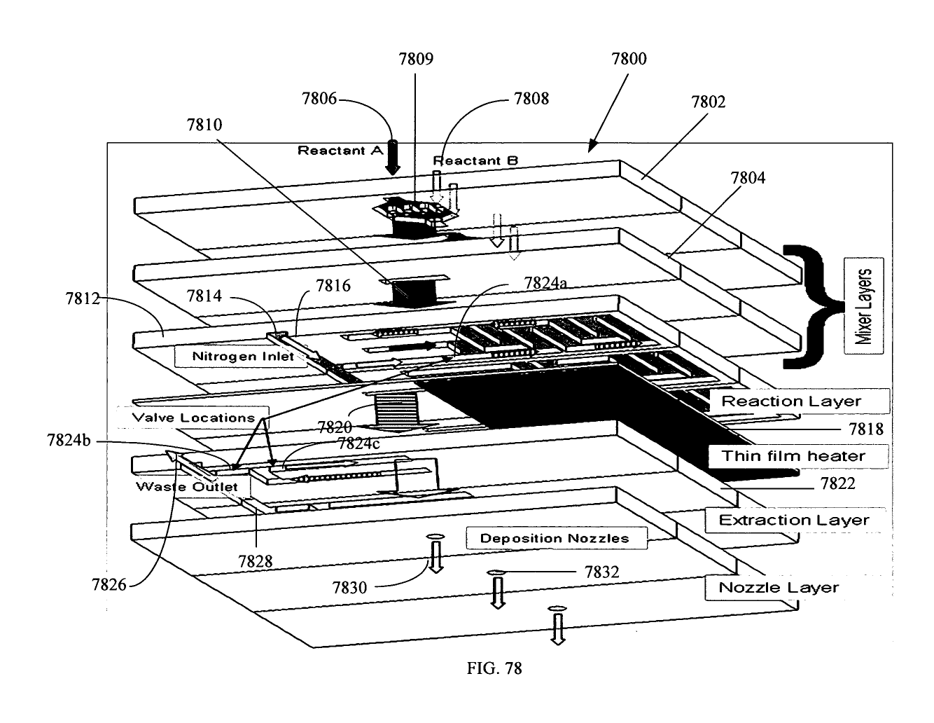

One embodiment of such a system and process is illustrated schematically in FIG. 78. System 7800 illustrates a modular system and subsequent deposition of synthesized materials. System 7800 includes plural mixing layers 7802 and 7804, which can include any embodiments of mixers disclosed herein, or embodiments similar thereto. System 7800 receives a first reactant 7806 and a second reactant 7808 that are effectively mixed using the mixer(s) 7809 provided by mixing layers 7802 and 7804. A mixed fluid layer 7810 then flows into a reaction layer 7812. A person of ordinary skill in the art will appreciate that the embodiment illustrated by FIG. 78 is exemplary only. But, with reference to this specific embodiment, reaction layer 7812 includes a microchannel 7814 for receiving a fluid 7816, such as an inert gas fluid, one example of which is nitrogen. Fluid 7816 might be useful for providing segmented fluid flow, such as with the mixer embodiment of FIG. 23. Certain reactions are facilitated by cooling or heating, and hence the nanofactory 7800 might optionally include a heat transfer section, such as provided by heater 7818.

A product 7820 is produced in the reaction layer 7812. Product 7820 then, if necessary, is provided to a purification or extraction layer, such as extraction layer 7822. Flow between the various layers comprising the nanofactory 7800 can be facilitated by appropriate location of valves, such as valves 7824. Waste material 7826 can be removed from the nanofactory via fluid outlet microchannel 7828.

Microchemical nanofactory 7800 might then be used to deposit product material 7830 onto a desired substrate. How such product material 7830 is applied may depend on the application. Solely by way of example, microchemical nanofactory 7800 includes deposition nozzles 7832 for depositing product material 7830 on a substrate as desired.

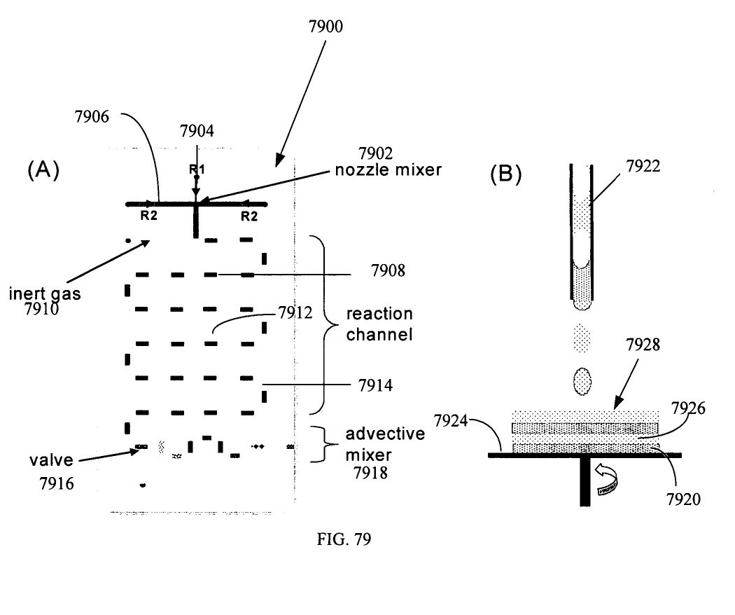

Another embodiment is illustrated in FIG. 79. Microchemical nanofactory 7900 is useful, inter alia, for producing and depositing functional gradient active nanostructures. Microchemical nanofactory 7900 includes a nozzle mixer 7902 for mixing a first reactant 7904 and a second reactant 7906 to produce a product 7908. Product 7908 then is formed into segment flows 7912, such as by using an impinging fluid flow 7910, such as by using an inert gas, one example of which is nitrogen. Segment flows 7912 then enter reaction channel 7914 to produce desired products. Such products then can be applied to a substrate. The deposition of product material can be facilitated by use of a valve, or valves, 7916. Moreover, additional materials may be introduced into a product stream, such a by using advective mixer 7918. This process is further exemplified by the insert, whereby a first product 7920 layer is deposited via deposition nozzle 7922 onto a substrate 7924. In the illustrated embodiment, the substrate is a spinning substrate, which facilitates uniform deposition of product layer 7920. A second product 7926, or the same product but with different functional characteristics, such as particle size, is then deposited onto first product layer 7920. Thus, by depositing such different products 7920, 7926 a functional gradient active nanostructure 7928 can be produced on substrate 7924. Implementation of a nanofactory within a polymer sheet architecture provides the added advantages of an economical “numbering up” through microlamination. The aggregate microsystem can be arrayed to produce an assembly system capable of depositing large volumes of nano-structured materials within hierarchical systems (e.g. patterned films could be deposited onto a conveyorized substrate).

### VII. Specific Implementations

A. Nanostructured Photovoltaics

The search for inexpensive, clean and renewable energy sources has long been a fundamental issue for mankind. Today's major energy source is derived from burning fossil fuels, which are valuable resources in limited supply. In addition, the heat trapping gas from fossil fuel combustion is the largest contributor to the global warming effects. It was estimated that about 10 to 30 TW-year of carbon-free energy will be needed by 2050 to meet the global energy consumption. Among various renewable energy sources, the conversion of sunlight directly into electricity using the photovoltaic properties of suitable materials is an elegant energy conversion process. For PV solar cells to be widely used, the cost must be competitive with conventional energy sources. The key is to develop low-cost manufacturing processes for high efficiency cells.

The maximum thermodynamic limit of conversion efficiency for a single threshold absorber is 31% according to Shockley and Queisser's calculation. This efficiency is attainable from semiconductors with bandgaps from 1.25 to 1.45 eV. The solar spectrum; however, contains photons with energies ranging from 0.5 to 3.5 eV. A major limiting factor, thus, is caused by phonon emissions from the absorbed photons with energy higher than the semiconductor bandgap. One proven successful approach (tandem cells) to go beyond this limit is to use a stack of multiple p-n junctions with cascaded bandgaps tailored to the solar spectrum. Hot carrier, impact ionization, impurity, and multiband¹² solar cells are also potential approaches to exceed this limit. More recently, a variety of novel solar cells are being pursued with a goal to achieve low-cost and high efficiency solar cells. For example, bulk heterojunction devices based on blend of semiconducting polymer with C₆₀ or semiconductor nanocrystals; solid-state dye-sensitized cells based on charge injection from a dye into TiO₂; and nanostructured oxide with semiconducting polymer composite. These novel solar cells have made tremendous improvement in the past few years. Dye sensitized solar cells are currently the most efficient nanostructured solar cells. Central to these devices is a thick films of (10-20 μm) of porous TiO₂ (or wide band gap oxides such as ZnO, or SnO₂) films with adsorbed Dye molecules that are responsible for the absorption of sun light. The porous oxide films are normally fabricated by sintering of oxide nanoparticles and they are responsible for electron collection. The high efficient DSC cells use a liquid electrolyte. The liquid junction provides a good interface for collecting holes and a good pathway for their transport. Thus, efficiency as-high-as 12% could be achieved from a liquid-junction DSC¹⁶. However, liquid-junction is problematic and costly to fabricate and maintain. Efforts have been devoted to develop all solid state DSCs. However, the efficiency of solid-state DSCs is still well below 10%.

On the other hand, thin film polycrystalline I-III-VI Chalcopyrite Cu(In,Ga)(SeS)₂ (CIGSS)-based solar cells have achieved nearly 20% efficiency with a single junction. Its high vacuum manufacturing process makes it too costly. Disclosed embodiments of microchemical nanofactories can be used to manufacture CIGSS-based nanostructured thin film photovoltaics. It is known that the energy bandgap for semiconductor nanocrystals are a function of size. One could maximize the solar absorption by creating a size gradient nanocrystalline semiconductor films for photovoltaics.

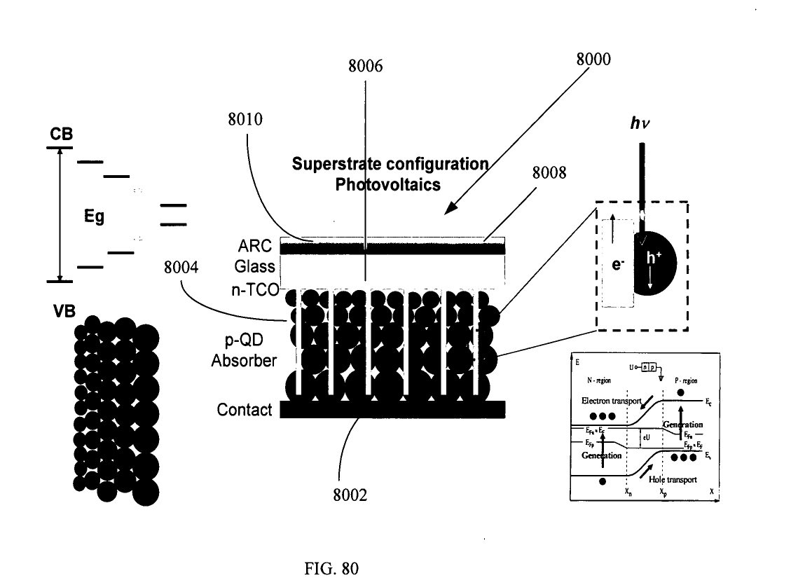

FIG. 80 illustrates one embodiment of a cell structure 8000. Cell structure 8000 includes a substrate layer 8002. For example, the substrate layer 8002 may include substrates made from various materials. Deposited onto substrate 8002 is a p-QD adsorber layer 8004. I-III-VI Chalcopyrite-based p-type quantum dot absorbers with tailored size-gradients will be used for optimum solar spectrum absorption. The high absorption coefficients and a broad range of energy bandgaps from the CIGSS material system make it desirable for PV application. Cell structure 8000 also includes a transparent conducting oxide layer 8006. N-type nanostructured transparent conducting oxide (e.g. ZnO nanowires or nanorods) can be used to form heterojunctions. This design provides two fundamental pathways (increasing photovoltage and photocurrent) to enhance the conversion efficiency. High quality ZnO and GaN semiconducting nanowires can be grown from a vapor phase to form nanostructured transparent conducting oxides.

One aspect of the NPV design involves management of the well-known property of optical interfaces to reflect light which can be modeled using Maxwellian physics as: R=[(n₁−n₂)/(n₁+n₂)]² where R is the Fresnel reflection coefficient and n₁ and n₂ are the indices of refraction of the respective media. As an example, the reflection between ZnO (n=2.04 at 550 nm) and air (n=1.0 at 550 nm) alone is about 11.7%. This reflection can be reduced simply by inserting a film of material between the two original materials that has an index between the two starting media. For instance, placing silica (SiO₂; n=1.46) between the air and ZnO reduces the overall reflection to about 7.5%. This can further be reduced to take advantage of destructive interference if the thickness of the film is made to be quarter wavelength (QW) and the index is optimized to be: nᵢ=\sqrt{n₁·n₂}. Based on this formula, glass/air interfaces (R≈4%) would need indices as low as 1.2 which are difficult to find. Typically MgF₂ films are used with an index of about 1.35 yielding QW air/glass reflectivities around 2% at normal incidence. The quarter-wavelength (QW) effect can be amplified by depositing multiple QW films. Reflectivities as low as 0.5% for air/glass are routinely reported by multi-layer anti-reflective coating (ARC) vendors.

For high wattage applications such as photovoltaics, problems with ARCs include thermal expansion mismatch, thin film processing costs and the inability to coat large, highly contoured or textured surfaces. Processing costs have begun to be addressed through the use of wet deposition methods which are not as precise and therefore do not perform as well (>1.0%) but are significantly less expensive. Other issues include the use of harsh chemical solvents that pose environmental hazards and damage to sensitive optical components. More recently, polymer coatings have been demonstrated with optimized refractive indices using either subwavelength bubbles or nanoparticles. Reflectivities below 0.5% have been reported for these films at certain wavelengths. However, these films provide variable performance across a wide spectrum of wavelengths and are sensitive to incidence angles making them not ideally suited to a “broadband” application such as photovoltaics requiring anti-reflection across a broad spectrum at oblique incidence. Also, these polymer films are not mechanically tenacious particularly for glass surfaces.

Alternatively, if an interface between two media (e.g. air/glass) is made gradual, i.e. a continuous gradient index of refraction is implemented over some finite thickness on the order of a few hundred nanometers, the interface can be made to reflect even less light than QW films. These gradient surfaces can be thought to have a low net reflectance based on the destructive interference of an infinite series of reflections at each incremental change in refractive index. One means for producing this gradient is an array of tapered, subwavelength proturbances as shown in FIG. 3. This structure was first reported based on the electron microscopy of the corneas of nocturnal moths by Bernhard who hypothesized that the resultant index gradients were responsible for the reduced eye reflection at night which the moths needed for camouflage. Subsequently, the term “moth-eye” (See, FIGS. 3 and 4) antireflective surface (ARS) has been adopted as describing a tapered array of subwavelength proturbances.

Applications for moth-eye ARS such as flat panel displays, mobile phones, and personal digital assistants have taken advantage of the “broadband” capabilities of moth-eye structures to work across a wider spectrum of wavelengths and incidence angles. Many different approaches have been used to implement moth-eye structures typically involving some type of lithographic approach (holographic, nanoimprint, self-assembled, etc.) followed by wet or dry etching. Wet etching does not provide adequate feature aspect ratios. Dry etching is expensive.

Microchemical nanofactories can be used to produce size and density gradients for increasing the efficiency of photovoltaic films. These processes provide cost-effective methods for implementing broadband ARS. In the current design, at least three optical interfaces exist (air/glass; glass/ZnO; ZnO/Chalcopyrite) yielding an overall reflectance of about 7%.

Two approaches may be used to deposit gradients of silica nanoparticles on a substrate, such as glass. First, a size-gradient film will be deposited from a microreactor, as shown in FIG. 4, and then sintered. A microreactor can be used to produce monodispered silica nanoparticles, as demonstrated by Jensen et al. The reaction involves base-catalyzed hydrolysis of TEOS (Si (OC₂H₅)₄) followed by condensation to give silica nanoparticles. Asymmetrical pore size can be obtained over the thickness of the film is by continuously changing particle size distribution, which when deposited yields a porosity gradient across the thickness of the film. Particle size will be controlled by adjusting TEOS concentration, catalyst concentration and reaction time and temperature. Further, by controlling the dispersity of a suspension, slight aggregation and hierarchical clusters can contribute to overall porosity increase. Subwavelength particle and pore size can be controlled by sintering. For example, FIG. 6 illustrates a ceria layer that has been formed using a microchemical nanofactory and deposited on a substrate. FIG. 6 includes a 2 μm size bar to establish the typical particle and pore size obtained. One alternative to sintering would be to functionalize the silica nanoparticles with carboxylic acid prior to deposition.

A second approach will involve functionalizing and mixing silica nanoparticles with dendritic polymers (dendrimers) such as PAMAM. A variety of micro- and nanostructured Au nanoparticle/PAMAM dendrimers nanocomposites could be obtained by varying the ratios of carboxylic acid functionalized nanoparticles and PAMAM dendrimers. This approach will be pursues using carboxylic acid-functionalized silica nanoparticles with PAMAM dendrimers. FIG. 5 illustrates a structure made possible by mixing dendrimers and silica nanoparticles to create a density gradient nanostructure. The dendrimers serve as a scaffold for dispersing the silica. PAMAM molecules exhibit effective diameters between 1-10 nm so it is expected that some level of controlled assembly will be required above 5-10 nm. Testing of promising substrates will be using a spectrophotometer having an integrating sphere for assessing the contribution of scattering with traceability to less than 0.5% reflectivity and repeatabilities well below 0.1% reflectivity

B. Inorganic NanoBuilding Blocks

Nanoparticles are solid particulates found on a size scale of 10⁻⁹ meters. A variety of materials including ceramics, semiconductors and metals have been prepared in the form of nanoparticles. There has been significant progress in the synthesis of nanocrystals through solution chemistry that many common materials, such as metals, semiconductors, ceramics, superconductors, and magnetic materials can be prepared from solution. The underlying mechanism of a nanocluster and nanocrystal formation process begins with the collision of reactant molecules, followed by chemical reaction, nucleation, and growth. Sugimoto provided a list of requirements for achieving monodispersed particle distribution. The first requirement is “separation between nucleation and growth.” Crystallization from a supersaturated solution will compromise nucleation and growth simultaneously without careful control of the process. Thus, some of the particles will have been formed in the beginning of the process, whereas other new nucleuses form during the growth process of those earlier formed particles. This will lead to particles with appreciable breadth of size distribution. In order to prevent this, a good crystallization process should be limited to a nucleation burst and followed by a controlled growth process. The third requirement is “inhibition of coagulation.” Once particles are in direct contact, they often adhere to each other and are subject to coagulation. The typical measures to inhibit coagulation are use of a stabilizing medium, such as an electric double layer, a gel network, and dispersants. These requirements provide guidance to engineering a process for production of monodispersed nanocrystals. In summary, burst nucleation, controlled growth and inhibition of coagulation are three for achieving monodispersed nanocrystals.

Burst nucleation: Fast and uniform mixing can be used to create a uniform supersaturation for burst nucleation. Micromixers offer features that cannot be easily achieved by macroscopic devices, such as ultrafast mixing on microscale and integration in complex systems. The second feature, such as easy integration with a micro heat exchanger to achieve fast heat transfer, can be used for precisely controlling reaction temperature during the mixing process (either exothermic or endothermic). In addition, the fast heating feature would provide opportunities for burst nucleation through temperature initiated reaction.

Controlled growth: A second factor for achieving monodispersed nanocrystal production is precise control of the crystal growth condition in the diffusion limited regime and without depleting the reactants thus inducing a “defocusing” phenomenon through Ostwald ripening. This is achieved by adding streams of reactants through additional micromixers (at precise locations along the reaction channel where reactants might have depleted) and precise control of residence time (using segmented flow) and reaction temperature (micro-heat-exchanger).

Inhibition of coagulation: In addition to use stabilizing agents, the laminar flow in the microreaction channel would reduce the possibility of particle collisions and alleviate the problem of nanoparticle growth through coagulation. We have clearly observed this phenomenon in our lab and were able to generate solutions of nanoparticles that were stable for several hours without the addition of surfactants. This opens the door for direct point-of-use nanoparticle production with the need of using surfactants. This is desirable for solar cells.

Microreactors offer several advantages to achieve these requirements for nanoparticle synthesis. For example, CdS, CdSe, SiO₂, Ag, and Au nanoparticles have synthesized from continuous flow microreactors. Continuous flow microreactors allow precise control over processing parameters including temperature, residence time, reactant concentration, mixing efficiency, flow characteristics, and/or the ability to create a gradient for these parameters.

One embodiment of a micro-reaction unit process for meeting these requirements is illustrated schematically in FIG. 79. First the reactants 7904, 7906 will be introduced and mixed through a micromixer 7902. The mixture of reactants 7904, 7904 will be divided into plug flows using a gas bubble, such as from an inert gas source 7910, introduced through an integrated valve (not illustrated). Nanocrystals will grow in a micro-channel with uniform temperature controlled by micro heat exchanger (not illustrated). The length of the reaction microchannel 7914 and flow rate will determine the growth time. Further injection of regents (e.g. surfactants or additional reactants) could be introduced through nozzle mixers (not illustrated) positioned along the reaction channel 7914. This capability provides an opportunity to create a series of micro-plug flow reactors with different concentrations (digital chemistry). This can be used to generate functional gradient nanostructured films by directly integrating the microchemical system with an assembly/deposition reactor. An example of a rotating disk reactor is illustrated in FIG. 79.

C. Nanoparticle Nucleation, Growth, and Aggregation of Au Nanoparticle

The broad utility of gold and other metal nanoparticles in applications such as catalysis, sensing, optical applications, and electronics make their synthesis and production important. For example, size-controlled, monodispersed Au nanoparticles deposited on a substrate, such as a transparent conducting substrate, can be used for ZnO nanowire growth. In addition, for developing microreactor production methods, gold nanoparticles are excellent candidates because their chemistry, characterization and stability are more developed and better understood than other systems. Capillary tube reactors have been used as models of more complex microchannel reactors in order to identify appropriate reaction chemistries for use in microchannel reactors. A series of nanoparticle synthesis reactions in capillary tubing with internal diameters of 150-250 μm are being used to explore nanoparticle formation chemistry. A new synthetic method has been developed for producing from about 1 nm to about 3 nm functionalized gold nanoparticles. Au₁₁ (a 0.8 nm particle) can be used as a seed for particle growth, providing preformed ‘nuclei’ upon which larger particles are grown. In situ monitoring of systematic changes in precursor, growth reagent and passivating ligand concentrations, flow rates, microchannel length, and temperature shall be monitored. For example, particle formation will be monitored in the capillary by on-line UV-vis spectroscopy using low volume (<10 μL) in-line ultra-micro flow cells placed at strategic locations along the capillary. Size-dependent optical signatures and plasmon resonance peaks will be used for multiple point, real-time monitoring of the size of the nanoparticles. The effluent of the capillary will be collected as a batch or in a fraction. The nanoparticles can be stabilized, and their self assembly directed during deposition, by using an appropriate ligand shell, typically thiols, such a alkyl and aromatic thiols. Once particles are in direct contact, they often adhere to each other and tend to coagulate. The typical measures to inhibit coagulation use a stabilizing medium, such as an electric double layer, a gel network, or dispersants, which act as passivation agents. A final stream of stabilizing ligands, either neither, but most likely in solution, will be introduced through the final micromixer to functionalize the mature nanoparticles. Gold nanoparticle syntheses are known and can be implemented in disclosed embodiments of the microchemical nanofactory. For instance, the following example is provided by U.S. patent publication No. 2004-0203074-A1.

NaBH₄ (76 mg, 2.02 mmol) was slowly added to a mixture of AuCl(PPh₃) (1.00 g, 2.02 mmol) in absolute EtOH (55 mL) over 15 minutes. After stirring at room temperature for 2 hours, the mixture was poured into hexanes (1 L) and allowed to precipitate over approximately 20 hours. The resulting brown solid was collected and washed with hexanes (4×15 mL), CH₂Cl₂/hexanes (1:1 v/v 4×15 mL) and CH₂Cl₂/hexanes (3:1, 10 mL). The remaining solid was dissolved in CH₂Cl₂ (15 mL) and filtered a second time to remove a colorless, insoluble powder. Crystallization from CH₂Cl₂/hexanes gave Au₁₁(PPh₃)₈Cl₃ (140 mg, 18% yield) as deep red plates.

D. I-III-VI Chalcopyrite Semiconductors

The luminescent property of semiconductor quantum dots provides an excellent opportunity for direct observation using fluorescence and Raman microscope. Semiconductor nanoparticle concentration will be measured by real time Raman and fluorescence microscopy and spectroscopy. Particle growth kinetics will be studied using the same reactor. Monodispersed nanoparticles (e.g. Au nanoparticle) will be used as seeds for growth experiments. The reacting precursors and the seed nanoparticles will be injected through the microreactor. The output particles will be analyzed on line through quasi-elastic-light-scattering in real time. The nanoparticle solution output will also be collected at different times. Two types of nanoparticles (with and without the core) and their aggregates are expected. The collected core-shell (eg. Au/CuInS₂) and CuInS₂ nanoparticles and their aggregates will be dispersed on transmission electron microscope (TEM) grids and examined under TEM. Core nanoparticles provide a clear contrast and reference for determining particle growth via heterogeneous growth or coagulation by TEM. Both laminar flow and segmented flow in the microreaction channel will be implemented and compared. The homogeneous particle nucleation kinetics will be studied through a similar setup. The microreactor will be modified to a micro-stopped-flow reactor. The stopped-flow method is a primary technique for experimental determination of the rate constant of liquid phase chemical reactions. Nucleation and growth kinetics of nanoparticles will be monitored in-situ by Raman, Photoluminescence (PL), UV-Vis absorption, and quasi-elastic light scattering. The chemistry for the synthesis of I-II_VI semiconductor via solution-chemistry is known in the literature. One embodiment comprises a co-precipitation reaction in solution. For example, CuInS₂ nanoparticles could be synthesized by mixing an aqueous solution of CuCl, InCl₃ and Na₂S in a micromixer. Other Group I, III, VI metal compounds could be used to perform the synthesis, such as metal salts, including by way of example and without limitation Cu(NO₃), CuI, In(NO₃)₃, GaCl₃, Na₂Se. A person of ordinary skill in the art also will appreciate that other solvents, such as lower alkyl alcohols, including methanol and/or ethanol, could be used as well. Another approach is to decompose a metalorganic precursor (e.g. (PPh₃)₂CuIn(SEt)₄) using disclosed embodiments of microchemical nanofactories.

The following examples are provided to exemplify certain features of disclosed embodiments of the present invention. A person of ordinary skill in the art will appreciate that the scope of the invention is not limited to the features exemplified.

### EXAMPLE 1

### Dendrimer Synthesis

Dendrimers are highly-branched molecules with fractal morphologies. Dendrimers consist of a core-unit, branching units, and peripheral end groups. Higher generation dendrimers have close-packed peripheral functional groups and a hollow interior. This unique feature provides dendrimers with the capacity to serve as hosts to encapsulate guests in the interior and to conjugate molecules on the surface. There are two major strategies to synthesize dendrimers: divergent approach and convergent approach. The convergent approach starts from the periphery functional groups and synthesize inward to form higher and higher generations of dendrons. Finally, the dendrons react with a core molecule to generate dendrimer. Dendrimers can be synthesized with great precision, thus, ideally, a certain generation of dendrimer has a single size and molecular weight rather than the broad molecular weight distribution characteristic of linear polymers. Their dendritic architecture has shown great potential for a wide variety of applications including catalysis, sensors, drug delivery, light harvesting, MRI imaging and gene transfer techniques. However, the synthesis of dendrimers is a tedious and time-consuming process, for example, some reaction takes a few days to complete. Therefore the limiting factor on the application of dendrimers is often their cost of production. For dendrimers to realize their full potential, methods must be developed by which the uniformity and efficiency can be closely approximated in the production of macromolecules in nature.

Microreactors enhance mixing and heat transfer due to their short diffusion pathways and large interfacial areas per unit volume (10,000˜50,000 m²/m³). In contrast, conventional reactors have surface area to volume ratios of 100 m²/m^{3[}7]. These two features of microreactor improve yield and selectivity, specifically for mass-transport controlled reactions, highly exothermic or endothermic reactions, and reactions with inherently unstable intermediates. In addition to the benefits mentioned above, another attractive advantage is the ability to “number-up” laboratory-scale reactors by simply arraying the identical microreactors without a need for further process development and parameterization. This numbering-up process has been demonstrated by Clariant, where a chemical was created in the quantity of 80 tons per year within a microchannel format.

Continuous microreactors have been used to synthesize EDA-cored PAMAM dendrimers. A high-yield syntheses of generation G-0.5 PAMAM and generation G0.0 PAMAM have been achieved. Most importantly, the mean residence time for the synthesis in the microreactor is seconds in comparison with days in a conventional batch reactor. Dendrimer synthesis can benefit greatly from implementation in a highly-parallel, process-intensified microsystem format.

To explore the potential benefits of microreactors for dendrimer synthesis, a convergent dendrimers synthesis approach using a continuous flow micromixer has been used. The reactions are illustrated in Scheme 1. The convergent approach used 3,5-bis(4-aminophenoxy)-benzoic acid (compound I) as building blocks, N-methyl-2-pyrrolidinone as solvent, and thionyl chloride was used as an activating agent. 4,4′-oxydianiline served as a core molecule for the synthesis of dendrimers. The synthesis of each generation dendron, except dendron 1, includes two steps: activation by an activating agent, such as thionyl chloride, and coupling with building block 1. The synthesis of each dendrimer consists of two similar steps: activation by an activating agent, such as thionyl chloride, and coupling with core 2. This synthesis strategy has been fulfilled through a conventional flask by Washio, with exception of synthesis of dendrimer G1 and dendrimer G2. However the reactions of each generation of dendron and dendrimer required cooling and inert gas protection, and took about 4-6 hours to complete.

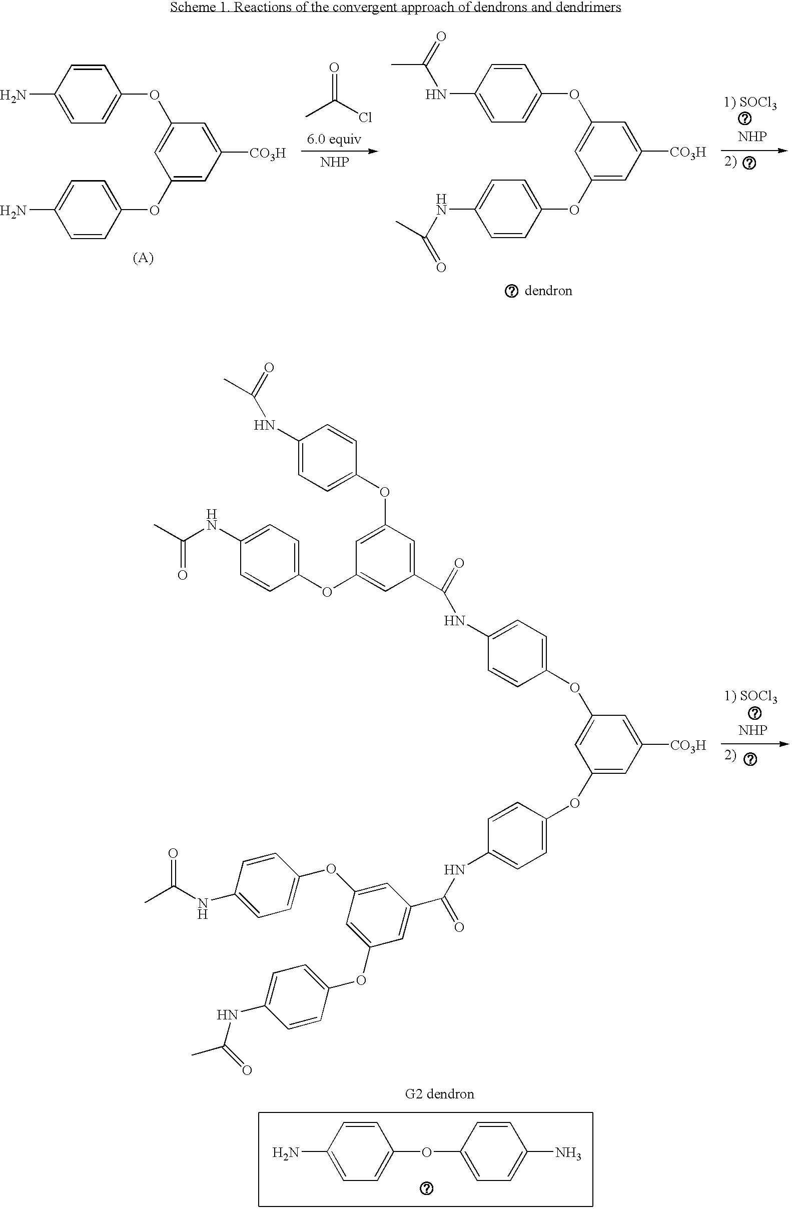

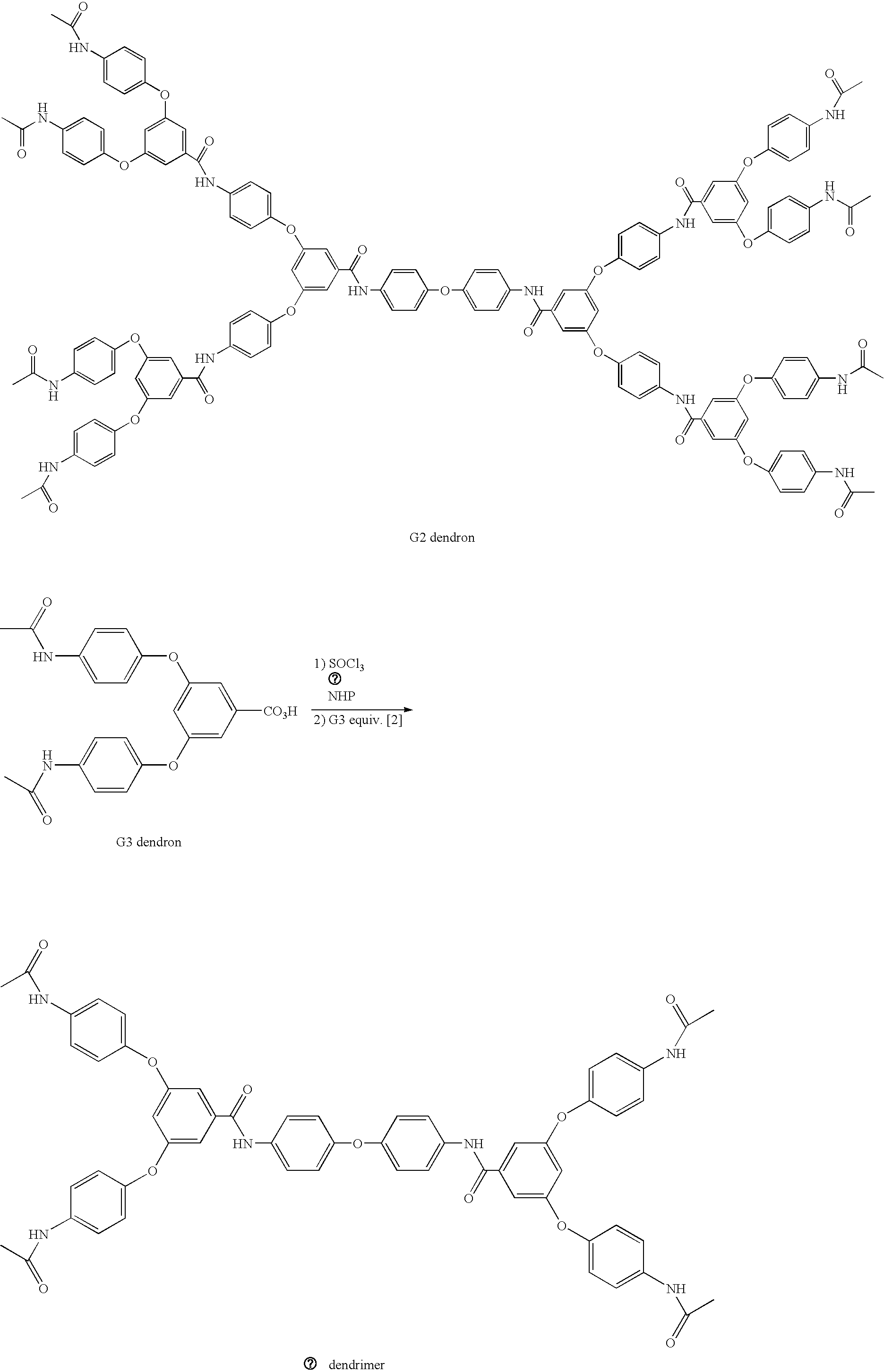

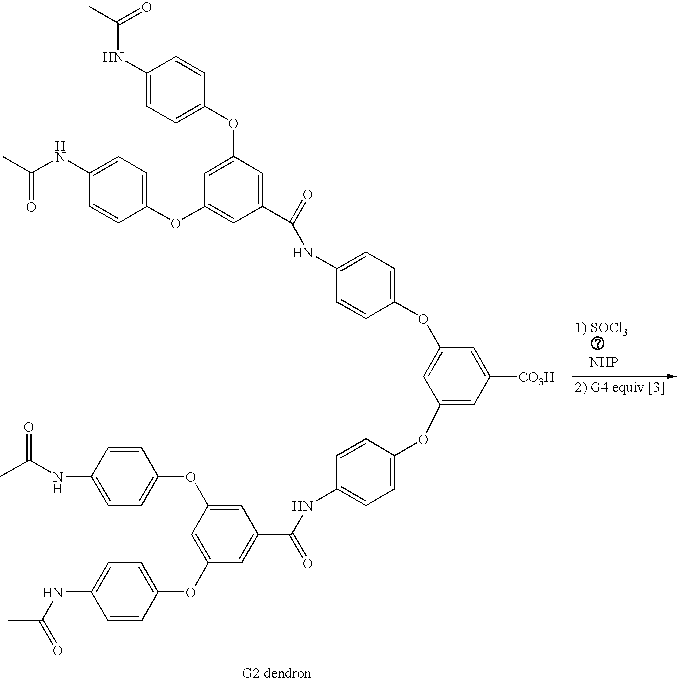

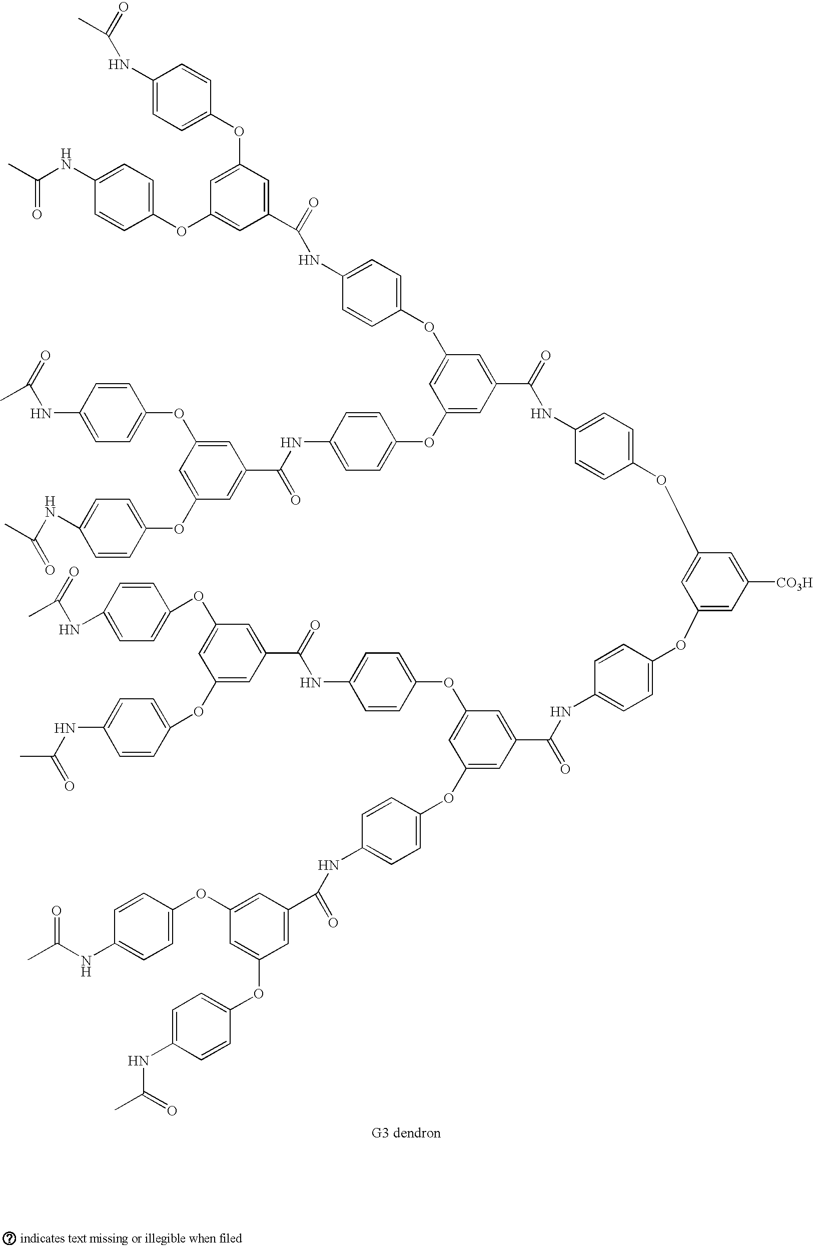

The schematic diagram of the micromixer is given in FIG. 15. The micromixer consists of a mixing element, interdigital microchannels, in the center of the substrate made of thermally grown silicon dioxide. The mixing element is housed within a stainless steel container. Each microchannel has a dimension of 30 μm in width and 100 μm in height. Two streams of reactants were delivered to the interdigital micromixer through two syringe pumps. Each stream was divided into many ultra-thin lamellae by microchannels, and fast diffusion took place as the lamellae left the microchannel chip in the direction perpendicular to the income streams. The ultra-thin lamellae tremendously improved the mass transport between the two reactants, so rapid reaction happened immediately at the outlet of the mixer. FIG. 10 illustrates the principle of mixing via an interdigital micromixer.

B. Dendrimer Synthesis Using Micromixer 602

The construction of an EDA-cored PAMAM includes a series of iterative steps. The first two consecutive steps include: Michael addition of EDA to methyl acrylate followed by amidation of the formed tetraester with EDA. These reactions are illustrated in Scheme 1. Higher generation dendrimers are synthesized following the same procedures either with generation −0.5(G-0.5) or generation 0.0(G0.0).

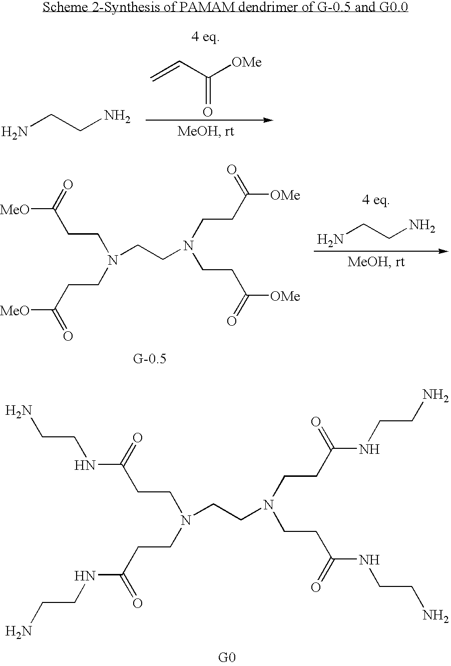

The reactions are exothermic, so coolers and stirrers are used in conventional synthesis of the PAMAM to avoid hot spots, which cause side reactions. The amidation reaction (synthesis of full generations) can form cyclic compounds derived from intra-dendritic cyclization (Scheme 3). These problems inevitably increase the synthetic difficulty and post-separation processes, especially for large-scale synthetic processes. In case of poor mass transfer, the intra-molecular amidation that gives rise to the cyclic product will have more opportunities to occur. To remedy this, a large excess of EDA (50 equivalents) and prolonged reaction times (96 hours) are normally employed for the conventional synthesis of full generation PAMAM.

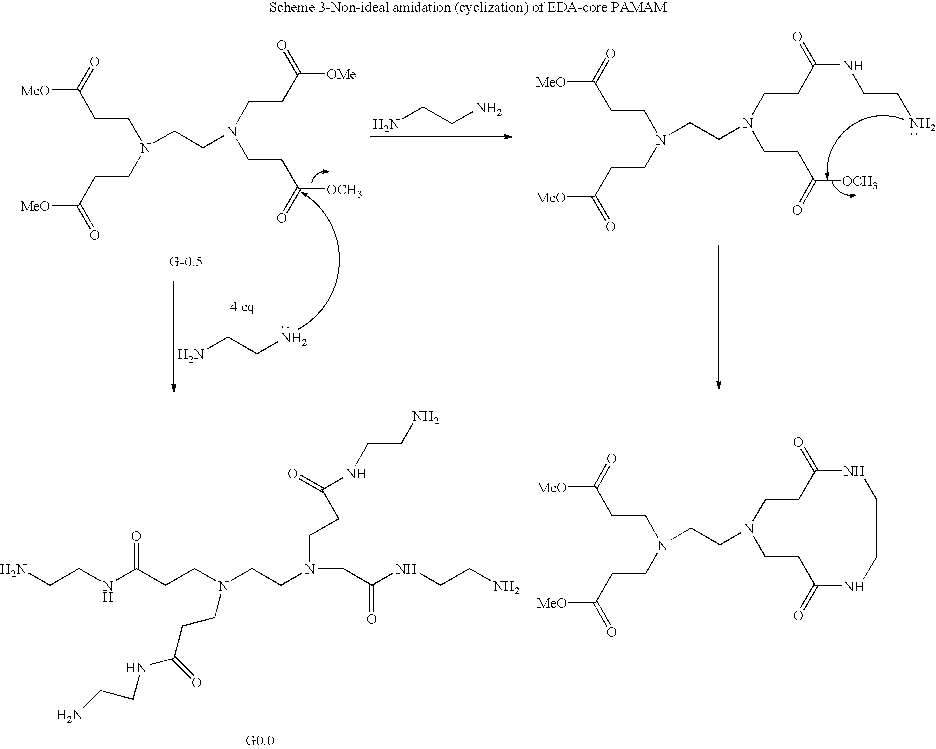

The conventional synthesis of EDA-cored PAMAM has hindered its potential. An economically and time efficient approach to improve the synthetic process will be valuable. Dendrimer synthesis can benefit by highly-paralleled, process-intensified microsystems. Microreaction technology transforms current batch nanoproduction practices into a continuous process with rapid, uniform mixing and precise temperature control. This was demonstrated by using continuous microreactor 602 to synthesize EDA-cored PAMAM dendrimer.

A methanol solution of precursor and a methanol solution of reagent, either EDA (for synthesis of full generations) or methyl acrelate (for synthesis of half generations), were fed into the mixing element of micromixer 1502 through the two micromixer's inlets 1510, 1518, respectively by means of syringe pumps 1506 and 1514 at room temperature. Once the solution streams were introduced into the micromixer 1502, each stream was divided by the micro-scale channels into many thin substreams, which leave the channels perpendicularly to the direction of feed flows and are mixed at the outlet of the micromixer 1502. The mixed solution passes through outlet 1520 of the micromixer 1502 and the tube connected with the outlet with an estimated mean residence time of 1.08 seconds. The adduct solution was collected, solvent was removed by a rotary evaporator, and trace residue of labile reactants was removed under vacuum (0.1 mm Hg, 40° C.).

The first two reactions of EDA-cored PAMAM were conducted using continuous flow microreactor 1502. Starting from the synthesis of the first generation G-0.5 with the starting materials of EDA and methyl acrylate in a solution of methanol, G-0.5 was prepared with 99% yield without observing any side product. Sequentially, generation G-0.0 was synthesized from the starting materials of G-0.5 and an excess amount of EDA with a yield of 98% and without observing any side product.

Dendrimers are difficult to characterize due to their macromolecular size and their highly symmetrical structure. A combination of ¹H NMR spectroscopy and mass spectroscopy were used to determine the structures and purity of the products from the microreactor system 1500 and as produced by conventional batch reactor. NMR spectra were recorded in deuterochloroform, with a 300 MHz Bruker nuclear magnetic resonance spectrometer. Mass spectra were collected using a JEOL MSRoute mass spectrometer in the positive fast-atom bombardment ionization mode.

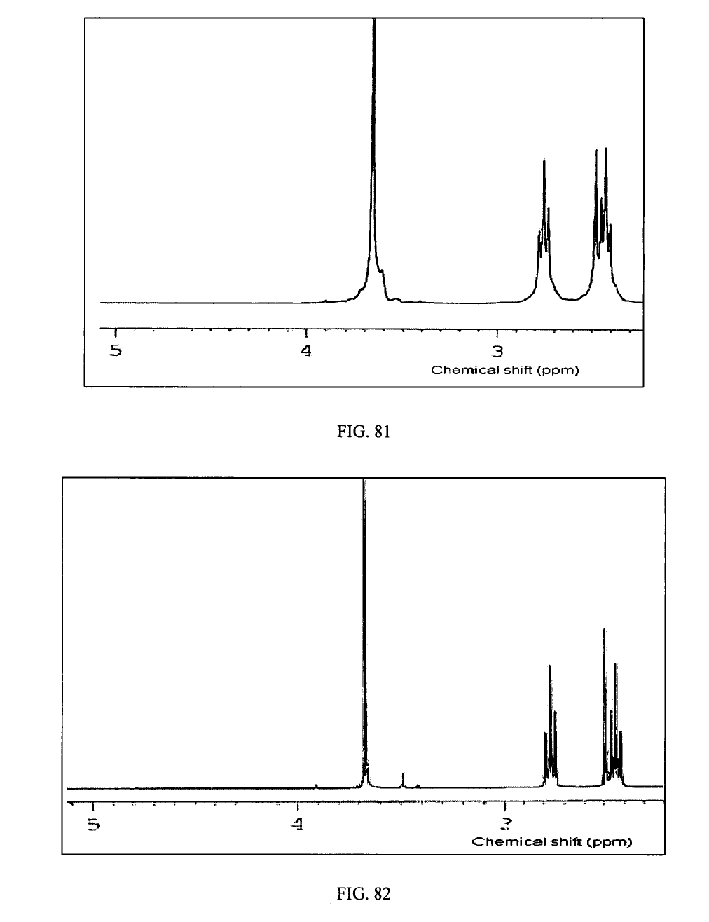

FIGS. 81 and 82 show the NMR spectra of generation G-0.5 synthesized using a conventional approach and in the microreactor system 1500, respectively. The NMR results indicated no side products were produced using microreactor system 1500. On the other hand, the NMR spectrum from the batch reactor shows some side products (shoulders appeared at peak 3.67 ppm in FIG. 81) even after 3 days of vigorous mixing. The side reaction could be caused by intra-dendrimeric cyclization. The detailed NMR spectral characterization is described as follows: the molecular formula of G-0.5 is (^{a}CH₂^{a}CH₂)[N(^{b}CH₂^{c}CH₂^{d}CO₂^{e}CH₃)₂]₂. In the NMR spectra of G-0.5 displayed in FIG. 7 and FIG. 8, the single peak at 3.67 ppm is assigned to proton ‘e’; the triplet peak centered at 2.76 to proton ‘b’; the single peak at 2.49 ppm to proton ‘a’; the triplet peak centered at 2.44 to proton ‘c’.

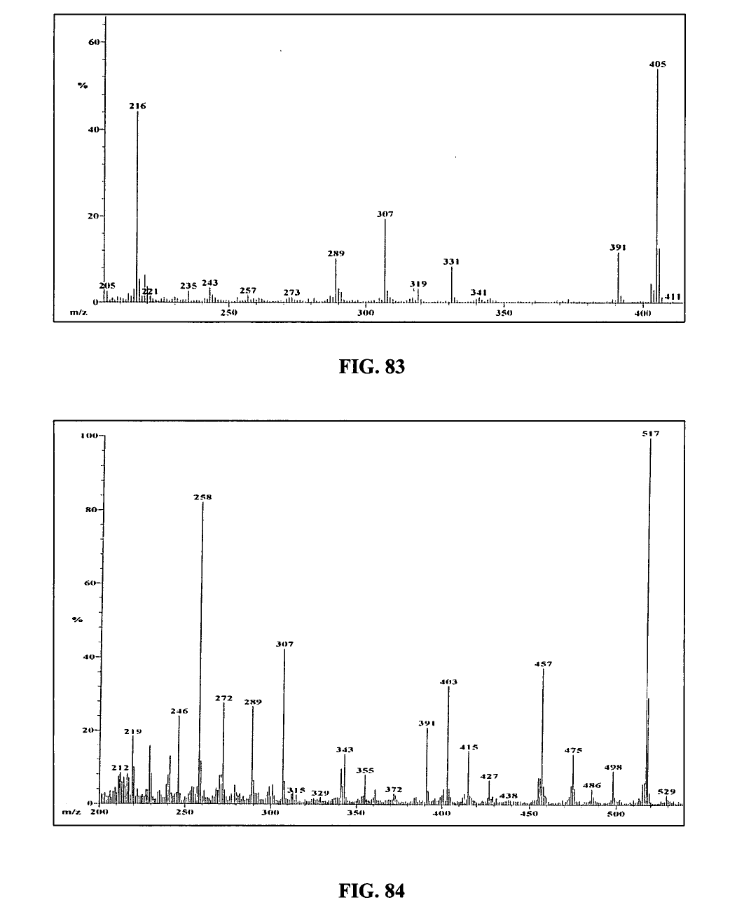

Mass spectra of G-0.5 (molecular weight of 404) and G0.0 (molecular weight of 516) synthesized in a microreactor are shown in FIGS. 82 and 83, respectively. The strong peaks at m/z 405 (M+H) and m/z 517 (M+H) indicate the successful microreactor synthesis of G-0.5 and G0.0 products.

Microreactor system 1500 demonstrated several advantages over the conventional batch process. The conventional reaction requires cooling and stirring systems, dropwise addition of reagents at the beginning to conduct the released heat, and inert gas protection. In contrast, all reactions conducted using micromixer system 600 were continuous flow at ambient temperature without using an inert atmosphere. Moreover, plural syntheses of EDA-cored PAMAM dendrimers using micromixer 1502 showed good reproducibility. The most attractive advantage is that the residence time for micromixer 1502 is 1 second versus 72 hours (for half generation product) and 96 hours (for full generation product) in a conventional batch reaction. In addition, by achieving the high purity of the low generations PAMAMs, further syntheses for higher generation products are considerably eased.

General Methods:

All chemicals, except thionyl chloride (from Sigma Aldrich), were purchased from TCI America, and were used as received. The interdigital micromixer was purchased from Institut für Mikrotechnik Mainz, Germany. A NMP solution of precursor and a NMP solution of reagent, thionyl chloride (for activation of precursor), building block (coupling reaction to synthesize dendrons) or core molecule (to synthesize dendrimers), were fed into the mixing element through the two micromixer inlets, respectively, by means of syringe pumps. Once the solution streams were introduced into the micromixer, each stream was divided by micro-scale channels into many thin substreams, which left the channels perpendicular to the direction of feed flows. In this manner, the feed flows were rapidly mixed by diffusion at the outlet of the micromixer. The mixed solution passed through the outlet of the micromixer at a flow rate of 0.052 cm³/s and the tube (35 cm long and 0.75 mm ID) connected with the outlet with an estimated mean residence time of 3 seconds. Subsequently, the adduct solution was collected, and poured into water, and the precipitate was collected and dried.

Synthesis of Dendron G1:

Solutions of 3,5-bis(4-aminophenoxy)-benzoic acid (building block) dissolved in NMP (concentration of 0.58 mol/L) and 3 equivalent of acetyl chloride dissolved in NMP (concentration of 1.74 mol/L) were introduced into microchannels by syringe pumps at flowrate of 0.026 ml/s respectively. The mixture was collected at outlet and precipitated in water. The precipitate was collected and dried at 120° C.

Synthesis of Dendron G2:

Solutions of dendron G1 dissolved in NMP (concentration of 0.323 mol/L) and 1.04 equivalent of thionyl chloride dissolved in NMP (concentration of 0.336 mol/L) were introduced into microchannels by syringe pumps at flowrate of 0.026 ml/s respectively. The intermediate was collected at outlet. Then solutions of the intermediate and 0.48 equivalent of building block which has been dissolved in NMP (concentration of 0.078 mol/L) were fed into micromixer by syringe pumps at flowrate of 0.026 ml/s respectively. The mixture was collected at outlet and precipitated in water. The precipitate was collected and dried at 120° C.

In order to compare the synthesis results between the micromixer approach and the conventional batch approach, the batch approach was conducted. To a 1.6 ml of solution of dendron G1 (0.517 mmol) dissolved in NMP, 1.04 equivalent of thionyl chloride was added at 0° C. under nitrogen and stirred for 20 minutes at 0° C. and for another 20 minutes at room temperature. Sequentially, 0.48 equivalent of building block 1 (0.248 mmol) was added to the solution and the reaction was taken place for overnight. The mixture was precipitated in water. The precipitate was collected and dried at 120° C.

Synthesis of Dendron G3:

Solutions of dendron G2 (0.0613 mmol) dissolved in NMP (concentration of 0.153 mol/L) and 1.1 equivalent of thionyl chloride (0.0675 mmol) dissolved in NMP (concentration of 0.169 mol/L) were introduced into microchannels by syringe pumps at flowrate of 0.026 ml/s respectively. The intermediate was collected at outlet. Then solutions of the intermediate and 0.48 equivalent of building block which has been dissolved in NMP (concentration of 0.0368 mol/L) were fed into the micromixer by syringe pumps at flowrate of 0.026 ml/s respectively. The mixture was collected at outlet and precipitated in water. The precipitate was collected and dried at 120° C.

Synthesis of Dendrimer G1:

Solutions of dendron G1 (0.2 mmol) dissolved in NMP (concentration of 0.2 mol/L) and 1.5 equivalent of thionyl chloride dissolved in NMP (concentration of 0.3 mol/L) were introduced into microchannels by syringe pumps at a flowrate of 0.026 ml/s respectively. The intermediate was collected at outlet. Then solutions of the intermediate and 0.5 equivalent of core molecule which has been dissolved in NMP (concentration of 0.05 mol/L) were fed into micromixer by syringe pumps at flowrate of 0.026 ml/s, respectively. The mixture was collected at outlet and precipitated in water. The precipitate was collected and dried at 120° C.

Synthesis of Dendrimer G2:

Solutions of dendron G2 (0.0885 mmol) dissolved in NMP (concentration of 0.0885 mol/L) and 1.5 equivalent of thionyl chloride (0.1328 mmol) dissolved in NMP (concentration of 0.1328 mol/L) were introduced into microchannels by syringe pumps at flowrate of 0.026 ml/s respectively. The intermediate was collected at outlet. Then solutions of the intermediate and 0.5 equivalent of core molecule which has been dissolved in NMP were fed into micromixer by syringe pumps at flowrate of 0.026 ml/s respectively. The mixture was collected at outlet and precipitated in water. The precipitate was collected and dried at 120° C.

Measurement:

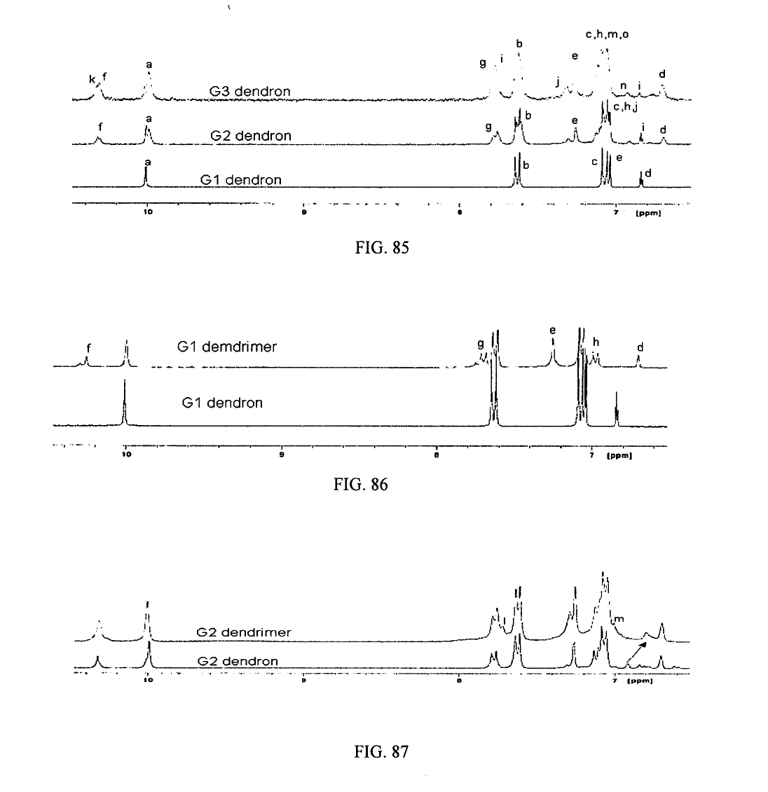

A combination of ¹H NMR spectroscopy and mass spectrometry has been used to determine the purity and the structures of the products from the micromixer and from the conventional batch reactor. Syntheses from micromixer provided comparatively pure product without measurable side product. On the other hand, the NMR spectrum of G2 dendron synthesized from the batch reactor shows some side products even after 20 hours of vigorous mixing. The ¹H NMR spectra of dendrons G1, G2 and G3 synthesized via micromixer are shown in FIG. 84. 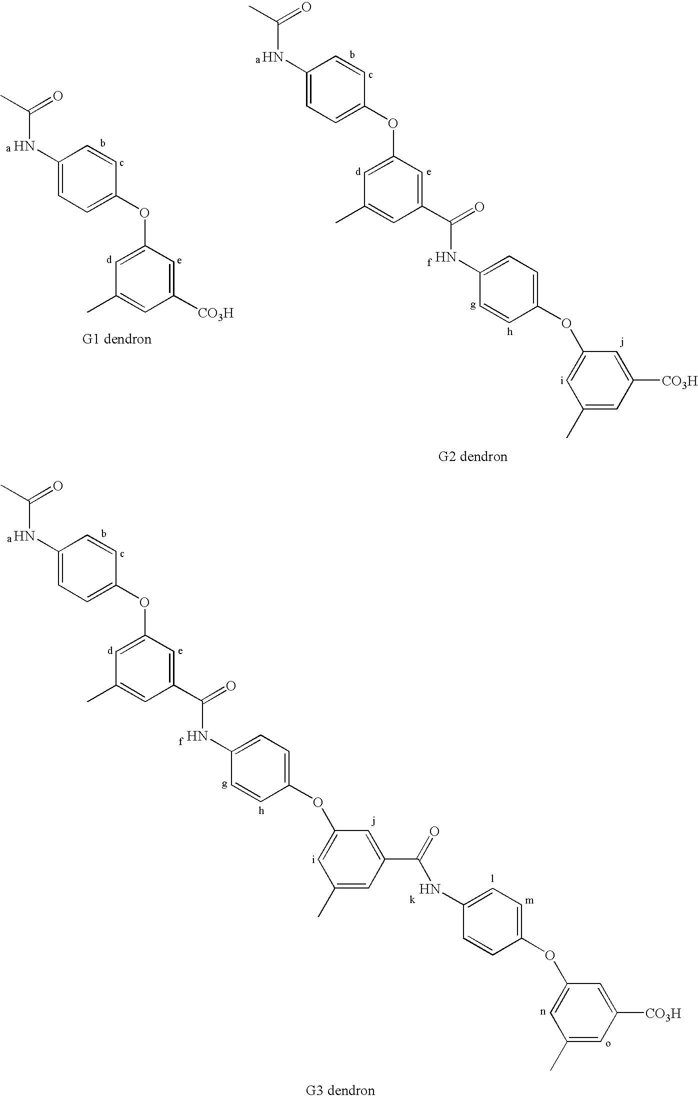
The ¹H-NMR spectra of G1 dendrimer is compared with G1 dendron shown in FIG. 85. The signals assigned to the protons of position d shift from 6.84 to 6.71 ppm, position e from 7.04 to 7.25 ppm and the signals in 10.26 (f), 7.71 (g) and 6.98 (h) appear after the coupling reaction, indicating the formation of G1 dendrimer. 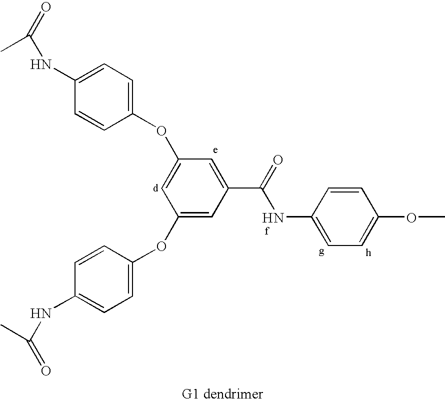
The ¹H-NMR spectra of G2 dendrimer is compared with G2 dendron shown in FIG. 86. The signals assigned to the protons of position i shift from 6.85 to 6.80 ppm, and the signals in 7.73 (l) and 7.02 (m) appear after the coupling reaction, indicating the formation of the G2 dendrimer.

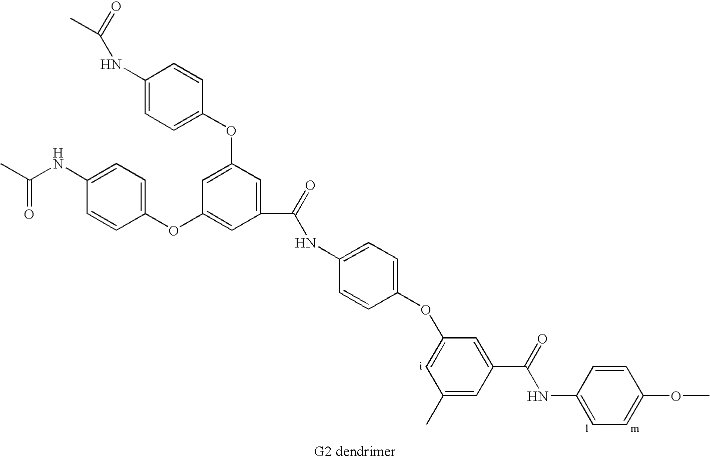

The micromixer demonstrates several advantages over the conventional batch process. The conventional reaction required cooling and stirring, inert gas protection. All reactions conducted using the micromixer were performed continuously at ambient temperature without cooling, stirring and the need for an inert atmosphere. A number of experiments were conducted using the micromixer and the results demonstrated good reproducibility. The most attractive advantage of the micromixer approach is that the residence time is 3 seconds versus several hours in a conventional batch reaction. Rapid, continuous flow, high yield and selectivity, and most importantly, a facility for numbering-up the process for industrial production scale, microreactor based synthesis appears to be a promising approach for dendrimer synthesis.

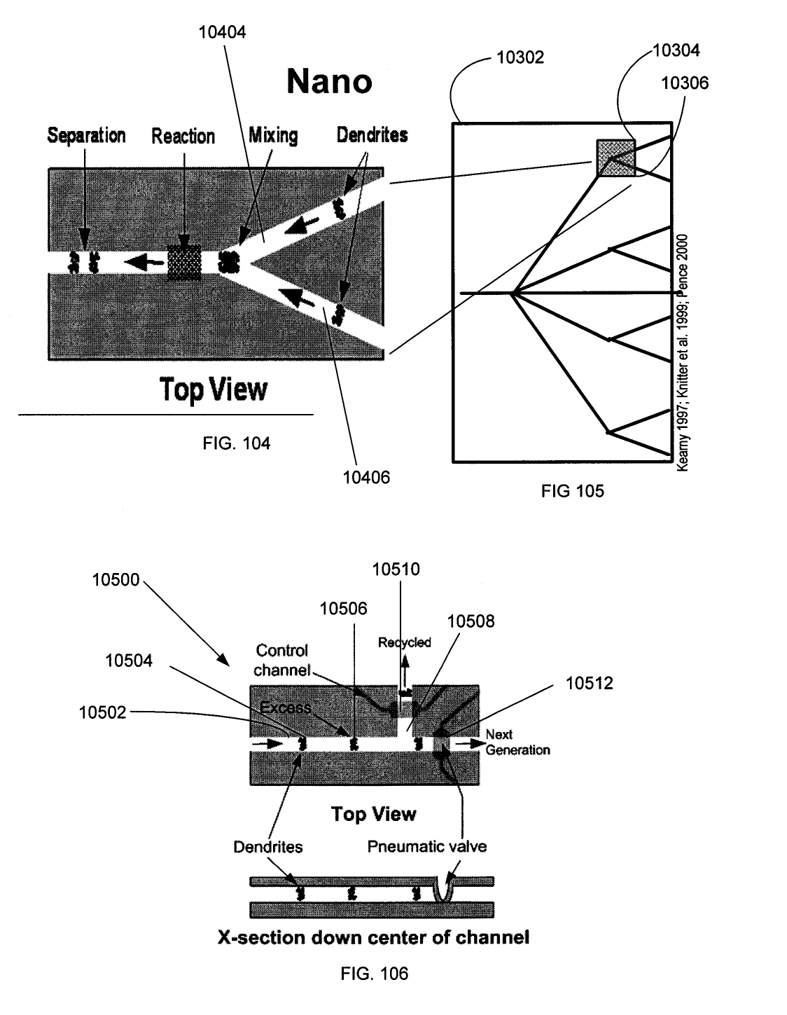

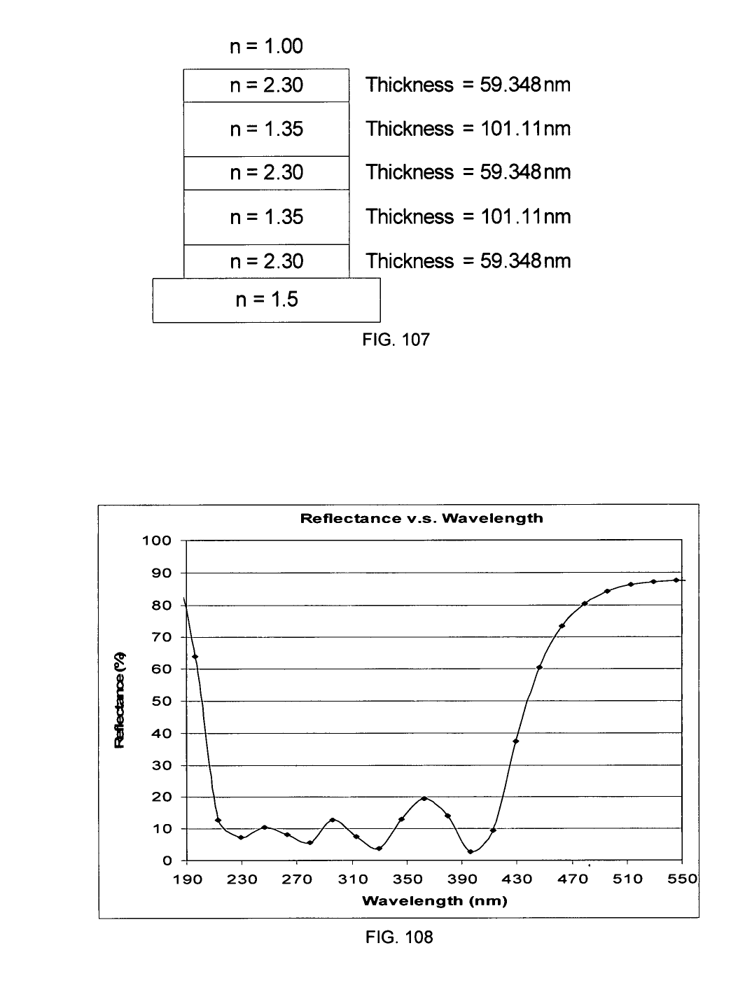

A person of ordinary skill in the art also will appreciate that other nanofactory architectures also can be used to make desired compounds. For example, and with reference to the synthesis of dendrimers, a nanofactory comprising a linear fractal plate can be used to synthesize dendrimers. One example of such a nanofactory is illustrated in FIGS. 103-105. Fractal plate 10002 includes plural microchannels, exemplified by microchannels 10004 and 10006. A plan view blow up of a portion of the fractal plate 10002 is shown in FIG. 104. A cross sectional schematic view is shown in FIG. 105. FIG. 105 illustrates that dendrites 10504 and excess material 10506 move through microchannel 10502. Excess material can be removed via a second microchannel 10508. This embodiment of a nanofactory 10500 includes at least one valve 10510 operatively associated with microchannel 10508 for controlling removal of excess material. Likewise, at least a second valve 10512 is operatively associated with valve 10502 for controlling synthesis of dendrimers as product continues to flow down microchannel 10502.

### EXAMPLE 2

In recent years, transparent conducting oxides (TCO) have increasingly drawn people's attraction due to their wide bandgap properties for optical and electrical applications. Transparent zinc oxide thin film with a bandgap of 3.2˜3.4 eV is a n-type semiconductor. Many studies of ZnO thin films have been reported and applied in various areas such as gas sensors, transparent electrodes in photovoltaic solar cells, and transparent thin film transistors. ZnO thin films have been prepared by many different techniques including radio frequency magnetron sputtering, evaporation, metal organic chemical vapor deposition (MOCVD), electrochemical deposition, electroless deposition, spray pyrolysis, and chemical solution deposition.

Among them, chemical solution deposition, also called chemical bath deposition (CBD), has many significant advantages as a result of low cost and low temperature processing nature. Continuous flow microreactors introduce a constant flux of reactant solution to the substrate. This continuous process allows a precise control over the homogeneous reaction of the chemical bath solution before it impinges on the substrate. A particle-free reactant flux is generated by using a short residence time. Using this particle-free flux, molecule-by-molecule heterogeneous growth mechanism have been promoted that prevents particle-by-particle growth. A microreactor operating in a particle formation regime was used to deposit transparent ZnO thin films. This technique, refer to as Chemical Nanoparticle Deposition, follows a thin film growth mechanism based on nanoparticle formation and sticking. The resulting film is highly transparent nanocrystalline ZnO with a hexagonal structure. A functional ZnO MISFET with an effective mobility of 0.16 cm²/Vs. and current on-to-off ratio of ˜10⁴ was successfully fabricated using this technique.

FIG. 87 illustrates one embodiment of a deposition system 8700 consisting of a microprocessor controlled peristaltic pump (Ismatec REGLO Digital), three 1.22 mm ID Tygon ST tubings (Upchurch Scientific) 8702, a T-mixer (Upchurch Scientific) 8704, a 3″ diameter stainless steel metallic plate 8706, and a 2″ diameter×0.75″ thick heating hotplate 8708 with a temperature controller (Watlow) 8710. A reactant stream A and B were initially pumped into Tygon tubing 8712 individually at a flow rate of 27 ml/minute and allowed to mix through the T-mixer 8704. Stream A comprised 200 ml 0.005 M zinc acetate and 10 ml 0.25 M ammonium acetate. Stream B comprised 200 ml 0.1 M sodium hydroxide. The resulting mixture, from the T-mixer 8704, then was passed through a ˜1 m long coil 8714 and kept immersed in a hot water bath 8716 maintained at 80° C. (using a VWR hot plate stirrer).

The oxidized silicon substrates measuring 10×15 mm were initially sonicated in an ultrasonic bath containing 1 M NaOH solution for 20˜30 min and then cleaned according to a standard AMD (Acetone, Methanol, and De-Ionized Water) procedure. Finally, the cleaned substrates were dried under a stream of nitrogen gas before being used for deposition. The substrate 8718 was taped to the 3″ diameter stainless steel metallic plate 8706 and heated on the metal hotplate 8708 at 80° C. Once the process was completed, the substrate 8718 was removed from the plate 8706, washed with DI water and dried under a stream of nitrogen gas.

Transmission Electron Microscopy (TEM) sample was obtained by dipping a copper grid (with thin lacey carbon film) in the hot solution, collected from the deposition system, for about 10 seconds. Scanning Electron Microscopy (SEM) was employed to study the surface morphology and microstructure of the obtained films on oxidized silicon substrates. X-ray Energy Dispersive Spectrometer (EDS) was used to evaluate the chemical composition of the thin films. X-ray Diffraction (XRD) (Siemens D-5000) with Cu Kα radiation was performed to determine the phase and crystalline orientation of the deposited thin films. The optical absorption and transmission analysis of the ZnO thin films were measured by a UV-Vis Spectrophotometer (Ocean Optics Inc, USB 2000 optic spectrometer) for both optical bandgap estimation and transmittance measurement.

For ZnO MISFET fabrication, a heavily boron (p+) doped silicon substrate served as the gate in an inverted-gate structure. Silicon dioxide with a thickness of 100 nm was thermally grown on top of the silicon substrate and a 500 nm gold layer for gate contact was sputtered on the backside of the Si substrate. ZnO thin films were deposited on top of the SiO₂ layer using the system 8700. After the deposition, the substrate 8718 was removed from the metallic plate 8706 and a post annealing process was performed at 600° C. for 30 minutes in an air furnace. The 300 nm aluminum source and drain contacts were then evaporated on top of the ZnO layer through a shadow mask with a channel width-to-length ratio of 12 to complete the process of fabricating ZnO Metal-Insulator-Semiconductor Field Effect Transistors (MISFETs).

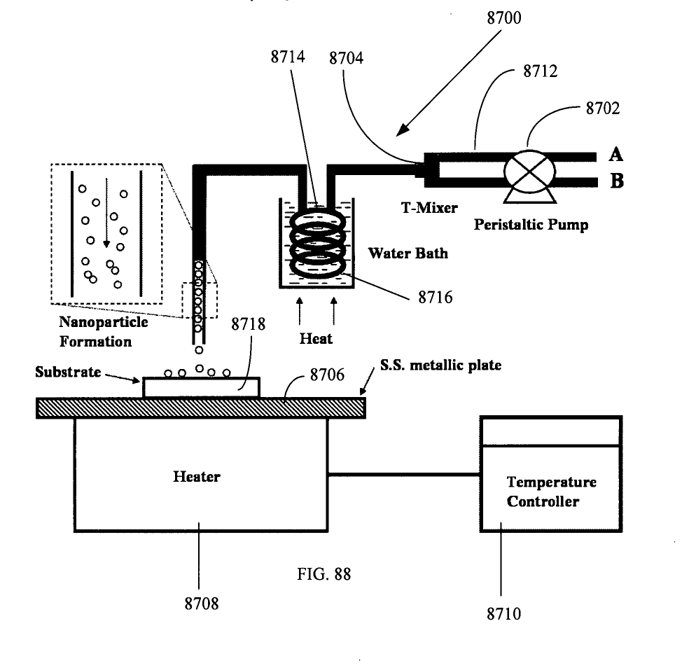

The TEM micrograph (FIG. 88) shows several tens of round shape nano-sized particles collected from the solution (some rod shape nanoparticles could also be observed). High resolution TEM images establish that these nanoparticles are crystalline. TEM electron diffraction characterization further confirmed the polycrystalline structure of the as deposited ZnO thin films (JCPDS-ICDD card No. 79-2205). The TEM results indicate the occurrence of homogeneous particle formation in the microchannel.

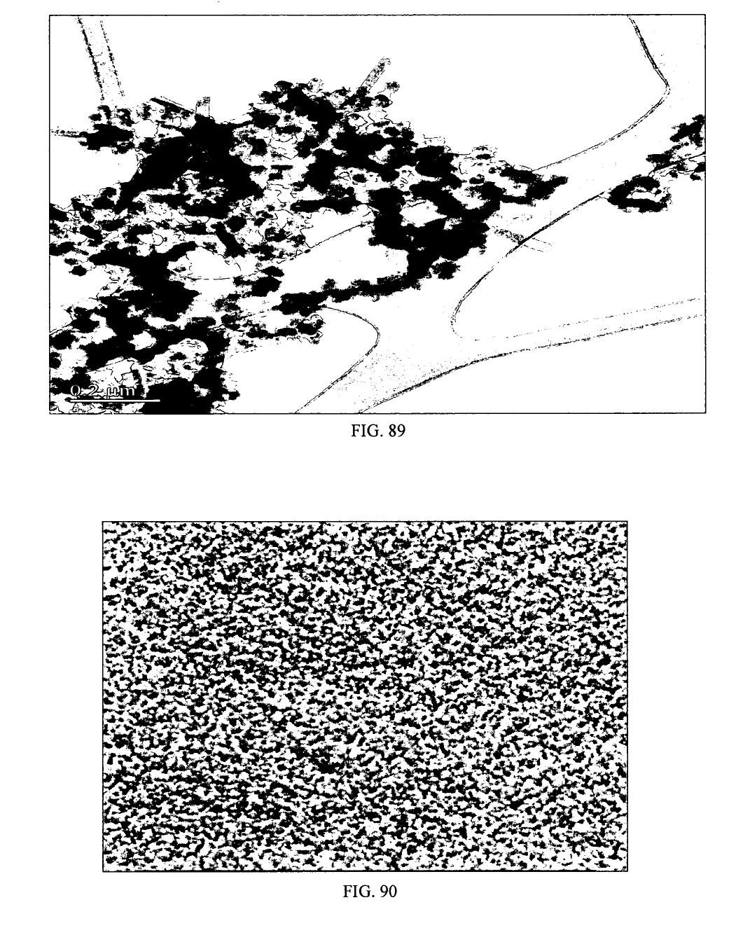

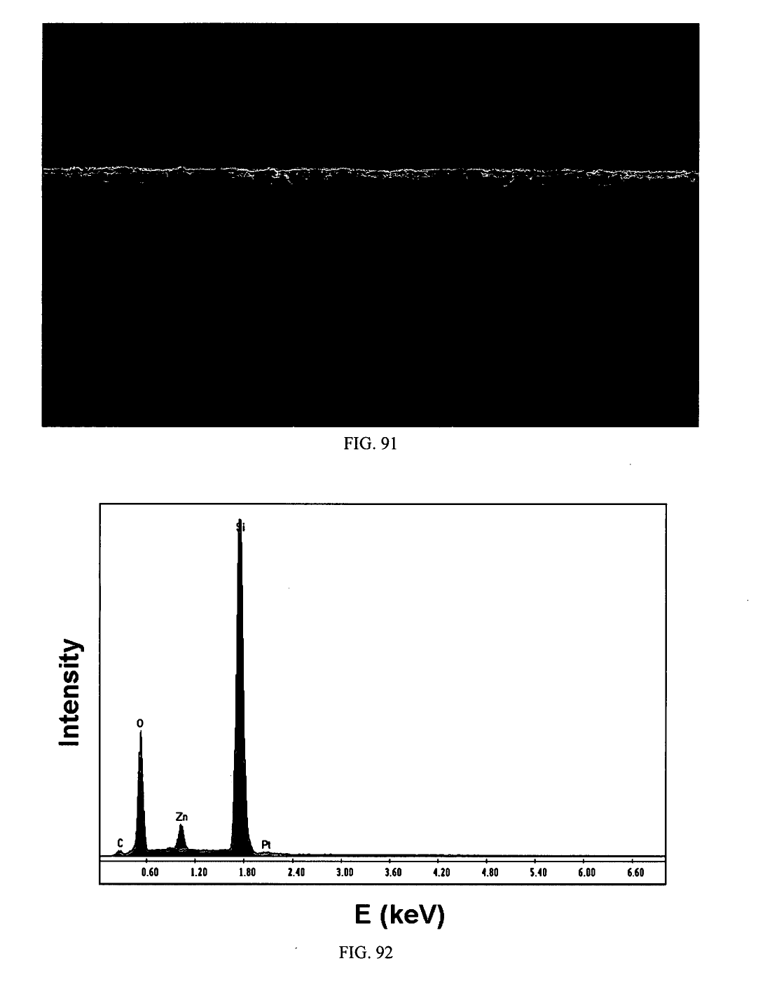

ZnO thin films were formed after exposing the substrate under the chemical solution for one minute. FIG. 89 is a plan view SEM image of the annealed ZnO thin film. The image shows a uniform film consists of nanosized particles with some uniform distributed nanopores. A uniform thickness around 24 nm from the cross-sectional image of ZnO thin film was shown in FIG. 90. The corresponding EDS spectrum is shown in FIG. 91. The film contains O and Zn, with some trace amounts of C. The background spectrum was taken at the margins of the sample where the substrate had no film deposition which contained Si, C, O, and Pt. Pt is an artifact of a conductive coating, applied to limit the effects of charging during analysis. The TEM and the SEM results suggest the films were formed through a nanoparticle sticking mechanism. The deposition of ZnO from aqueous solution (CBD) involves controlled precipitation on a substrate via hydrolysis and condensation reactions. The film morphology strongly depends on the experimental conditions such as ligand, pH, reactant concentrations, temperature, and the nature of the substrate. Like many other CBD processes, ZnO CBD is normally carried out as a batch process and involves both heterogeneous and homogeneous precipitation. Furthermore, the bath conditions change progressively as a function of time. Enclosed embodiments of the present invention provide a steady-state flux for the growth of nanostructured ZnO thin films from an aqueous solution in a fairly controlled manner.

The phase and crystalline orientation of the ZnO thin film deposited on an oxidized silicon substrate after annealing at 600° C. for 30 minutes was determined by the XRD measurement. FIG. 92 is an XRD spectrum ranging from 2θ=10° to 20° and shows an amorphous background from SiO₂. The peak at 20θ≈33° comes from the (331) peak of the single crystal silicon substrate (JCPDS-ICDD card No. 01-0791 and 01-0787). The peaks from the thin film at 2θ≈32°, 34°, 36°, and 47° agree with the (100), (002), (101), and (102) peaks of hexagonal ZnO structure, respectively (JCPDS-ICDD card No. 79-2205). The XRD spectrum indicates that the ZnO thin film structure is polycrystalline with a preferred (002) orientation.

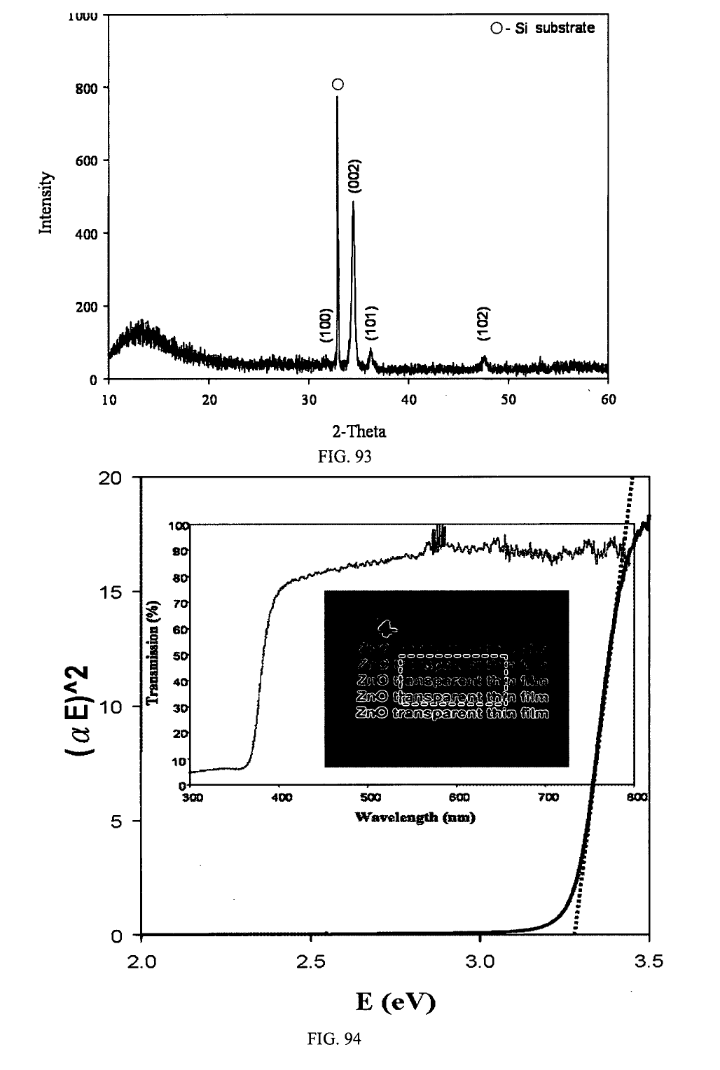

The optical bandgap of the ZnO thin film that was estimated from the UV-Vis absorption spectrum inset. FIG. 93 provides a plot of (α·hv)₂ versus hv from the ZnO thin film deposited on a glass slide after a thermal annealing in the air at 600° C. for 30 minutes. Extrapolation of the linear regime of the curve to (α·hv)²=0 gives an estimated optical bandgap value of 3.27 eV. This value is in agreement with the reported bandgap value of 3.35 eV for ZnO. Transmittance of the ZnO thin film (inset of FIG. 93) was measured in the wavelength range from 300 to 800 nm and shows a highly transparent ZnO thin film with an average value of 85% over 400 nm.

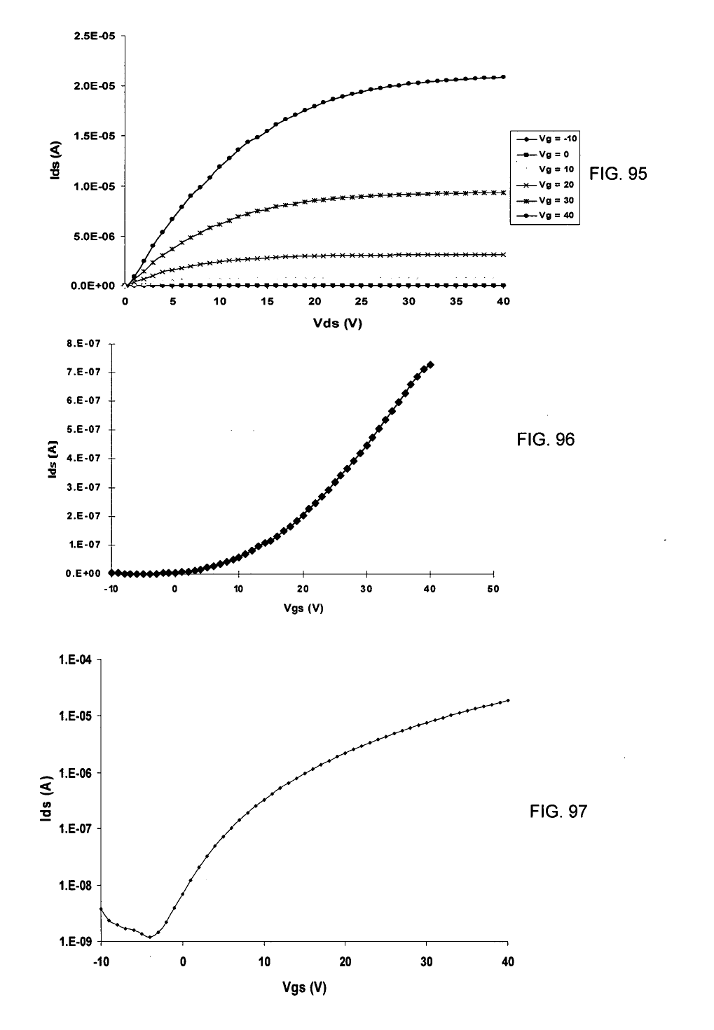

The MISFET was characterized and extracted important parameters including threshold voltage, mobility, drain current on-to-off ratio, and turn-on voltage. The drain current−drain voltage (I_{DS}−V_{DS}) output characteristics are presented in FIG. 94, which shows a good gate-modulated transistor behavior with a hard saturation. The threshold voltage of this device is approximated using a linear extrapolation method with the drain current measured as a function of gate voltage at a low V_{DS} to ensure an operation in the linear region. FIG. 95 shows the drain current−gate voltage (I_{DS}−V_{GS}) at V_{DS}=1 V using the linear extrapolation method for the threshold estimation, resulting in a threshold voltage of V_{T}=15 V.

The mobility of a MISFET refers to the carrier mobility that is proportional to the carrier velocity in an electric field. The effective mobility (μ_{eff}) is the most common mobility reported and depends on lattice scattering, ionized impurity scattering, and surface scattering and is derived from the drain conductance. The effective mobility for the ZnO device presented here has a value of μ_{eff}≅0.16 cm²/Vs.

The drain current on-to-off ratio determines the switching quality of the MISFET. FIG. 96 shows the Log(I_{DS})−V_{GS} transfer characteristics at V_{DS}=40 V indicating an drain current on-to-off ratio of approximately 10⁴ with a turn-on voltage at −4V. With a positive threshold voltage and negative turn on voltage, this device still behaves as a depletion-mode device that is initially on and requires a negative gate voltage to fully turn off the device. Recently, Sun and Sirringhaus reported solution-processed (spin-coating) ZnO field-effect transistors based on self-assembly of colloidal nanorods and nanospheres. An effective mobility≅0.23 cm²/Vs and a drain current on-to-off ratio of nearly 10⁶ were measured in air using the ZnO MISFET annealed in N₂/H₂ at 230° C. for 10 min and an additional hydrothermal anneal at 90° C. for 50 min. Better performance (μₛₐₜ=0.61 cm²/Vs) was observed when the devices were characterized in a nitrogen glovebox. This effect was attributed to the interaction between ZnO and CO₂. Lower mobilities (2.37×10⁻⁴˜4.62×10⁻⁴ cm²/Vs) were obtained from devices made from the nanospheres. Our ZnO device performance is comparable, however, further improvement of the device performance could be expected from an additional hydrothermal annealing. In addition, their results also suggest fabricating ZnO MISFETs using rod shape nanoparticles could be beneficial.

A newly developed chemical nanoparticle deposition process was used to deposit ZnO thin films at low temperature (˜80° C.). ZnO nanocrystals could be clearly observed from chemical solution from the microreactor according to the TEM images. The TEM results indicate the occurrence of homogeneous particle formation in the microchannel that is responsible for the thin film growth. The resulting film consists of highly transparent nanocrystalline ZnO with a hexagonal structure.

Functional ZnO MISFETs were fabricated using this experimental setup after a post annealing process at 600° C. for 30 minutes. An effective mobility, μ_{eff}≅0.16 cm²/Vs, a threshold voltage of 15 V, turn-on voltage of −4 V, and current on-to-off ratio of ˜10⁴ are obtained from the MISFET.

### EXAMPLE 3

This example describes one embodiment of a method for synthesizing 0.8 nm phosphine-stabilized Au nanoparticles (Au11) using a static micromixer with and without gas segments using an embodiment of an interdigital micromixer. NaBH₄ (2.02 mmol) and AuCl(PPh₃) (2.02 mmol) in absolute ethanol were fed into a static micromixer at a flow rate of (0.4 μL/min) with a resulting residence time of 45 seconds. The collected product from the micromixer was poured into hexanes and allowed to precipitate over night. The brown solid was collected and washed with hexanes, CH₂Cl₂/hexanes (1:1) and CH₂Cl₂/hexanes (3:1). The remaining solid was dissolved in CH₂Cl₂ and filtered a second time.

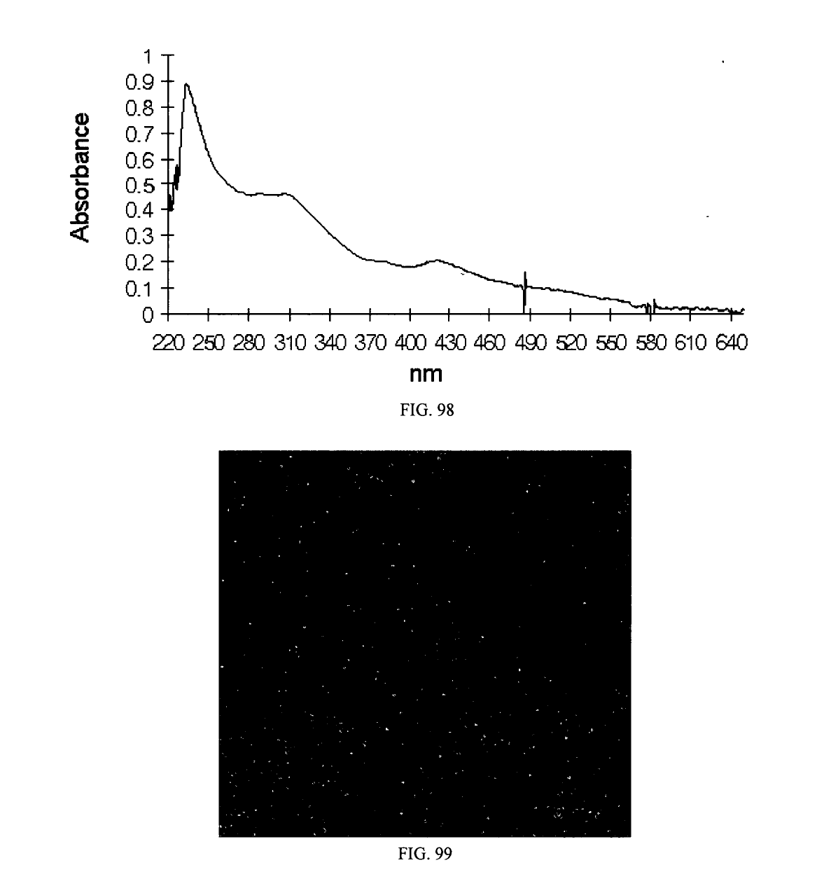

The solutions were characterized by UV-Vis absorption. The UV-Vis spectra of FIG. 97 shows the signature peaks of Au11 core. FIG. 98 is a transmission electron microscopy image used to provide direct images of nanoparticles. TEM image confirmed the formation of Au NPs with uniform size distribution. Segmented flow was implemented to provide better defined residence time distribution. The UV-vis spectra indicated that the segmented flow reactor produced Au nanoparticles with more pronounced signature peaks of Au11 core.

Organic solvent nanofiltration (OSNF) has been identified as a size-based separation method with potential application in the micromanufacture of nanoparticles. One such OSNF membrane, STARMEM 122 (Membrane Extraction Technology, London, UK), has been reported in recent literature to have excellent organic solvent chemical compatibility, good permeability of solvents such as methanol and toluene, and a molecular weight cutoff (MWCO) of 220 Da. These published studies have been conducted in the pressure range of 30-60 bar (435-870 psi). However, microfluidic devices typically operate in a range of lower pressures<5 bar (˜70 psi). STARMEM 122 membrane may be imbedded into a microfluidic device operated in a pressure range of <70 psi for size-based separations of nanoparticles. STARMEM 122 membrane can be laser welded directly to a polycarbonate substrate. This welding method eliminates the gaskets and mechanical clamps currently needed to maintain a seal under pressure. A linear trend in the permeation of methanol across the membrane has been found at pressures between 40-100 psi. A rejection of 94% was found for a surrogate molecule, Rhodamine B (MW 479 Da), at a pressure of 100 psi.

Fabrication efforts involve fabrication of the micromixer and microextraction modules necessary to react and separate Au nanoparticles as well as the interconnection necessary to implement a system of modules for continuous flow production of Au nanoparticles. Methods for fabricating hydrodynamic focusing and interdigital micromixers are being developed. A membrane-based chromatographic separator is being developed to separate products from the mixture. Physical extraction of the particles will be implemented using PDMS microvalves, that are fabricated by compressing micromolded PDMS laminae between micromilled polycarbonate via laser welding. Finite element analysis (FEA) models are being developed to assist in the design of these valves for various extraction and injection applications. LabVIEW is being used to establish a test bench for evaluating these valves.

### EXAMPLE 4

This example concerns synthesis of ceria (CeO₂) as an example of a method for making ceramic materials using disclosed nanofactory embodiments. One use for such materials is antireflective coatings. Fabrication techniques such as metal-organic chemical vapor deposition (Carter et al. 1999), electron beam evaporation (Inoue et al. 1992), sputter deposition (Jarrendahl et al. 1998), and pulsed laser ablation/deposition (Develos et al. 1998) generally produce poor coatings due their dependencies on process conditions. The refractive index of the conventionally evaporated films (Al-Robaee et al. 1992) was around 2.0, which is much less then the bulk index of 2.5. This has been attributed to voids created during columnar growth, which result in poor packing density (Kanakaraju et al. 1997). In most cases, these voids tend to absorb moisture that causes inhomogeneity of the film. In other words, the refractive index is changed when exposed to atmosphere. These problems limit the use of ceria in optical devices. In contrast, ion-assisted deposition (IAD) has been extensively studied to provide energetic bombardment of reactive/non-reactive ions during the film growth. This has been proved to effectively eliminating columnar microstructures. By supplying adequate energy to bombard the film, the columns in the film will collapse, which leads to densification of the films (Kanakaraju et al. 1997). However, this technique still has the limitation of coating a uniform surface over large areas.

Wet chemical deposition allows good control of the microstructure and uniformity over large coating areas. This is can be achieved by complexing the precursor, changing the pH and viscosity of the solution, and modifying the drying conditions (Ozer et al. 1995). It is expected that the particles synthesized in the micromixer will yield similar results as the wet chemical approach.

Film Formation by Wet Chemical Deposition

For the wet chemical approach, two kinds of methods are generally used to coat films: dip coating and spin coating. In dip coating, the substrate is immersed in the solution and is drawn up vertically. The solution dragged by the substrate is immediately dried and solidified into a gel film. In spin coating, an appropriate amount of solution is dropped on the rotating substrate and the solution distributes outward due to centrifugal forces (Sakka 1996). The bonding mechanism for the wet chemical approach is to form chemical bonds such as -M′-O-M-, where M′ and M are metallic ions in the film and in the substrate, respectively. There are several ways to activate the surface of the substrate, such as plasma oxidation, chemical treatment, and high temperature heating.

The disadvantage of this design is that only particular wavelength of light can be blocked out due to the design of the coating thickness. In addition, the strict tolerances have to be applied to each coating thickness during fabrication. Another problem for this kind of design is that theoretically it only works better in low incident angle (close to perpendicular) because the travel distance increases when the incident angle increases.

Sub-Wavelength Structure

The concept of the sub-wavelength structure is to produce a structure that gradually changes the index of refraction from one medium to another (e.g. air to glass). If the light goes from air (n₁=1) to glass (n₂=1.5), the reflectance can be calculated as 4%. On the other hand, if there is one coating with index of refraction of 1.25, the reflectance can be calculated as 1.235% (n₁=1 to n₂=1.25)+0.826% (n₁=1.25 to n₂=1.5). This gives us total reflectance of 2.06%, which is already 50% reduction with a single layer of coating. If the number of the transition layers were gradually increased, the total reflectance can be reduced.

To achieve gradual change of refraction index, the concept of sub-wavelength surface was introduced. This concept was initially observed in nature on the eyes of night-flying moths; accordingly, the structure is also called “moth-eye.” By periodically producing these sub-wavelength structures, the refractive index can be changed. The theory is that since the periodic structures were much smaller than the wavelength, the light treats the surface as a flat surface with weight-averaged index of refraction. For example if a first layer had only about 0.6% of oxide, the index of refraction can be calculated as
(0.6%×1.5)+1.00=1.01 (Layer 1: n=1.01)
According to this finding, the sub-wavelength structure must have gradient structures in order to achieve transition of refractive index. Nanorods had been successfully observed and coated onto the substrate using the micromixer, it is possible to deposit a sub-wavelength and maybe gradual change of refractive index.

In this example, a traditional batch precipitation approach for ceria nanoparticle synthesis (Zhou et al., 2002) was compared to ceramic nanoparticle synthesized using a microchannel nanofactory comprising a T-mixer. By varying reactant concentrations, different nanoparticle sizes and morphologies were obtained using nanofactories as compared to conventional batch mixer processes.

Precipitation synthesis generally involves formation of an intermediate. For example, Ce(NO₃)₃ can be used as a starting material for ceria production. Intermediates, such as Ce(OH)₃ or Ce(OH)₄ can form when using this starting material. In most cases, these precursor powders must be thermally decomposed to obtain the desired ceramic powder. These intermediate precursor powders have to be separated, such as by drying and calcination.

Both a batch mixer and a microchannel T-mixer were used and reactant concentrations were varied for each mixer configuration. Two sets of concentration levels were used. The batch mixer and T-mixer methods was used in experiment (i) and (ii). In experiment (i), a greater higher amount (0.0375M) of cerium nitrate was used and 3 mL of NH₄OH were added. For experiment (ii), a smaller amount (0.0187M) of cerium nitrate was used and 5 mL of NH₄OH were added.

An analytical balance (Acculab AL Analytical Series AL-104) was used to measure the amount of cerium(III) nitrate. The quantity of ammonium hydroxide was measured then added to the batch mixer using a pipette (EPPENDORF® REPEATER™ PLUS Pipettor).

A peristaltic pump (ISM 833, Upchurch) was used to pump the reactants through the T-mixer. By using a peristaltic pump, the reactants were not exposed to the pumping mechanism, which prevents clogging by reducing contamination. r

For the batch mixer, cerium(III) nitrate was dissolved in deionized water then ammonium hydroxide (NH₄OH) was added by pipette until the pH was above 10. When the pH>10, precipitation/nucleation of ceria nanoparticles occurred, followed by particle growth. The difference between the two experiments (i and ii) for the batch mixer is the molar concentration level. By changing the [Ce³⁺] and [OH⁻] concentration, the supersaturation value S is changed. When [OH⁻] increases, the supersaturation value increases significantly. In theory, higher supersaturation values result in smaller nanoparticle.

For the batch mixer, 99.9% Ce(NO₃)₃ was used. For the first trial, 0.4066 gram was used, and in the second trial, 0.2035 gram was used. For the first trial, 12 250 microliter aliquots were added, for a total addition of 3 milliliters. For the second trial, 20 aliquots of 250 microliters were added, for a total of 5 milliliters. These reaction compositions were then diluted with deionized water to provide 24 total milliliters. The first reaction mixture was stirred using a magnetic stirrer for 150 minutes. The second reaction mixture was stirred for 37 minutes. The molar mass for cerium nitrate is 434.22 g/mole, and for the first trial 0.0375M of cerium nitrate was used then 3 mL of NH₄OH was added. For experiment (ii), 0.0187M of cerium nitrate was used then 5 mL of NH₄OH was applied.

The peristaltic pump can only function when the two reactants have equal volume amounts at the same flow rate. As a result, different amounts of deionized water were used to dissolve cerium(III) nitrate and to dilute ammonium hydroxide. The amounts of reactants and solvents used in the T-mixer were about the same as for the batch mixer trials. For the first trial, 0.4090 gram of Ce(N)₃)₃ was used, and in the second trial, 0.2035 gram was used. For the first trial, 3 milliliters of 5 N NH4OH was used, an in the second trial, 3 milliliters were used. 12.5 milliliters of deionized water was used in the first, and the peristaltic pump operated at 7 milliliters/minute. For the second trial, 20 aliquots of 250 microliters were added, for a total of 5 milliliters. These reaction compositions were then diluted with deionized water to provide 24 total milliliters. The first reaction mixture was stirred using a magnetic stirrer for 150 minutes. The second reaction mixture was stirred for 37 minutes. The molar mass for cerium nitrate is 434.22 g/mole, and for the first trial 0.0375M of cerium nitrate was used then 3 Ml of NH₄OH was added. For experiment (ii), 0.0187M of cerium nitrate was used then 5 Ml of NH₄OH was applied.

Again, two levels of conditions were performed in the T-mixer approach. For experiment (i), given that the molar mass for cerium nitrate is 434.22 g/mole, 0.0375M of cerium nitrate was used then 3 mL of NH₄OH was applied. For experiment (ii), 0.0187M of cerium nitrate was used then 5 mL of NH₄OH was applied.

The morphology of prepared CeO₂ nanoparticles was analyzed using transmission electron microscope (TEM, FEI Tecnai F-20 field emission high resolution) at Portland State University. The sample was prepared by applying an appropriate amount of CeO₂ suspension on the copper grid with tissue paper on the bottom. The particle size and particle size distribution are measured manually or by software.

After the nanoparticles were deposited onto the substrate, a Zeiss Ultra scanning electron microscope (SEM, Micro Analytical Facility, CAMCOR, Univ. of Oregon) was used to examine the structure of the cerium oxide. Furthermore, the elemental distribution of the sample surface also was determined by energy dispersive X-ray spectroscopy (EDS).

To characterize the crystalline structure of the dried particles, X-ray diffractometer (XRD) will be used. The crystallite size d_{XRD} of the samples also can be estimated from XRD patterns by applying full-width-half-maximum (FWHM) of characteristic peak (1 1 1) using the Scherrer equation. The chemical (oxidation) state of nanoparticles can be determined by X-ray photoelectron spectroscopy (XPS).

The batch mixer provided relatively poor mixing, and hence longer reaction times were needed. It required about 2.5 hours to form light yellow precipitates (CeO₂) in experiment (i) compared to 37 minutes in experiment (ii) having higher concentration of [OH⁻].

With a flow rate of 7 mL/min, the T-mixer finished the process within 2.5 minutes in both trials. Both resultant nanoparticles were purple colored instead of yellow as with the batch mixing experiment. Based on past work, this suggests the presence of Ce(OH)₃ (Chen 2004). Further characterization needs to be performed by XPS to verify the composition of the purple nanoparticles. This is an interesting result suggesting an additional potential benefit to the microreactor approach. In the batch reaction, the time needed to mix the reactants causes enough exposure to ambient oxygen that the process progresses directly to its end product (CeO₂). However, within the micromixer, Ce(OH)₃ precipitates, and the purple nanoparticles remain as Ce(OH)₃ configuration as long as they are stored without exposure to air. After depositing these materials on a surface and drying, the Ce(OH)₃ transitions to CeO₂, yielding a resultant color change to light yellow.

Scanning Electron Microscopy (SEM)

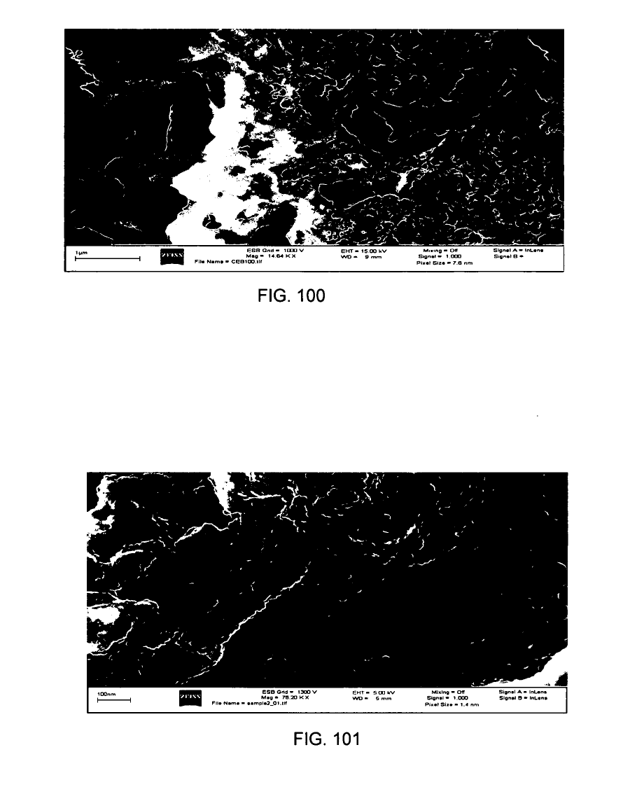

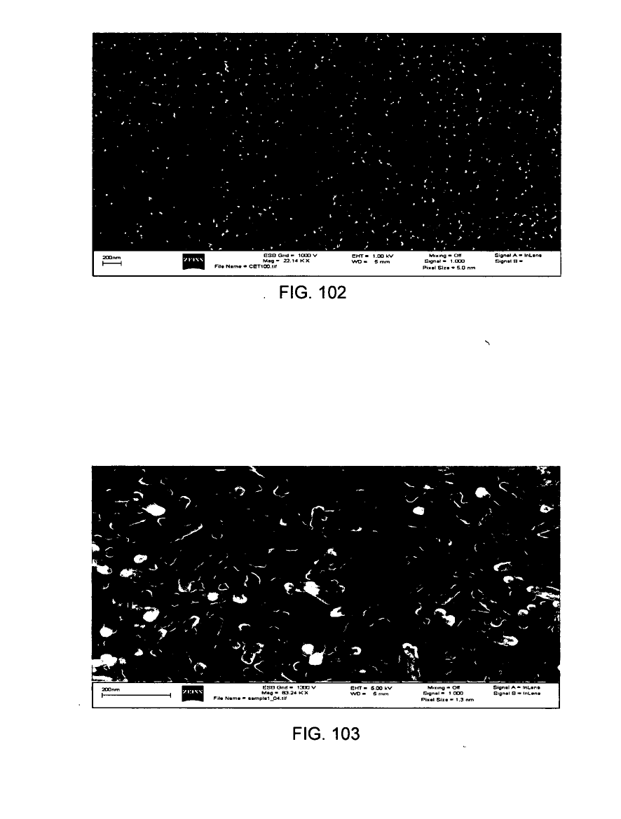

The SEM images (FIGS. 99-102) show significant differences between nanoparticle morphologies. The batch mixer tends to have smaller particles but the particle size distribution tends to be bigger and the structure is more complex. The T-mixer structure appears to be “sticks” instead of round features when the [Ce³⁺ ] molar ratio is lower. The supersaturation value may have to be within a certain value to precipitate round nanoparticles. One explanation for the “stick” shaped ceria is due to the high supersaturation value which caused fast particle growth along the preferred crystalline structure. In addition from the morphologies, the nanoparticles from the batch mixer also show more agglomeration when compared to particles made using disclosed embodiments of the nanofactory.

Energy Dispersive X-ray Spectroscopy (EDS)

Though the structures and the morphologies of the nanoparticles were diversely different between the batch and T-mixer, XPS and EDS results show high percentage of Ce and O indicating the existence of CeO₂.

Transmission Electron Microscope (TEM)

The TEM results show that the batch mixer produced an average particle size of about 5 nm. Nanoparticles made according to the present disclosed embodiments had an average particle size of about 8 nm. However, according to HRTEM images, nanoparticles made according to the present disclosed embodiments had better crystallinity compared to the batch mixer. In addition, since the samples prepared for TEM study were not calcined, the nanorod structure was formed before the deposition process.

The present invention has been described with reference to certain exemplary embodiments. The present invention should not be limited to these disclosed embodiments, but rather should be accorded the scope understood by a person of ordinary skill in the art in view of the disclosure and the following claims.

We claim:

1. An apparatus for chemical synthesis and/or analysis, comprising a laminated, microfluidic structure defining a reactor and a separator.

2. The apparatus according to claim 1 where the reactor comprises a mixer and temperature control section.

3. The apparatus according to claim 1 further comprising at least two unit operations selected from a mixer, a heating system, a cooling system, a valve, and a detector.

4. The apparatus according to claim 3 where the mixer includes plural channels having a width of about 50 μm or less and a length of about 250 μm or less.

5. The device according to claim 3 comprising a fluidly actuatable valve having a fluidly deflectable elastomeric layer.

6. The apparatus according to claim 1 having plural, selectively actuatable valves.

7. The apparatus according to claim 1 wherein the separator is selected from a dielectrophoretic separator, an electrophoretic separator, a templated, sorbent-based separator, a non-templated separator, a capillary electrochromatographic separator, a capillary zone electrophoretic separator, a dendrimer templated separator and combinations thereof.

8. The microchemical nanofactory according to claim 1, comprising:
at least a first fluid inlet and a second fluid inlet for feeding a first reagent and a second reagent to a mixer, which forms a mixture;
a heating or cooling zone for receiving the mixture; and
a separator for separating a product formed by reaction of the first reagent and second reagent from other materials.

9. The microchemical nanofactory according to claim 8 where the mixer is an interdigital mixer and the mixture flows perpendicularly to directions defined by flow of the first reagent and the second reagent from a first layer comprising the mixer to a second layer positioned adjacent the first layer.

10. The microchemical nanofactory according to claim 1, comprising:
at least a first inlet and a second inlet for feeding a first reagent and a second regent to a mixer, which forms a mixture;
an optional heating or cooling zone for receiving the mixture;
a first microchannel for receiving a product and other materials, the first microchannel being fluidly coupled to a first separator;
a second microchannel for receiving the product and the other materials, the second microchannel being fluidly coupled to a second separator;
selectively actuatable valves operatively coupled to the first microchannel and the second microchannel for guiding the product and the other materials to the first separator and to the second separator for separating product from the other materials to form a separated product and separated materials;
plural microchannels operatively coupled to the first and second separators to receive the product and the separated materials; and
plural, selectively actuatable valves operatively coupled to the plural microchannels for guiding the product to a product microchannel and the separated materials to a separated materials microchannel.

11. The microchemical nanofactory according to claim 1, comprising:
at least a first fluid inlet and a second fluid inlet for feeding a first fluid and a second fluid to a mixer;
a microchannel for feeding a mixed fluid stream from the mixer to an optional heater or cooler;
a first microchannel for feeding a first fluid product stream comprising the product and other materials to a first separator;
a second microchannel for feeding a second fluid product stream comprising the product and other materials to a second separator;
fluidly actuatable valves selectively actuatable for flowing the first fluid product stream to the first separator and the second fluid product stream to the second separator, where product is separated from other material or materials in the first separator and/or the second separator to form a separated product fluid stream and a separated material stream;
plural microchannels fluid coupled to the first and second separators to receive a separated product fluid stream and a separated material fluid stream comprising material separated from the product; and
plural, fluidly actuatable valves selectively actuatable for guiding product flow into a product microchannel and separated materials flow to a separated materials microchannel.

12. A system for making chemical compounds comprising plural microchemical nanofactories coupled in parallel or in series, at least one nanofactory comprising a first laminated, microfluidic structure defining a reactor and a separator.

13. A method for making chemical compounds, comprising:
providing a laminated, microfluidic apparatus defining a reactor and a separator;
providing reagents to the apparatus appropriate for making the chemical compound; and
operating the apparatus to make the desired compound.

14. The method according to claim 13 where the chemical compound is a dendrimer, and the reagents comprise ethylene diamine and methylacrylate acid.

15. The method according to claim 13 where the compound made is selected from the group consisting of oligomers, biological macromolecules, simple and complex natural products, supermolecules, commercial polymeric materials, respiratory stimulants, analgesics, behavior-modifying agents, anesthetic agents, anticonvulsants, muscle relaxants, antiarrhythmic drugs, ACE inhibitors, calcium channel blocking agents, vasodilating agents, alpha-adrenergic blocking agents, beta-adrenergic blocking agents, angiotensin converting enzyme blockers, antihypertensive agents, sympathomimetics, bronchodilators, xanthines, antihistamines, antitussives, mucolytics, diuretics; carbonic anhydrase inhibitors, urinary alkalinizers, urinary acidifiers, cholinergic stimulants, urolithiasis agents, antiemetic agents, antacids, H2 antagonists, gastromucosal protectants, prostaglandin E1 analogs, proton pump inhibitors, G1 antispasmodics/anticholinergics, G1 stimulants, digestive enzymes, antidiarrheals, sex hormones, posterior pituitary hormones, oxytocics, adrenal cortical steroids, mineralocorticoids, glucocorticoids, adrenal steroid inhibitors, anti-diabetic agents, thyroid hormones, endocrine/reproductive drugs, prostaglandins, antiparasitics, anticoccidial agents, aminocyclitols, macrolides, penicillins, tetracyclines, lincosamides, quinolones, sulfonamides, antibacterials, antifungal agents, antiviral agents, clotting agents, anticoagulants, erythropoietic agents, blood modifying agents, alkylating agents, antimetabolites; mitotic inhibitors, antineoplastics, and immunosuppresive drugs.

16. The method according to claim 13 further comprising providing plural systems coupled in parallel or in series.

17. The method according to claim 13, comprising:
providing a first microchemical nanofactory;
using the nanofactory to produce a chemical compound or an intermediate useful for making a desired chemical compound; and
performing at least one operation useful for making the desired chemical compound without using the nanofactory.

18. The method according to claim 17 comprising performing a separation or purification process using an apparatus other than the microchemical nanofactory.

19. The method according to claim 13, comprising:
providing a first microchemical nanofactory;
using the nanofactory to produce a desired chemical compound;
using the chemical compound in a downstream process.

20. The method according to claim 13 useful for making a nanostructured photovoltaic, comprising:
providing a substrate; and
depositing a p-QD adsorber layer on to the substrate.
